(19) United States
(12) Patent Application Publication
(10) Pub. No.: US 2003/0157558 A1
(43) Pub. Date: Aug. 21, 2003

(54) Novel guanosine triphosphate-binding protein-coupled receptors, genes thereof and production and use of the same

(76) Inventors: Shun-ichiro Matsumoto, Chiba (JP); Tamaki Oda, Ibaraki (JP); Youko Saito, Ibaraki (JP); Noriyuki Morikawa, Ibaraki (JP); Kenji Yoshida, Ibaraki (JP); Makiko Suwa, Tokyo (JP); Tomoyasu Sugiyama, Tokyo (JP); Toshimitsu Kishimoto, Tokyo (JP); Kouji Kanzaki, Tokyo (JP); Shin-ichiro Yasuda, Tokyo (JP); Yoshihisa Inoue, Tokyo (JP)

Correspondence Address: KLARQUIST SPARKMAN, LLP, 121 SW SALMON STREET, SUITE 1600, PORTLAND, OR, 97204, US

(21) Appl. No.: 10/088,726

(22) PCT Filed: Dec. 28, 2000
(86) PCT No.: PCT/JP00/09408

(30) Foreign Application Priority Data
Dec. 28, 1999 (JP) 11/375152
Mar. 31, 2000 (JP) 2000/101337

(51) Int. Cl. G01N 33/53; C07H 21/04; C07K 14/705; C12P 21/02; C12N 5/06

(52) U.S. Cl. 435/7.1; 435/69.1; 435/320.1; 435/325; 530/350; 536/23.5

(57) ABSTRACT

Nine novel genes sustaining hydrophobic domains, which are estimated to be seven transmembrane domains characteristic to G protein-coupled receptors, are successfully isolated by human tissue cDNA screening. These genes and proteins which are the expression products thereof are usable in screening ligands, screening agonists or antagonists which are useful as drugs, diagnosing diseases in which these gene participate, etc.

### TECHNICAL FIELD

The present invention relates to novel G protein-coupled receptors and genes thereof, and production and uses thereof.

### BACKGROUND ART

G protein-coupled receptor is a generic name for the group of cell membrane receptors transducing signals into cells via the activation of trimer-type GTP-binding proteins. The G protein-coupled receptor has structural characteristic of seven transmembrane domains in a molecule, and thus called also as “a seven-transmembrane receptor”. The G protein-coupled receptor transmits the information consisting of various physiologically active substances into cells across the cell membrane via the activation of the trimer-type GTP-binding protein and the change of the intracellular second messengers caused thereby. Well-known intracellular second messengers that are regulated by the trimer-type GTP-binding protein include cAMP mediated by adenylate cyclase, and Ca²⁺ mediated by phospholipase C. Recent studies have shown that many types of intracellular proteins serve as the targets thereof; for example, the regulation of channels and activation of phosphorylation enzymes are mediated by the trimer-type GTP-binding protein (Annu. Rev. Neurosci. (97) 20:399). There are a wide variety of substrates (ligands) for the G protein-coupled receptor, for example, protein hormone; chemokine; peptide; amine; substances derived from lipids; and protease, such as thrombin, is also one such example. The number of human G protein-coupled receptors whose genes have been identified recently, is a little under 300, excluding the sensory-type receptors. However, the number of G protein-coupled receptors to which the ligands have been identified is only about 140 types. Thus, there are 100 or more, ligand-unknown, “orphan G protein-coupled receptors”. The human genome has been assumed to contain at least 400 types, and possibly up to 1000 types of G protein-coupled receptors (Trends Pharmacol. Sci. (97) 18:430) This means that the number of functionally unknown orphan G protein-coupled receptors can be exploding accompanied by the rapid progress of the genome analysis.

Ninety % or more drugs that have so far been produced by the pharmaceutical companies in the world aim at the interaction in extracellular spaces, and low-molecular-weight drugs comprises the majority of those relating to G protein-coupled receptors. The reason is that the G protein-coupled receptor-related diseases include many types of diseases, such as those of the cerebral nervous system, circulatory system, digestive system, immune system, locomotor system, urinary system, and genital system, including genetic diseases. Thus, in recent years, many pharmaceutical companies retain their orphan G protein-coupled receptors found through the genome analysis, and are competing fiercely with each other to reveal the ligands and physiological functions. Based on this, successful cases of physiological screening of ligands to some novel G protein-coupled receptors have begun to be reported recently. For example, the cases of a calcitonin-related peptide receptor (J. Biol. Chem. (96) 271:11325) , orexin (Cell (98) 92:573) , and prolactin-releasing peptide (Nature (98) 393:272) gave a great impact to basic studies in the field of life science.

In particular, as potential new targets to bring about the drug development, the orphan G protein-coupled receptors have become a center of attraction. In general, since there are no specific ligands to the orphan G protein-coupled receptors, it has been difficult to develop agonists or antagonists. However, in recent years, creation of orphan G protein-coupled receptor-targeted drugs by combining the enriched compound libraries and high-throughput screening methods has been proposed (Trends Pharmacol. Sci. (97) 18:430, Br. J. Pharm. (98) 125:1387). Specifically, in the creation comprises identifying physiological agonists of an orphan G protein-coupled receptor identified by genetic engineering, by functional screening utilizing alterations in the level of an intracellularmessenger, cAMP or Ca²⁺, as an index, and then analyzing the in vivo functions. In this method, high-throughput screening achieved by using a compound library allows theoretically to discover surrogate agonists and antagonists specific to the orphan G protein-coupled receptor, and further, to develop therapeutic agents for particular diseases.

### DISCLOSURE OF THE INVENTION

The present invention was achieved considering the present situation surrounding G protein-coupled receptors, and an objective thereof is to provide novel G protein-coupled receptors and their genes, and a method for producing and uses of them. Another objective of the present invention is to provide these molecules as targets for the study of drug development.

The present inventors studied strenuously to achieve the above-mentioned objectives, and successfully isolated nine novel genes comprising nucleotide sequences encoding hydrophobic regions considered to be seven transmembrane domains, which are characteristic of the G protein-coupled receptors, by polymerase chain reaction using cDNAs from human tissues as templates. These genes and the proteins as the translation products can be used in the screening of ligand and of agonist or antagonist useful as a pharmaceutical, or can be used for diagnosing diseases relating to these genes.

Thus, the present invention relates to novel G protein-coupled receptors and the genes encoding them, and the uses and production thereof. More specifically, the present invention provides:

(1) a DNA that encodes a guanosine triphosphate-binding protein-coupled receptor, wherein said DNA is selected from the group consisting of the following (a) to (d):

(a) a DNA encoding a protein comprising the amino acid sequence of any one of SEQ ID NOs: 1 to 4 and 17 to 21;

(b) a DNA comprising a coding region of the nucleotide sequence of any one of SEQ ID NOs: 5 to 8 and 22 to 26;

(c) a DNA encoding a protein comprising the amino acid sequence of any one of SEQ ID NOs: 1 to 4 and 17 to 21 in which one or more amino acids are substituted, deleted, added, and/or inserted; and

(d) a DNA hybridizing under stringent conditions to the DNA comprising the nucleotide sequence of any one of SEQ ID NOs: 5 to 8 and 22 to 26;

(2) a DNA encoding a partial peptide of a protein comprising the amino acid sequence of any one of SEQ ID NOs: 1 to 4 and 17 to 21;

(3) a vector comprising the DNA of any one of (1) and (2);

(4) a transformant carrying the DNA of any one of (1) and (2) or the vector of (3);

(5) a protein or a peptide encoded by the DNA of any one of (1) and (2);

(6) a method for producing the protein or the peptide of (5), said method comprising the steps of culturing the transformant of (4) and recovering an expressed protein or peptide from the transformant or culture supernatant thereof;

(7) a method of screening for ligands that bind to the protein of (5), said method comprising the steps of:

(a) contacting a test sample with the protein or the peptide of (5); and

(b) selecting compounds that binds to said protein or said peptide;

(8) a method of screening for compounds that have activity of inhibiting the binding between the protein of (5) and a ligand thereof, said method comprising the steps of:

(a) contacting the protein of (5) or a partial peptide thereof with the ligand in the presence of a test sample and detecting a binding activity of said protein or said partial peptide with said ligand; and

(b) selecting compounds that reduces the binding activity detected in step (a) as compared with a binding activity detected in the absence of the test sample;

(9) a method of screening for compounds that inhibit or enhance activity of the protein of (5), said method comprising the steps of:

(a) contacting a ligand of said protein with cells expressing said protein in the presence of a test sample,

(b) detecting an alteration in the cells that results from binding of said ligand to said protein, and

(c) selecting compounds that suppress or enhance the alteration detected in step (b) as compared with an alteration detected in the cells in the absence of the test sample;

(10) the method of (8) or (9), wherein the alteration in cells is a change in cAMP concentration or calcium concentration;

(11) an antibody binding to the protein of (5);

(12) a compound isolated by the method of any one of (7) to (10);

(13) a pharmaceutical composition comprising the compound of (12) as an active ingredient;

(14) the pharmaceutical composition of (13), wherein said pharmaceutical composition is formulated for the treatment of a disease selected from the group consisting of cancer, cirrhosis, and Alzheimer's disease;

(15) a polynucleotide comprising at least 15 nucleotides, wherein said polynucleotide is complementary to the DNA comprising the nucleotide sequence of any one of SEQ ID NOs: 5 to 8 and 22 to 26 or a complementary strand thereof;

(16) a method for diagnosing a disease selected from the group consisting of cancer, cirrhosis, and Alzheimer's disease, said method comprising the steps of detecting expression of the DNA of (1) in tissues related to the disease derived from a subject, or mutation in the DNA of (1) in the subject; and

(17) a agent for diagnosing a disease selected from the group consisting of cancer, cirrhosis, and Alzheimer's disease, said agent comprising the antibody of (11) or the nucleotide of (15).

As used herein, the term “G protein-coupled receptor” means a cell membrane receptor transducing signals into cells via the activation of the GTP-binding protein.

As used herein, the term “ligand” means a physiological substance binding to the G protein-coupled receptor and transducing signals into cells. Herein, the term “physiological substance” means a compound bound to the G protein-coupled receptor in vivo.

As used herein, the term “agonist” means a compound capable of binding to the G protein-coupled receptor and transducing signals into cells, including biological substances, artificially synthesized compounds, and naturally occurring compounds.

As used herein, the term “antagonist” means a compound inhibiting the binding of ligand to the G protein-coupled receptor or inhibiting the signal transduction into cells, including biological substances, artificially synthesized compounds, and naturally occurring compounds.

The present invention provides novel G protein-coupled receptors and the DNAs encoding the proteins. The nine human cDNA clones, isolated by the present inventors and included by the present invention, were named “GPRv8”, “GPRv12”, “GPRv16”, “GPRv21”, “GPRv40”, “GPRv47”, “GPRv51”, “GPRv71”, and “GPRv72”(as required, these clones are collectively referred to as “GPRv”) . The nucleotide sequences of the cDNAs are shown in SEQ ID NOs: 5 to 8 and 22 to 26; the amino acid sequences of the proteins encoded by the cDNAs are shown in SEQ ID NOs: 1 to 4 and 17 to 21.

A result obtained by BLAST search showed that amino acid sequence of all the proteins encoded by GPRv cDNAs exhibited significant homology to those of known G protein-coupled receptors. Specifically, “GPRv8” exhibited 36% homology to HUMAN VASOPRESSIN V1B RECEPTOR (P47901, 424 aa) ; “GPRv12” exhibited 27% homology to RAT 5-HYDROXYTRYPTAMINE 6 RECEPTOR (P31388, 436 aa); “GPRv16” exhibited 28% homology to MOUSE GALANIN RECEPTOR TYPE 1 (P56479, 348 aa); “GPRv21” exhibited 30% homology to BOVIN NEUROPEPTIDE Y RECEPTOR TYPE 2 (P79113, 384 aa) ; “GPRv40” exhibited 34% homology to OXYTOCIN RECEPTOR (P97926, 388 aa) ; “GPRv47” exhibited 43% homology to GPRX_ORYLA PROBABLE G PROTEIN-COUPLED RECEPTOR (Q91178, 428 aa); “GPRv51” exhibited 37% homology to PROBABLE G PROTEIN-COUPLED RECEPTOR RTA (P23749, 343 aa); “GPRv71” exhibited 45% homology to Chicken P2Y PURINOCEPTOR 3 (P2Y3) (Q98907, 328 aa); “GPRv72” exhibited 30% homology to ALPHA-1A ADRENERGIC RECEPTOR (O02824, 466 aa).

Further, all the proteins encoded by GPRv cDNAs (hereinafter also may be referred to as “GPRv protein”), isolated by the present inventors, contained hydrophobic regions, which were assumed to correspond to the seven transmembrane domains characteristic of the G protein-coupled receptor. Based on these findings, all the GPRv cDNAs can be considered to encode proteins belonging to the G protein-coupled receptor family. The G protein-coupled receptors have the activity for transducing signals into cells via the activation of the G protein, which is mediated by the ligand. As described above, the receptor are involved in many types of diseases, such as those of the cerebral nervous system, circulatory system, digestive system, immune system, locomotor system, urinary system, and genital system, including genetic diseases. Accordingly, the GPRv proteins can be used to screen for agonists and antagonists regulating the functions of GPRv proteins, and thus become important targets of drug development for the above diseases.

The present invention also provides proteins functionally equivalent to the GPRv proteins. As used herein, the term “functionally equivalent” means that a protein of interest has biological properties identical to those of the GPRv proteins. The biological properties of GPRv proteins include the activity of transducing signals into cells via the activation of the trimer-type GTP-binding protein. According to the types of activated systems of intracellular signal transduction, the trimer-type GTP-binding proteins are categorized into three classes, namely, Gq type that increases the Ca²⁺ level, Gs type that increases the cAMP level, and Gi type that reduces the cAMP level (Trends Pharmacol. Sci. (99) 20:118). Thus, it can be assessed whether the protein of interest has biological properties identical to those of GPRv proteins, for example, by detecting concentration changes of cAMP or calcium in cells depending on the activation.

In an embodiment, the method for preparing a protein functionally identical to the GPRv protein includes a method of introducing mutations in the amino acids sequence of the protein. Such method includes, for example, site-directed mutagenesis (Current Protocols in Molecular Biology, edit. Ausubel et al. (1987) Publish. John Wiley & Sons Section 8.1-8.5)). The amino acid mutations in the protein can be also occurred naturally. The present invention includes mutant proteins, regardless of being generated artificially or naturally, in which one or more amino acids have been substituted, deleted, inserted and/or added in the amino acid sequences (SEQ ID NOs: 1 to 4 and 17 to 21) of GPRv proteins, but the mutant proteins are functionally equivalent to the GPRv proteins. There is no limitation on the number of amino acid mutations and positions of the mutations in the proteins, as far as the functions of GPRv proteins are retained. The number of mutations is assumed to range typically within 10% of the entire amino acids, preferably within 5% of the entire amino acids, further preferably within 1% of the entire amino acids.

In another embodiment of the invention, the method for preparing a protein functionally equivalent to the GPRv protein includes a method using the hybridization technique or gene amplification technique. Specifically, those skilled in the art can typically isolate a DNA having high homology to the DNA sequence encoding the GPRv protein (SEQ ID NOs: 5 to 8 and 22 to 26) or a partial sequence thereof from a DNA sample derived from a homologous or heterologous using the hybridization technique (Current Protocols in Molecular Biology, edit. Ausubel et al. (1987) Publish. John Wiley & Sons Section 6.3-6.4) and then obtain a protein functionally equivalent to the GPRv protein. Thus, the protein of the present invention also include a protein encoded by a DNA capable of hybridizing to the DNA encoding the GPRv protein, which is functionally equivalent to the GPRv protein.

Organisms to be used for isolating such a protein include, for example, rat, mouse, rabbit, chicken, pig, cattle, and so forth, in addition to human, but not limited thereto.

Typical stringent hybridization conditions for isolating a DNA encoding a protein functionally equivalent to the GPRv protein are those of “1×SSC, 0.1% SDS, 37° C.” or the like; more stringently, those of “0.5×SSC, 0.1% SDS, 42° C.” or the like; much more stringently, those of “0.2×SSC, 0.1% SDS, 65° C.” or the like. As the hybridization conditions become more stringent, a DNA with higher homology to the probe sequence can be expected to be isolated. However, the above combinations of SSC, SDS, and temperature are only examples, and those skilled in the art can achieve the stringencies equivalent to the above by appropriately combining the above or other factors determining the hybridization stringency (for example, probe concentration, probe length, time of hybridization reaction, and so forth).

The protein encoded by a DNA isolated by using such hybridization technique typically has high homology of amino acid sequence to those of the GPRv protein. The term “high homology” means the degree of sequence homology of at least 40% or higher, preferably 60% or higher, further preferably 80% or higher (for example, 90% or higher, or 95% or higher).

Identity of amino acid sequence or nucleotide sequence can be determined with the BLAST algorithm of Karlin and Altschul (Proc. Natl. Acad. Sci. USA 90:5873-5877, 1993). Based on this algorithm, the programs, BLASTN and BLASTX, have been developed (Altschul et al. J. Mol. Biol. 215: 403-410, 1990). When nucleotide sequences are analyzed by BLASTN based on BLAST, the parameters are set, for example, as follows: score=100; and wordlength=12. Alternatively, when amino acid sequences are analyzed by BLASTX based on BLAST, the parameters are set, for example, as follows: score=50; and wordlength=3. When BLAST and the Gapped BLAST program are used for the analysis, the default parameters are used in each program. The specific techniques used in these analysis methods are already known (http://www.ncbi.nlm.nih.gov.).

Further, primers are designed based on a part of the DNA sequence (SEQ ID NOs: 5 to 8 and 22 to 26) encoding the GPRv protein, a DNA fragment having high homology to the DNA sequence encoding the GPRv protein is isolated by the gene amplification technique (PCR) (Current protocols in Molecular Biology, edit. Ausubel et al. (1987) Publish. John Wiley & Sons Section 6.1-6.4), and then the protein functionally equivalent to the GPRv protein can be obtained.

The present invention also includes partial peptides of the protein of the present invention. These partial peptides include peptides binding to the ligand but not transducing signals. An affinity column prepared using such a peptide can be used suitably for ligand screening. In addition, the partial peptides of the protein of the present invention can be used for preparing antibodies. The partial peptides of the present invention can be produced, for example, by using genetic engineering techniques, known peptide synthetic methods, or methods of digesting the protein of the present invention with an appropriate peptidase. The partial peptides of the present invention typically consist of 8 or more amino acid residues, preferably 12 or more amino acid residues (for example, 15 or more amino acid residues).

The protein of the present invention can be prepared as a recombinant protein or natural protein. The recombinant protein can be prepared, for example, as follows, by introducing a DNA encoding the protein of the present invention, which has been inserted in a vector, into an appropriate host cell and purifying the protein expressed in the transformant. On the other hand, the natural protein can be prepared, for example, by using the affinity column, in which an antibody against the protein of the present invention has been immobilized, as follows (Current Protocols in Molecular Biology, edit. Ausubel et al. (1987) Publish. John Wiley & Sons Section 16.1-16.19). The antibody to be used in the affinity purification may be a polyclonal or monoclonal antibody. Further, the protein of the present invention can be prepared by in vitro translation (see, for example, “On the fidelity of mRNA translation in the nuclease-treated rabbit reticulocyte lysate system. Dasso, M. C., Jackson, R. J. (1989) NAR 17:3129-3144”), or the like.

The present invention also provides DNAs encoding the above-mentioned proteins of the present invention. There is no limitation on the type of DNA of the present invention, as far as it can encode the protein of the present invention; comprising cDNA, genomic DNA, chemically synthesized DNA, etc. Further, when it encodes the protein of the present invention, a DNA having any nucleotide sequence based on the degeneration of genetic code is included. The DNA of the present invention can be isolated according to a standard method, such as the hybridization method using a DNA sequence encoding the GPRv protein (SEQ ID NOs: 5 to 8 and 22 to 26) or a partial sequence thereof as a probe or PCR method using primers synthesized based on these DNA sequence, as described above.

In addition, the present invention also provides a vector, in which the DNA of the present invention has been inserted. There is no limitation on the type of vector of the present invention, as far as it stably retains the inserted DNA. For example, when E. coli is used as a host, the preferable cloning vector is pBluescript vector (Stratagene) or the like. When the vector is used for the purpose of producing the protein of the present invention, an expression vector is especially useful. There is no limitation on the type of expression vector, as far as it direct the expression of the protein in vitro, in E. coli, in culture cells, in the living body, for example, pBEST (Promega) for in vitro expression; pET (Invitrogen) for in E. coli, ; pME18S-FL3 (GenBank Accession No. AB009864) for in culture cells; and, pME18S (Mol Cell Biol. 8:466-472(1988)) for in the living body of an organism are preferred vector. The insertion of the DNA of the present invention into a vector can be achieved according to a standard method, for example, by ligation using restriction enzyme sites (Current protocols in Molecular Biology, edit. Ausubel et al. (1987) Publish. John Wiley & Sons. Section 11.4-11.11).

Also, the present invention provides a transformant containing the DNA of the present invention or the vector of the present invention. There is no limitation on the type of host cell into which the vector of the present invention is to be introduced, and various types of host cells can be used depending on the purposes. Exemplary eukaryotic cells, in which the protein is to be expressed at high levels, include COS cell and CHO cell. The vector can be introduced into the host cell, by a known method such as, for example, calcium-phosphate precipitation method, electroporation method, (Current protocols in Molecular Biology, edit. Ausubel et al. (1987) Publish. John Wiley & Sons. Section 9.1-9.9), a method with lipofectamine (GIBCO-BRL), microinjection, and so forth.

The present invention also provides nucleotides comprising at least 15 nucleotide residues, which is complementary to the DNA encoding the protein of the present invention (DNA comprising any one of the nucleotide sequences of SEQ ID NOs: 5 to 8 and 22 to 26 or the complementary strand thereof). The term “complementary strand” means one strand complementary to the other strand of the two of double-stranded nucleic acid consisting of A:T (U in the case of RNA) and G:C nucleotide pairs. Further, the term “complementary” means not only being a perfect complementary sequence in a region of at least consecutive 15 nucleotide residues, but also nucleotide sequences with at least 70% homology, preferably at least 80%, more preferably 90%, further preferably 95% of homology or higher. The algorithm described herein can be used for determining homology. These nucleotides can be used as probes for detecting and isolating the DNA of the present invention, and as primers for amplifying the DNA of the present invention. When used as the primer, it typically comprises 15 bp -100 bp, preferably of 15 bp -35 bp of nucleotides. Alternatively, when used as the probe, it is at least 15 bp of nucleotide containing at least a part of the DNA of the present invention or the entire sequence. Preferably, such nucleotide specifically hybridize to the DNA encoding the protein of the present invention. The term “specifically hybridizing” means that a DNA hybridizes to the DNA encoding the protein of the present invention (SEQ ID NOs: 5 to 8 and 22 to 26) but not to DNAs encoding other proteins, under typical hybridization conditions, preferably under stringent conditions.

These nucleotides can be used for testing and diagnosing abnormalities of the protein of the present invention. For example, abnormal expression of the DNA encoding the protein of the present invention can be tested by Northern hybridization or RT-PCR using these nucleotides as probes or primers. The nucleotides can be used, for example, in the tests for cancers, cirrhosis, or Alzheimer's disease. In addition, the DNA encoding the protein of the present invention or the regulatory region for the expression is amplified by polymerase chain reaction (PCR) using the nucleotides as primers, and then abnormalities in the DNA sequence can be tested and diagnosed by using the methods such as RFLP analysis, SSCP, and sequencing.

Moreover, the antisense DNA for suppressing expression of the protein of present invention is included in these nucleotides. In order to cause the antisense effect, antisense DNA comprises at least 15 bp of nucleotides or more, preferably 100 bp, more preferably 500 bp or more, and usually comprises 3000 bp or less, preferably 2000 bp or less. Such antisense DNA may be applied to the gene therapy for the disease resulting from the abnormalities (abnormalities of function or expression) of the protein of present invention and so forth. This antisense DNA can be prepared, for example, based on the sequence information of DNA (for example, from SEQ ID NO: 5 to 8 and 22 to 26) encoding the protein of the present invention, by the phosphorothioate method (Stein, 1988 Physicochemical properties of phosphorothioate oligodeoxynucleotides. Nucleic Acids Res 16, 3209-21 (1988)), etc.

For gene therapy, the nucleotide of a present invention can be administered to a patient by ex vivo method, in vivo method, and so forth using virus vectors, such as a retrovirus vector, an adenovirus vector, and an adeno associated virus vector, and non-virus vectors, such as liposome, etc.

Further, the present invention provides the antibody bound with the protein of the present invention. There is no limitation in the form of the antibody of the present invention, and a polyclonal antibody and a monoclonal antibody, or a part thereof having antigen affinity are also included. Moreover, the antibody of all classes is included. Furthermore, the antibody of a present invention also include special antibodies, such as a humanized antibody.

For a polyclonal antibody, the antibody of the present invention can be obtained by synthesizing oligopeptides corresponding to the amino acid sequence of the protein of the present invention according to a standard, and then immunized to rabbit (Current protocols in Molecular Biology, edit. Ausubel et al. (1987) Publish. John Wiley & Sons. Section 11.12-11.13). For a monoclonal antibody, the hybridoma cell prepared by the cell fusion of the spleen cell and myeloma cell of the mouse immunized using the protein expressing in E. coli and then purified according to the standard method, and the antibody of the present invention can be obtained from this hybridoma cell (Current protocols in Molecular Biology, edit. Ausubel et al. (1987) Publish. John Wiley & Sons. Section 11.4-11.11).

In addition to purifying of the protein of the present invention, the antibody bound with the protein of the present invention may be also used for a test and a diagnosis of the abnormalities in expression or in structure of the protein of a present invention. Specifically, protein can be extracted from tissue, blood, or cell, and then can be used for the test and the diagnose for presence or absence of the abnormalities of expression or structure, via a detection of the protein of the present invention by Western blotting method, immunoprecipitation, ELISA, and so forth. The antibody of the present invention may be used for a test of cancer, liver cirrhosis, or Alzheimer's disease.

Moreover, the antibody bound with the protein of the present invention may be used for the purposes of, such as treatment of the disease relevant to the protein of the present invention. The antibody of the present invention can effect as the agonist and antagonist for the protein of the present invention. When using an antibody for the purpose of treatment of a patient, an antibody derived from human or a humanized antibody is preferable because of little immunogenicity. An antibody derived from human can be prepared by immunizing the mouse of which immune system is replaced with those of human (for example, refer to “Functional transplant of megabase human immunoglobulin loci recapitulates human antibody response in mice” Mendez, M. J. et al. (1997) Nat. Genet. 15: 146-156) Moreover, a humanized antibody can be prepared by recombination with the hypervariable region of a monoclonal antibody (Methods in Enzymology 203, 99-121 (1991)).

Further, the present invention also provides a screening method for ligands binding to the protein of the present invention using the protein of the present invention. This screening method comprises the step of: (a) contacting a test sample with the protein of the present invention or a partial peptide thereof; and (b) selecting compounds binding to the protein or the partial peptide thereof.

Without limiting, the test sample Include, for example, known compounds or peptides (for example, deposited in the Chemical File) whose activities as the ligands to the various G protein-coupled receptors have not yet been identified or a group of random peptides which have been prepared by phage display method (J. Mol. Biol. (1991) 222, 301-310). Further, culture supernatants of microorganisms, natural ingredients from plants or marine organisms, and, in addition to these, biological extracts from tissues including brain, cell extracts, expression products of gene libraries, but not limited thereto, can be screened.

The protein of the present invention to be used for the screening can be, for example, the form displayed on cell surface, the form as the cell membrane fraction of the cells, or the form immobilized in an affinity column.

Specific screening methods include many known methods such as, for example, a method of contacting a test sample with an affinity column of the protein of the present invention and purifying compounds bound to the protein of the present invention; and Western blotting method. When these methods are used, the test sample is labeled appropriately and the binding with the protein of the present invention can be detected by using the label. In addition to these methods, another method can be used; in which cell membranes expressing the protein of the present invention are prepared and immobilized on a chip, and the alterations in surface plasmon resonance, which represent the dissociation of the trimer-type GTP-binding protein during the ligand binding, are detected (Nature Biotechnology (99) 17:1105).

Further, the binding activity between a test sample and the protein of the present invention can be detected for alterations as indices in cells, which is caused by the binding of the test sample to the protein of the present invention expressed on cell surface. Such alterations include, for example, alterations of intracellular Ca²⁺ level and cAMP levels, but not limited thereto. Specifically, the agonist activity to the G protein-coupled receptor can be assayed by GTPγS binding method.

In an example where this method is used, cell membranes on which the G protein-coupled receptor has been displayed are mixed with 400 pM ³⁵S-labeled GTPγS in a solution containing 20 mM HEPES (pH 7.4), 100 mM NaCl, 10 mM MgCl₂, and 50 μM GDP, the mixture is incubated either in the presence or in absence of a test sample and then filtrated, and the radioactivities of the bound GTPγS are compared.

Further, the G protein-coupled receptors share the system of transducing signals into cells via the activation of the trimer-type GTP-binding protein. The trimer-type GTP-binding proteins are categorized into three classes depending on the types of activated systems of intracellular signal transduction: namely, Gq type that increases the Ca²⁺ level; Gs type that increases the cAMP level; and Gi type that reduces the cAMP. Thus, the use of a chimeric protein consisting of α-subunit from Gq protein and α-subunit from another type of G protein, or promiscuous Gα proteins, Gα15 and Gα16, allows the positive signal in the ligand screening to result in increased Ca²⁺ levels in the pathway of Gq intracellular signal transduction. The increased Ca²⁺ levels can be detected by using, as indices, altered levels of a reporter gene having TRE (TPA responsive element) or MRE (multiple responsive element) on upstream, dye indicator such as Fura-2 and Fluo-3, and fluorescent protein aequorin. Similarly, the use of a chimeric protein consisting of α-subunit from Gs protein and α-subunit from another type of G protein allows the positive signal to result in increased cAMP levels in the pathway of Gs intracellular signal transduction, and the increased levels can be detected by using, as indices, altered levels of a reporter gene having CRE (cAMP-responsive element) on upstream (Trends Pharmacol. Sci. (99) 20:118).

There is no limitation on the type of host cell to be used for the expression of the protein of the present invention in this screening system, and various types of host cells can be used depending on the purposes. Such host cells include, for example, COS cell, CHO cell, HEK293 cell, etc. The vectors directing the expression of the protein of the present invention in vertebrate cells, comprising the promoter upstream of the gene encoding the protein of the present invention, RNA splice site, polyadenylation site, and transcription termination sequence and replication origin, and so forth can be preferably used. For example, pSV2dhfr (Mol. Cell. Biol. (1981) 1, 854-864), pEF-BOS (Nucleic Acids Res. (1990) 18, 5322), pCDM8 (Nature (1987) 329, 840-842), and pCEP4 (Invitrogen), containing the SV40 early promoter, are useful vectors for the expression of the G protein-coupled receptor. The insertion of the DNA of the present invention into a vector can be achieved according to a standard method by ligation using restriction enzyme sites (Current protocols in Molecular Biology, edit. Ausubel et al. (1987) Publish. John Wiley & Sons. Section 11.4-11.11). Further, the vector introduction into a host cell can be achieved by a known method, for example, such as calcium-phosphate precipitation method, electroporation method (Current protocols in Molecular Biology, edit. Ausubel et al. (1987) Publish. John Wiley & Sons. Section 9.1-9.9), a method with lipofectamine (GIBCO-BRL), a method with FuGENE6 reagent (Boehringer-Manheim), microinjection method, etc.

Once the ligands are isolated by the above screening method for ligands binding to the protein of the present invention, screening of compounds inhibiting the interaction between the protein of the present invention and the ligands can be achieved. Thus, the present invention provides a screening method for compounds having the activity of inhibiting the binding of the protein of the present invention and the ligand thereof. This screening method comprises the step of: (a) contacting the ligand with the protein of the present invention or a partial peptide thereof in the presence of a test sample, and detecting the binding activity of the protein or a partial peptide thereof with the ligand; and (b) selecting compounds reducing the binding activity detected in the step (a) relative to the binding activity in the absence of the test sample.

Without limiting, the test sample include, for example, a group of compounds obtained by combinatorial chemistry technology (Tetrahedron (1995) 51, 8135-8137), a group of random peptides prepared by phage display method (J. Mol. Biol. (1991) 222, 301-310), and such. Further, culture supernatants of microorganisms and natural ingredients from plants or marine organisms, and in addition to these, biological extracts from tissues including brain, cell extracts, expression products of gene libraries, synthetic low-molecular-weight compounds, synthetic peptides, natural compounds, and so forth can be screened, but not limited thereto.

The protein of the present invention to be used for the screening can be, for example, the form expressed on cell surface, the form in the cell membrane fraction of the cells, or the form immobilized in an affinity column.

Specific methods that can be used for the screening include, for example, a method in which the ligand is labeled with a radioisotope or the like, and contacted with the protein of the present invention in the presence of a test sample, and then, based on the label linked to the ligand, compounds reducing the binding activity of the protein of the present invention to the ligand are detected as compared to those detected in the. absence of the test sample. Further, the screening can also be achieved by using the intracellular alterations as an index by the same method as used in the above-mentioned screening to isolate ligands capable of binding to the protein of the present invention. Specifically, the screening for a compound inhibiting the binding of the protein of the present invention with the ligand can be carried out by contacting cells expressing the protein of the present invention with the ligands in the presence of a test sample, and selecting a compound decreasing the degree of alteration in the cells as compared with those detected in the absence of the test sample. The cells expressing the protein of the present invention can be prepared by the same method as used in the above-described screening of ligands binding to the protein of the present invention. The compounds isolated by the screening can be candidates for the agonist or antagonist to the protein of the present.

Further, the present invention provides a screening method for compounds inhibiting or enhancing the activity of the protein of the present invention. This screening method comprises the step of: (a) contacting the ligand to the protein with cells expressing the protein of the present invention in the presence of a test sample; (b) detecting an alteration in the cells due to the binding of the ligand to the protein of the present invention; and (c) selecting compounds suppressing or enhancing the alteration in the cells detected in the step (b) as compared with the alteration of the cells in the absence of the test sample.

Such test samples to be used, like those to be used in the above-mentioned screening method for ligands binding to the protein of the present invention, include a group of compounds obtained by combinatorial chemistry technology, a group of random peptides prepared by using phage display method, culture supernatants of microorganisms, natural ingredients from plants or marine organisms, biological tissue extracts, cell extracts, expression products of gene libraries, synthetic low-molecular-weight compounds, synthetic peptides, natural compounds, and such. Further, the compounds isolated by the above-mentioned screening of ligands binding to the protein of the present invention can be used as the test samples. The cells expressing the protein of the present invention can be prepared by the same method as the above-described screening of ligands binding to the protein of the present invention. The alteration in the cells after contacted with the test sample can be detected by using the alteration of intracellular Ca²⁺ level or cAMP level as an index, as with the above screening method. Further, the intracellular signal transduction can also be detected by using an assay system such as a reporter assay using luciferase as a reporter gene.

When the result of the detection shows that the alteration in the cells contacted with a test sample is suppressed as compared to those in the cells contacted with the ligand in the absence of the test sample, the test sample used is determined to be a compound inhibiting the activity of the protein of the present invention. Conversely, when the test sample enhances the alteration in the cells, the compound is determined to be a compound enhancing the activity of the protein of the present invention. The term “enhancing or inhibiting the activity of protein of the present invention” means that, regardless of a direct or an indirect interaction to the protein of the present invention, the interaction results in the enhancement or inhibition of the activity of protein of the present invention. Accordingly, the compounds isolated by the screening include compounds acting on the protein of the present invention or the ligand and inhibiting or enhancing the activity of the protein of the present invention by inhibiting or enhancing the binding, as well as compounds which do not inhibit nor enhance the binding itself but result in the inhibition or enhancement of the activity of the protein of the present invention. Such compounds include, for example, compounds which do not inhibit nor enhance the binding of the protein of the present invention and the ligand but inhibit or enhance the pathway of intracellular signal transduction.

When the compounds isolated by the screening method of the present invention are used as pharmaceuticals, the isolated compound not only can be directly administered itself to patients but also can be administered as pharmaceutical compositions which have been formulated by a known pharmaceutical method. For example, the compound can be formulated, in a form suitable for oral or parenteral administration, as a pharmaceutical composition obtained by combining the compound with pharmaceutically acceptable carrier (for example, excipient, binder, disintegrator, flavor, corrigent, emulsifier, diluent, solubilizer, etc.), or preparations, such as tablet, pill, powder, granule, capsule, troche, syrup, liquid drug, emulsion, suspension, injection (e.g. liquid drug and suspension), suppository, inhalant, percutaneous absorbent, eye drop, eye ointment, and so forth,. In general, the administration to patients can be carried out by a method known to those skilled in the art, including intraarterial injection, intravenous injection, subcutaneous injection, etc. While the doses are different depending on the weight and age of patient, administration method, and such, those skilled in the art can chose proper administration doses if necessary. Further, when the compound is encoded by a DNA, the DNA can be inserted into a vector for gene therapy and thus can be used for gene therapy. The compound isolated by the screening method of the present invention is expected to be applied to the treatment of, for example, cancers, cirrhosis, and Alzheimer's disease.

The present invention also provides a disease diagnosing method for cancers, cirrhosis, or Alzheimer's disease, comprising the step of detecting the expression of the gene encoding the GPRv protein of the present invention.

In the Example herein, it has been found that the expression levels of the genes encoding the GPRV proteins of the present invention in affected tissues associated with cancers, cirrhosis, or Alzheimer's disease are significantly different as compared to those in normal tissues. Thus, these diseases can be diagnosed by detecting the expression of the genes encoding the GPRv proteins of the present invention in tissues of subjects. The term “gene expression” means both transcription and translation.

The diagnosis method of the present invention can be carried out, for example, as follows.

The diagnosis can be achieved by extracting RNA from an aliquot of a tissue collected by biopsy or blood sample according to a standard method, and quantifying GPRv mRNA by quantitative PCR, Northern hybridization, or dot blot hybridization, and such, as described in the Example herein. Alternatively, the diagnosis can also be achieved by quantifying the GPRv protein in a protein extract from the above tissue by a method such as Western blotting, immunoprecipitation, ELISA, and such, or by a noninvasive method where a labeled compound or antibody binding to the GPRv protein is administered to patients and detected by PET (positron emission tomography) or the like.

When the result of the diagnosis shows that the gene expression in the tissues of a subject exhibits a pattern (for example, an increased or decreased gene expression level as compared to that in the normal tissue) identical to that of the gene expression in the tissue derived from a patient affected with any one of the above diseases, the subject is determined as being affected or as being at a risk of affection with the disease.

For example, the expression of GPRv8 was detectable in the colon, and the expression level was markedly higher in colon cancers. Accordingly, when the expression of GPRv8 is detected at a high level in the colon tissue of a subject, the subject is suspected of colon cancer. Alternatively, the expression of GPRv8 was undetectable in the normal pancreas and uterus, but GPRv8 was expressed at a moderate level after canceration. Accordingly, when the expression of GPRv8 can be detected in the pancreas or uterus of a subject, the subject is suspected of pancreatic cancer or uterine cancer.

The expression of GPRv12 was undetectable in the normal ovary and testis, but was detectable after canceration. Further, the expression level decreased in the hippocampus with Alzheimer's disease. Accordingly, when the expression of GPRv12 is detected in the ovary or testis of a subject, the subject is suspected of ovary cancer or testicular cancer. Similarly, when the expression of GPRv12 is detected in the hippocampus of a subject at a lower level than the normal level, the subject is suspected of Alzheimer's disease.

GPRv16 was expressed in the colon, but was undetectable after canceration. The expression level increased in the brain after canceration. In the liver, the expression was undetectable after cirrhosis. In the brain of patients with Alzheimer's disease, the expression level was elevated at the hippocampus. Accordingly, when the expression of GPRv16 is detected in the colon of a subject at a lower level than the normal level, the subject is suspected of colon cancer. Further, when the expression is detected in the brain at a higher level than the normal level, the subject is suspected of brain cancer. Further, the expression of GPRv16 is detected in the liver at a lower level than the normal level, the subject is suspected of cirrhosis. Further, when the expression of GPRv16 is detected in the hippocampus at a higher level than the normal level, the subject is suspected of Alzheimer's disease.

The expression of GPRv21 was undetectable in the colon and testis after canceration. Accordingly, when the expression of GPRv21 is detected in the colon or testis of a subject at a lower level than the normal level, the subject is suspected of colon cancer or testicular cancer.

The expression level of GPRv40 increased in the brain and testis after canceration, and decreased in the liver after cirrhosis. Accordingly, when the expression of GPRv40 is detected in the brain or testis at a higher level than the normal level, the subject is suspected of brain tumor or testicular cancer. Further, when the expression of GPRv40 was detected in the liver at a lower level than the normal level, the subject is suspected of cirrhosis.

The expression level of GPRv47 increased in the brain and kidney and decreased in the testis, after canceration. The expression was undetectable in the liver after cirrhosis. Accordingly, when the expression of GPRv47 is detected in the brain or kidney at a higher level than the normal level, the subject is suspected of brain tumor or kidney cancer. Further, when the expression of GPRv47 is detected in the liver at a lower level than the normal level, the subject is suspected of cirrhosis.

The expression level of GPRv51 decreased in the colon and testis after canceration. The expression level also decreased in the liver after cirrhosis as compared to the normal liver. The expression level increased in the hippocampus with Alzheimer's disease. Accordingly, when the expression of GPRv51 is detected in the colon and testis at a lower level than the normal level, the subject is suspected of colon cancer or testicular cancer. Further, when the expression of GPRv51 is detected in the liver at a lower level than the normal level, the subject is suspected of cirrhosis. Further, when the expression of GPRv51 is detected in the hippocampus at a higher level than the normal level, the subject is suspected of Alzheimer's disease.

The expression level of GPRv71 decreased in the colon and kidney, and was undetectable in the liver, after cirrhosis. In Alzheimer's disease, the expression level decreased in the frontal lobe. Accordingly, when the expression of GPRv71 is detected in the colon or kidney at a lower level than the normal level, the subject is suspected of colon cancer or kidney cancer. Further, when the expression of GPRv71 is detected in the liver at a lower level than the normal level, the subject is suspected of cirrhosis. Further, when the expression of GPRv71 is detected in the frontal lobe at a lower level than the normal level, the subject is suspected of Alzheimer's disease.

GPRv72 was expressed strongly in the colon, but the expression was undetectable after canceration. The expression level of GPRv72 increased in the hippocampus with Alzheimer's disease. Accordingly, when the expression of GPRv72 is detected in the colon at a lower level than the normal level, the subject is suspected of colon cancer. Further, when the expression of GPRv72 is detected in the hippocampus at a higher level than the normal level, the subject is suspected of Alzheimer's disease.

Furthermore, mutations in the genes encoding GPRv proteins of the present invention may result in the onset of the above-mentioned diseases. Thus, the diagnosis for the above-mentioned diseases can be carried out by detecting such mutations in the genes encoding GPRv proteins of the present invention.

Such gene diagnosis can be carried out, for example, as follows.

As a nucleic acid to be used for the diagnosis, genomic DNA or cDNA may be amplified directly or by PCR or other amplification technique. Deletions and insertions can be detected based on size differences of the amplification products as compared with that of the normal gene. Point mutations can be identified based on the differences in the melting temperature of the amplified DNA hybridized with DNA encoding GPRv. Differences between DNA sequences can be found by detecting alterations in the electrophoretic mobility of DNA fragment in a denaturant-containing or denaturant-free gel or by direct sequencing of nucleotide sequence of DNA.

When the diagnosis result shows that the gene encoding the GPRv protein from a subject has mutations as compared with the wild-type sequence, the subject diagnosed to be suspected of the above disease.

Namely, a method for diagnosing cancers, cirrhosis, or Alzheimer's disease or a method for diagnosing the susceptibility to the diseases are provided by detecting, according to the method described herein, mutations in the genes encoding the GPRv proteins or increase or decrease in the expression levels of the mRNAs or proteins.

### BRIEF DESCRIPTION OF THE DRAWINGS

FIG. 1 shows a result of BLAST SEARCH with the “GPRv8” amino acid sequence as the query against the entire sequence data in SWISS-PROT. The sequence showed 36% homology to HUMAN VASOPRESSIN V1B RECEPTOR.

FIG. 2 shows a result of BLAST SEARCH with the “GPRv12” amino acid sequence as the query against the entire sequence data in SWISS-PROT. The sequence showed 27% homology to RAT 5-HYDROXYTRYPTAMINE 6 RECEPTOR.

FIG. 3 shows a result of BLAST SEARCH with the “GPRv16” amino acid sequence as the query against the entire sequence data in SWISS-PROT. The sequence showed 28% homology to MOUSE GALANIN RECEPTOR TYPE 1.

FIG. 4 shows a result of BLAST SEARCH with the “GPRv21” amino acid sequence as the query against the entire sequence data in SWISS-PROT. The sequence showed 30% homology to BOVIN NEUROPEPTIDE Y RECEPTOR TYPE 2.

FIG. 5 shows a result of BLAST SEARCH with the “GPRv40” amino acid sequence as the query against the entire sequence data in SWISS-PROT. The sequence showed 34% homology to OXYTOCIN RECEPTOR (P97926).

FIG. 6 shows a result of BLAST SEARCH with the “GPRv47” amino acid sequence as the query against the entire sequence data in SWISS-PROT. The sequence showed 43% homology to GPRX_ORYLA PROBABLE G PROTEIN-COUPLED RECEPTOR (Q91178).

FIG. 7 shows a result of BLAST SEARCH with the “GPRv51” amino acid sequence as the query against the entire sequence data in SWISS-PROT. The sequence showed 37% homology to PROBABLE G PROTEIN-COUPLED RECEPTOR RTA (P23749).

FIG. 8 shows a result of BLAST SEARCH with the “GPRv71” amino acid sequence as the query against the entire sequence data in SWISS-PROT. The sequence showed 45% homology to P2Y PURINOCEPTOR 3 (P2Y3) (Q98907).

FIG. 9 shows a result of BLAST SEARCH with the “GPRv72” amino acid sequence as the query against the entire sequence data in SWISS-PROT. The sequence showed 30% homology to ALPHA-1A ADRENERGIC RECEPTOR (O02824).

FIG. 10 shows a hydropathy plot for GPRv8.

FIG. 11 shows an alignment of GPRv8 and similar families. The mark ‘*’ means that the amino acid is completely conserved in all the sequences at the position marked therewith. The mark ‘:’ means that amino acids at the position marked therewith are conserved within any one of the following groups: {STA}, {NEQK}, {NHQK}, {NDBQ}, {QHRK}, {MILV}, {MILF}, {HY}, and {FYW}. The mark ‘.’ means that amino acids at the position marked therewith are conserved within anyone of the following groups: {CSA}, {ATV}, {SAG}, {STNK}, {STPA}, {SGND}, {SNDEQK}, {NDEQHK}, and {NEQHRK}.

FIG. 12 is continued from FIG. 11.

FIG. 13 shows a hydropathy plot for GPRv12.

FIG. 14 shows an amino acid sequence alignment of GPRv12 and AF208288. The mark ‘*’ means that the amino acid is completely conserved in all the sequences at the position marked therewith. The mark ‘:’ means that amino acids at the position marked therewith are conserved within any one of the following groups: {STA}, {NEQK}, {NHQK}, {NDBQ}, {QHRK}, {MILV}, {MILF}, {HY}, and {FYW}. The mark ‘.’ means that amino acids at the position marked therewith are conserved within any one of the following groups: {CSA}, {ATV}, {SAG}, {STNK}, {STPA}, {SGND}, {SNDEQK}, {NDEQHK}, and {NEQHRK}.

FIG. 15 shows a hydropathy plot for GPRv16.

FIG. 16 shows a summary of HMMPFAM, transmembrane domain, and S—S bond of GPRv16. The mark “***” indicates a region assigned as 7tm_{—}1 based on the result of HMMPFAM. The mark “###” represents transmembrane domain. The mark “@” indicates Cys capable of forming S—S bond.

FIG. 17 shows a hydropathy plot for GPRv21.

FIG. 18 shows an amino acid sequence alignment of GPRv21 and the related proteins. The mark ‘*’ means that the amino acid is completely conserved in all the sequences at the position marked therewith. The mark ‘:’ means that amino acids at the position marked therewith are conserved within any one of the following groups: {STA}, {NEQK}, {NHQK}, {NDBQ}, {QHRK}, {MILV}, {MILF}, {HY}, and {FYW}. The mark ‘.’ means that amino acids at the position marked therewith are conserved within any one of the following groups: {CSA}, {ATV}, {SAG}, {STNK}, {STPA}, {SGND}, {SNDEQK}, {NDEQHK}, and {NEQHRK}.

FIG. 19 is continued from FIG. 18.

FIG. 20 shows a hydropathy plot for GPRv40.

FIG. 21 shows a summary of HMMPFAM, transmembrane domain, and S—S bond of GPRv40. The mark “***” indicates a region assigned as 7tm_{—}1 based on the result of HMMPFAM. The mark “###” indicates transmembrane domain. The mark “@” indicates Cys capable of forming S—S bond.

FIG. 22 shows a hydropathy plot for GPRv47.

FIG. 23 shows an alignment of GPRv47 and the related proteins. The mark ‘*’ means that the amino acid is completely conserved in all the sequences at the position marked therewith. The mark ‘:’ means that amino acids at the position marked therewith are conserved within any one of the following groups: {STA}, {NEQK}, {NHQK}, {NDBQ}, {QHRK}, {MILV}, {MILF}, {HY}, and {FYW}. The mark ‘.’ means that amino acids at the position marked therewith are conserved within any one of the following groups: {CSA}, {ATV}, {SAG}, {STNK}, {STPA}, {SGND}, {SNDEQK}, {NDEQHK}, and {NEQHRK}.

FIG. 24 is continued from FIG. 23.

FIG. 25 is continued from FIG. 24.

FIG. 26 shows a hydropathy plot for GPRv51.

FIG. 27 shows an alignment of GPRv51 and the related proteins. The mark ‘*’ means that the amino acid is completely conserved in all the sequences at the position marked therewith. The mark ‘:’ means that amino acid at the position marked therewith are conserved within any one of the following groups: {STA}, {NEQK}, {NHQK}, {NDBQ}, {QHRK}, {MILV}, {MILF}, {HY}, and {FYW}. The mark ‘.’ means that amino acid at the position marked therewith are conserved within any one of the following groups: {CSA}, {ATV}, {SAG}, {STNK}, {STPA}, {SGND}, {SNDEQK}, {NDEQHK}, and {NEQHRK}.

FIG. 28 shows a hydropathy plot for GPRv71.

FIG. 29 shows an alignment of GPRv71 and related proteins. The mark ‘*’ means that the amino acid is completely conserved in all the sequences at the position marked therewith. The mark ‘:’ means that amino acid at the position marked therewith are conserved within any one of the following groups: {STA}, {NEQK}, {NHQK}, {NDBQ}, {QHRK}, {MILV}, {MILF}, {HY}, and {FYW}. The mark ‘.’ means that amino acid at the position marked therewith are conserved within any one of the following groups: {CSA}, {ATV}, {SAG}, {STNK}, {STPA}, {SGND}, {SNDEQK}, {NDEQHK}, and {NEQHRK}.

FIG. 30 is continued from FIG. 29.

FIG. 31 shows a hydropathy plot for GPRv72.

FIG. 32 shows an alignment of GPRv72 and related proteins. The mark ‘*’ means that the amino acid is completely conserved in all the sequences at the position marked therewith. The mark ‘:’ means that amino acid at the position marked therewith are conserved within any one of the following groups: {STA}, {NEQK}, {NHQK}, {NDBQ}, {QHRK}, {MILV}, {MILF}, {HY}, and {FYW}. The mark ‘.’ means that amino acid at the position marked therewith are conserved within any one of the following groups: {CSA}, {ATV}, {SAG}, {STNK}, {STPA}, {SGND}, {SNDEQK}, {NDEQHK}, and {NEQHRK}.

FIG. 33 is continued from FIG. 32.

FIG. 34 is continued from FIG. 33.

### BEST MODE FOR CARRYING OUT THE INVENTION

The present invention is specifically illustrated below with reference to Examples, but it is not to be construed as being limited thereto. Unless otherwise stated, they can be carried out by known methods (Maniatis, T. et al. (1982): “Molecular Cloning—A Laboratory Manual”, Cold Spring Harbor Laboratory, NY).

### EXAMPLE 1

### Isolation of the Genes Encoding the Novel G Protein-coupled Receptors

The full-length cDNAs encoding the novel G protein-coupled receptors of the present invention (GPRv8, GPRv12, GPRv16, GPRv21, GPRv40, GPRv47, GPRv51, GPRv71, and GPRv72) were obtained by PCR.

The amplification of the novel G protein-coupled receptor GPRv8 was carried out using a Marathon Ready cDNA (Clontech) derived from human fetus as a template, and forward primer: 5′-ATGCCAGCCAACTTCACAGAGGGCAGCT-3′ (SEQ ID NO: 9) and reverse primer: 5′-CTAGATGAATTCTGGCTTGGACAGAATC-3′ (SEQ ID NO: 10). PCR was carried out with Pyrobest DNA polymerase (Takara); the thermal cycling profile consisted of preheat at 94° C. (2.5 minutes) and 25 cycles of 94° C. (30 seconds)/60° C. (30 seconds)/72° C. (1 minute). The amplification resulted in about 1.1-kbp DNA fragments. The fragments were cloned into pCR2.1 plasmid (Invitrogen). The nucleotide sequence of the resultant clone was determined by dideoxy terminator method in an ABI377 DNA Sequencer (Applied Biosystems). The determined sequence is shown in SEQ ID NO: 5.

The sequence comprises an open reading frame of 1116 nucleotides (from the first nucleotide to the 1116th nucleotide in SEQ ID NO: 5). An amino acid sequence deduced from the open reading frame (371 amino acids) is shown in SEQ ID NO: 1. Since the deduced amino acid sequence contains hydrophobic regions corresponding to seven transmembrane domains characteristic of G protein-coupled receptor, the gene is found to encode a G protein-coupled receptor.

The amplification of the novel G protein-coupled receptor GPRv12 was carried out using a Marathon Ready cDNA (Clontech) derived from human fetal brain as a template, and forward primer: 5′-ATGGGCCCCGGCGAGGCGCTGCTGGCGG-3′ (SEQ ID NO: 11) and reverse primer: 5′-TCAGTGTGTCTGCTGCAGGCAGGAATCA-3′ (SEQ ID NO: 12). PCR was carried out with Pyrobest DNA polymerase (Takara) under the presence of 5% formamide; the thermal cycling profile consisted of preheat at 94° C. (2.5 minutes), 5 cycles of 94° C. (5 seconds)/72° C. (4 minutes), 5 cycles of 94° C. (5 seconds)/70° C. (4 minutes), and 25 cycles of 94° C. (5 seconds)/68° C. (4 minutes). The amplification resulted in about 1.1-kbp DNA fragments. The fragments were cloned into pCR2.1 plasmid (Invitrogen). The nucleotide sequence of the resultant clone was determined by dideoxy terminator method in an ABI377 DNA Sequencer (Applied Biosystems). The determined sequence is shown in SEQ ID NO: 6.

The sequence comprises an open reading frame of 1092 nucleotides (from the first nucleotide to the 1092th nucleotide in SEQ ID NO: 6). An amino acid sequence deduced from the open reading frame (363 amino acids) is shown in SEQ ID NO: 2. Since the deduced amino acid sequence contains hydrophobic regions corresponding to seven transmembrane domains characteristic of G protein-coupled receptor, the gene is found to encode a G protein-coupled receptor.

The amplification of the novel G protein-coupled receptor GPRv16 was carried out using a Marathon Ready cDNA (Clontech) derived from human brain as a template, and forward primer: 5′-ATGCTGGCAGCTGCCTTTGCAGACTCTAAC-3′ (SEQ ID NO: 13) and reverse primer: 5′-CTATTTAACACCTTCCCCTGTCTCTTGATC-3′ (SEQ ID NO: 14). PCR was carried out with Pyrobest DNA polymerase (Takara); the thermal cycling profile consisted of preheat at 94° C. (2 minutes) and 30 cycles of 94° C. (30 seconds)/60° C. (30 seconds)/72° C. (1 minute) The amplification resulted in about 1.2-kbp DNA. fragments. The fragments were cloned into pCR2.1 plasmid (Invitrogen). The nucleotide sequence of the resultant clone was determined by dideoxy terminator method in an ABI377 DNA Sequencer (Applied Biosystems). The determined sequence is shown in SEQ ID NO: 7.

The sequence comprises an open reading frame of 1260 nucleotides (from the first nucleotide to the 1260th nucleotide in SEQ ID NO: 7). An amino acid sequence deduced from the open reading frame (419 amino acids) is shown in SEQ ID NO: 3. Since the deduced amino acid sequence contains hydrophobic regions corresponding to seven transmembrane domains characteristic of G protein-coupled receptor, the gene is found to encode a G protein-coupled receptor.

The amplification of the novel G protein-coupled receptor GPRv21 was carried out using a Marathon Ready cDNA (Clontech) derived from human fetus as a template, and forward primer: 5′-ATGGAGACCACCATGGGGTTCATGGATG-3′ (SEQ ID NO: 15) and reverse primer: 5′-TTATTTTAGTCTGATGCAGTCCACCTCTTC-3′ (SEQ ID NO: 16). PCR was carried out with Pyrobest DNA polymerase (Takara) under the presence of 5% formamide; the thermal cycling profile consisted of preheat at 94° C. (2.5 minutes), 5 cycles of 94° C. (5 seconds)/72° C. (4 minutes), 5 cycles of 94° C. (5 seconds)/70° C. (4 minutes), and 25 cycles of 94° C. (5 seconds)/68° C. (4 minutes). The amplification resulted in about 1.2-kbp DNA fragments. The fragments were cloned into pCR2.1 plasmid (Invitrogen). The nucleotide sequence of the resultant clone was determined by dideoxy terminator method in an ABI377 DNA Sequencer (Applied Biosystems). The determined sequence is shown in SEQ ID NO: 8.

The sequence comprises an open reading frame of 1182 nucleotides. An amino acid sequence deduced from the open reading frame (333 amino acids) is shown in SEQ ID NO: 4. Since the deduced amino acid sequence contains hydrophobic regions corresponding to seven transmembrane domains characteristic of G protein-coupled receptor, the gene is found to encode a G protein-coupled receptor.

The amplification of the novel G protein-coupled receptor GPRv40 was carried out using a Marathon Ready cDNA (Clontech) derived from human fetus as a template, and forward primer: 5′-ATGGAGGATCTCTTTAGCCCCTCAATTC-3′ (SEQ ID NO: 27) and reverse primer: 5′-CTAGAAGGCACTTTCGCAGGAGCAAGGC-3′ (SEQ ID NO: 28). PCR was carried out with Pyrobest DNA polymerase (Takara) under the presence of 5% formamide; the thermal cycling profile consisted of preheat at 98° C. (2.5 minutes), 5 cycles of 98° C. (5 seconds)/72° C. (4 minutes) , 5 cycles of 98° C. (5 seconds)/70° C. (4 minutes) , and 25 cycles of 98° C. (5 seconds)/68° C. (4 minutes). The amplification resulted in about 1.3-kbp DNA fragments. The fragments were cloned into pCR2.1 plasmid (Invitrogen). The nucleotide sequence of the resultant clone was determined by dideoxy terminator method in an ABI377 DNA Sequencer (Applied Biosystems). The determined sequence is shown in SEQ ID NO: 22.

The sequence comprises an open reading frame of 1305 nucleotides (SEQ ID NO: 22). An amino acid sequence deduced from the open reading frame (434 amino acids) is shown in SEQ ID NO: 17. Since the deduced amino acid sequence contains hydrophobic regions corresponding to seven transmembrane domains characteristic of G protein-coupled receptor, the gene is found to encode a G protein-coupled receptor.

The amplification of the novel G protein-coupled receptor GPRv47 was carried out using a Marathon Ready cDNA (Clontech) derived from human fetal brain as a template, and forward primer: 5′-ATGGAGTCCTCACCCATCCCCCAGTCATC-3′ (SEQ ID NO: 29) and reverse primer: 5′-TCATGACTCCAGCCGGGGTGAGGCGGCAG-3′ (SEQ ID NO: 30). PCR was carried out with Pyrobest DNA polymerase (Takara) under the presence of 5% formamide; the thermal cycling profile consisted of preheat at 94° C. (2 minutes) and 35 cycles of 94° C. (30 seconds)/50° C. (30 seconds)/72° C. (1.5 minutes). The amplification resulted in about 1.4-kbp DNA fragments. The fragments were cloned into pCR2.1 plasmid (Invitrogen). The nucleotide sequence of the resultant clone was determined by dideoxy terminator method in an ABI377 DNA Sequencer (Applied Biosystems). The determined sequence is shown in SEQ ID NO: 23.

The sequence comprises an open reading frame of 1356 nucleotides (SEQ ID NO: 23). An amino acid sequence deduced from the open reading frame (451 amino acids) is shown in SEQ ID NO: 18. Since the deduced amino acid sequence contains hydrophobic regions corresponding to seven transmembrane domains characteristic of G protein-coupled receptor, the gene is found to encode a G protein-coupled receptor.

The amplification of the novel G protein-coupled receptor GPRv51 was carried out using a Marathon Ready cDNA (Clontech) derived from human testis as a template, and forward primer: 5′-ATGAACCAGACTTTGAATAGCAGTGG-3′ (SEQ ID NO: 31) and reverse primer: 5′-TCAAGCCCCCATCTCATTGGTGCCCACG-3′ (SEQ ID NO: 32). PCR was carried out with Pyrobest DNA polymerase (Takara); the thermal cycling profile consisted of preheat at 98° C. (2.5 minutes) and 35 cycles of98° C. (30 seconds)/50° C. (30 seconds)/68° C. (4minutes). The amplification resulted in about 1.0-kbp DNA fragments. The fragments were cloned into pCR2.1 plasmid (Invitrogen). The nucleotide sequence of the resultant clone was determined by dideoxy terminator method in an ABI377 DNA Sequencer (Applied Biosystems). The determined sequence is shown in SEQ ID NO: 24.

The sequence comprises an open reading frame of 966 nucleotides (SEQ ID NO: 24). An amino acid sequence deduced from the open reading frame (321 amino acids) is shown in SEQ ID NO: 19. Since the deduced amino acid sequence contains hydrophobic regions corresponding to seven transmembrane domains characteristic of G protein-coupled receptor, the gene is found to encode a G protein-coupled receptor.

The amplification of the novel G protein-coupled receptor GPRv71 was carried out using a Marathon Ready cDNA (Clontech) derived from human fetus as a template, and forward primer: 5′-ATGGAGAAGGTGGACATGAATACATCAC-3′ (SEQ ID NO: 33) and reverse primer: 5′-TTACCCAGATCTGTTCAACCCTGGGCATC-3′ (SEQ ID NO: 34). PCR was carried out with Pyrobest DNA polymerase (Takara); the thermal cycling profile consisted of preheat at 94° C. (2.5 minutes) , 5 cycles of 98° C. (5 seconds)/72° C. (4 minutes), 5 cycles of 98° C. (5 seconds)/70° C. (4 minutes) , and 25 cycles of 98° C. (5 seconds)/68° C. (4 minutes). The amplification resulted in about 1.0-kbp DNA fragments. The fragments were cloned into pCR2.1 plasmid (Invitrogen). The nucleotide sequence of the resultant clone was determined by dideoxy terminator method in an ABI377 DNA Sequencer (Applied Biosystems). The determined sequence is shown in SEQ ID NO: 25.

The sequence comprises an open reading frame of 1002 nucleotides (SEQ ID NO: 25). An amino acid sequence deduced from the open reading frame (333 amino acids) is shown in SEQ ID NO: 20. Since the deduced amino acid sequence contains hydrophobic regions corresponding to seven transmembrane domains characteristic of G protein-coupled receptor, the gene is found to encode a G protein-coupled receptor.

The amplification of the novel G protein-coupled receptor GPRv72 was carried out using human genome DNA (Clontech) as a template, and forward primer: 5′-ATGACGTCCACCTGCACCAACAGCACGC-3′ (SEQ ID NO: 35) and reverse primer: 5′-TCAAGGAAAAGTAGCAGAATCGTAGGAAG-3′ (SEQ ID NO: 36). PCR was carried out with Pyrobest DNA polymerase (Takara); the thermal cycling profile consisted of preheat at 94° C. (2 minutes) and 30 cycles of94° C. (30 seconds)/55° C. (30 seconds)/68° C. (4 minutes). The amplification resulted in about 1.5-kbp DNA fragments. The fragments were cloned into pCR2.1 plasmid (Invitrogen). The nucleotide sequence of the resultant clone was determined by dideoxy terminator method in an ABI377 DNA Sequencer (Applied Biosystems). The determined sequence is shown in SEQ ID NO: 26.

The sequence comprises an open reading frame of 1527 nucleotides (SEQ ID NO: 26). An amino acid sequence deduced from the open reading frame (508 amino acids) is shown in SEQ ID NO: 21. Since the deduced amino acid sequence contains hydrophobic regions corresponding to seven transmembrane domains characteristic of G protein-coupled receptor, the gene is found to encode a G protein-coupled receptor.

### EXAMPLE 2

### BLAST SEARCH of the Amino Acid Sequences of the Novel G Protein-coupled Receptors Against SWISS-PROT

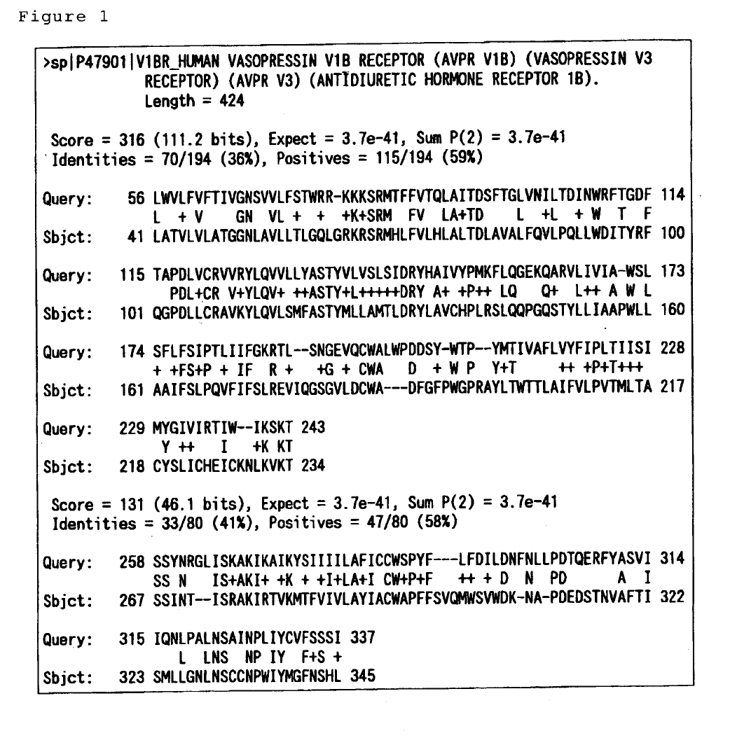

The result of BLAST SEARCH of the amino acid sequence of “GPRv8” against SWISS-PROT is shown in FIG. 1. “GPRv8” exhibited the highest homology (36%) to HUMAN VASOPRESSIN V1B RECEPTOR (P47901, 424 aa) of known G protein-coupled receptors. Thus, “GPRv8” was concluded to be a novel G protein-coupled receptor.

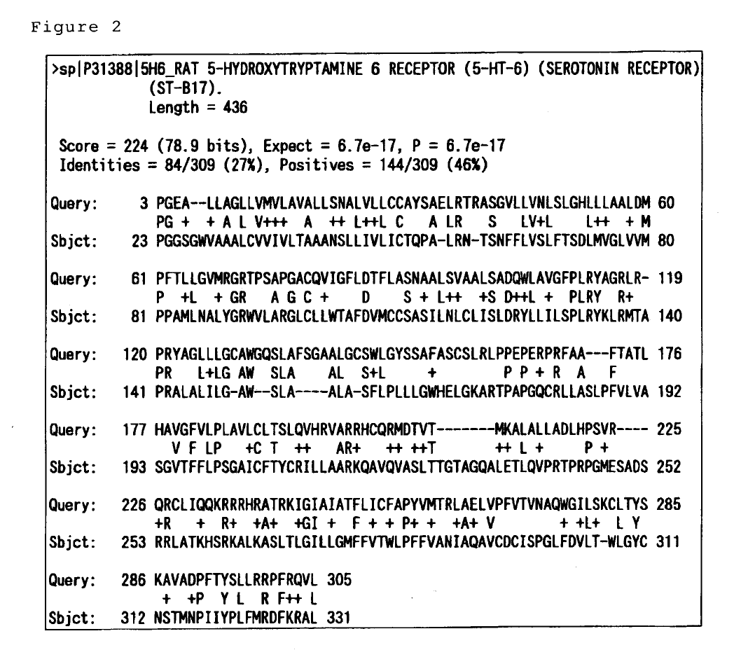

The result of BLAST SEARCH of the amino acid sequence of “GPRv12” against SWISS-PROT is shown in FIG. 2. “GPRv12” exhibited the highest homology (27%) to RAT 5-HYDROXYTRYPTAMINE 6 RECEPTOR (P31388, 436 aa) of known G protein-coupled receptors. Thus, GPRv12 was concluded to be a novel G protein-coupled receptor.

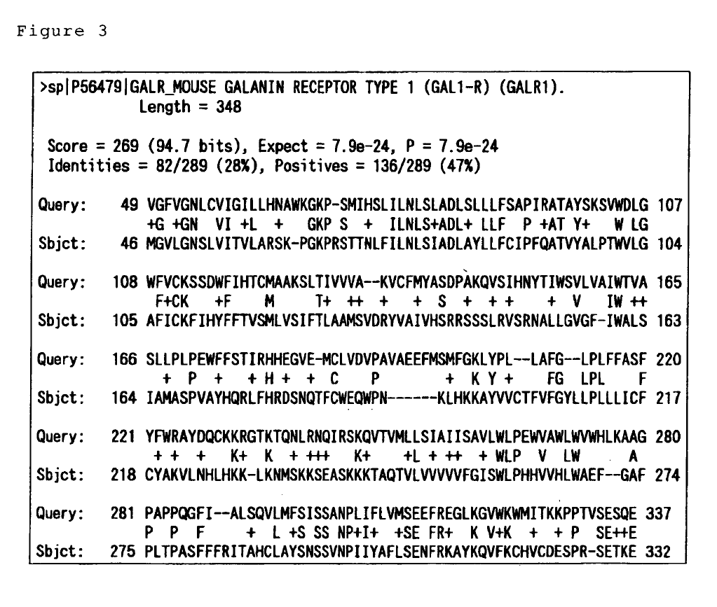

The result of BLAST SEARCH of the amino acid sequence of “GPRv16” against SWISS-PROT is shown in FIG. 3. “GPRv16” exhibited the highest homology (28%) to MOUSE GALANIN RECEPTOR TYPE 1 (P56479, 348 aa) of known G protein-coupled receptors. Thus, “GPRv16” was concluded to be a novel G protein-coupled receptor.

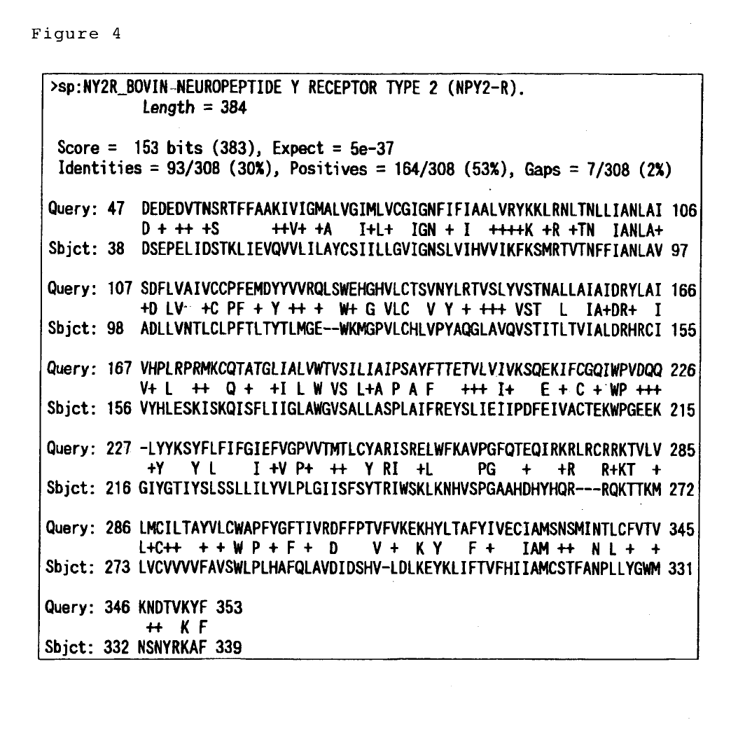

The result of BLAST SEARCH of the amino acid sequence of “GPRv21” against SWISS-PROT is shown in FIG. 4. “GPRv21” exhibited the highest homology (30%) to BOVIN NEUROPEPTIDE Y RECEPTOR TYPE 2 (P79113, 384 aa) of known G protein-coupled receptors. Thus, “GPRv21” was concluded to be a novel G protein-coupled receptor.

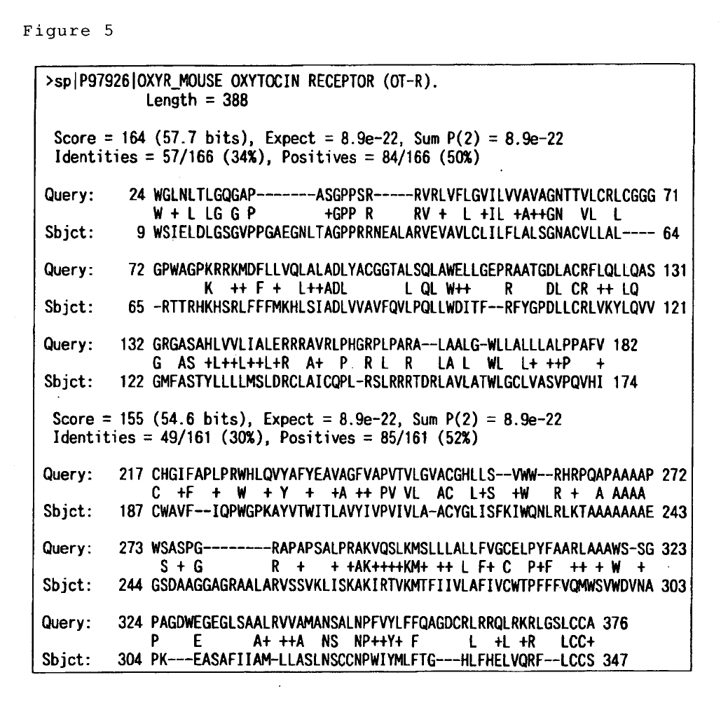

The result of BLAST SEARCH of the amino acid sequence of “GPRv40” against SWISS-PROT is shown in FIG. 5. “GPRv40” was not identical to any of known G protein-coupled receptors, but exhibited the highest homology (34%) to OXYTOCIN RECEPTOR (P97926, 388 aa). Thus, “GPRv40” was concluded to be a novel G protein-coupled receptor.

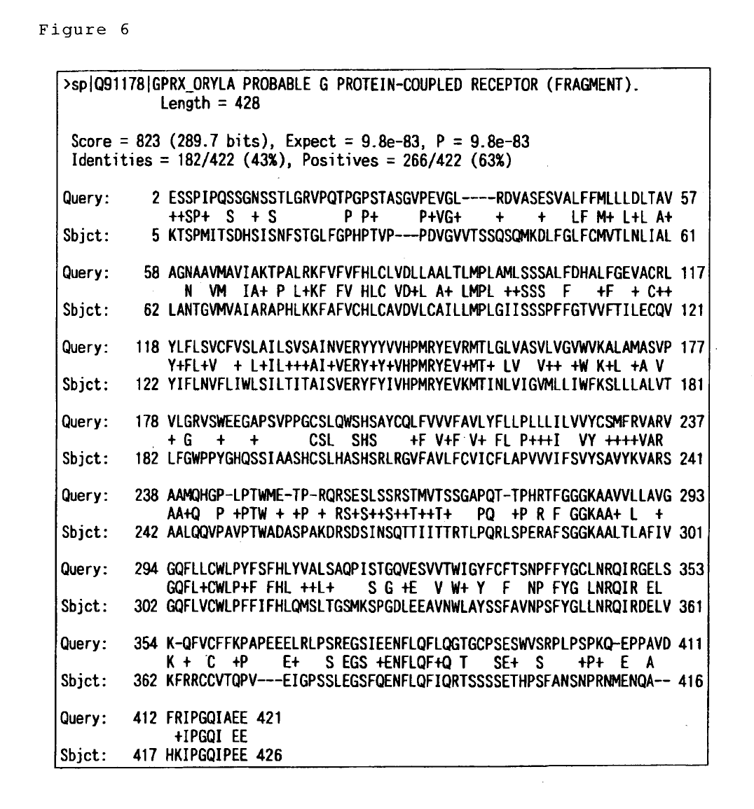

The result of BLAST SEARCH of the amino acid sequence of “GPRv47” against SWISS-PROT is shown in FIG. 6. “GPRv47” was not identical to any of known G protein-coupled receptors, but exhibited the highest homology (43%) to GPRX_ORYLA PROBABLE G PROTEIN-COUPLED RECEPTOR (Q91178, 428 aa). Thus, “GPRv47” was concluded to be a novel G protein-coupled receptor.

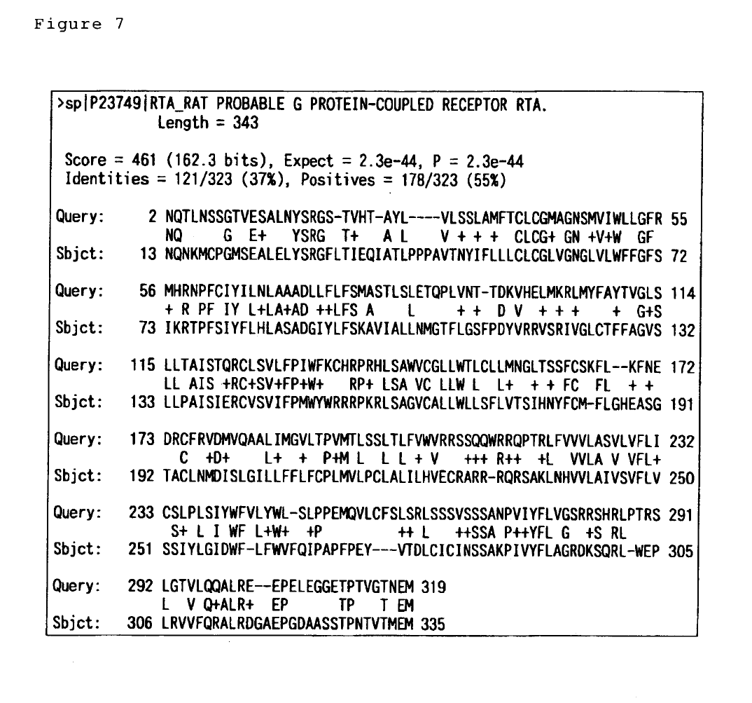

The result of BLAST SEARCH of the amino acid sequence of “GPRv51” against SWISS-PROT is shown in FIG. 7. “GPRv51” was not identical to any of known G protein-coupled receptors, but exhibited the highest homology (37%) to PROBABLE G PROTEIN-COUPLED RECEPTOR RTA (P23749, 343 aa). Thus, “GPRv51” was concluded to be a novel G protein-coupled receptor.

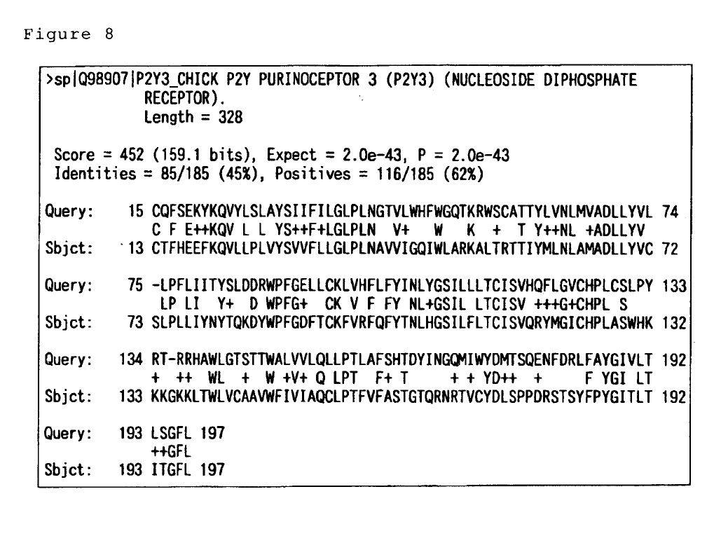

The result of BLAST SEARCH of the amino acid sequence of “GPRv71” against SWISS-PROT is shown in FIG. 8. “GPRv71” was not identical to any of known G protein-coupled receptors, but exhibited the highest homology (45%) to Chicken P2Y PURINOCEPTOR 3 (P2Y3) (Q98907, 328 aa). Thus, “GPRv71” was concluded to be a novel G protein-coupled receptor.

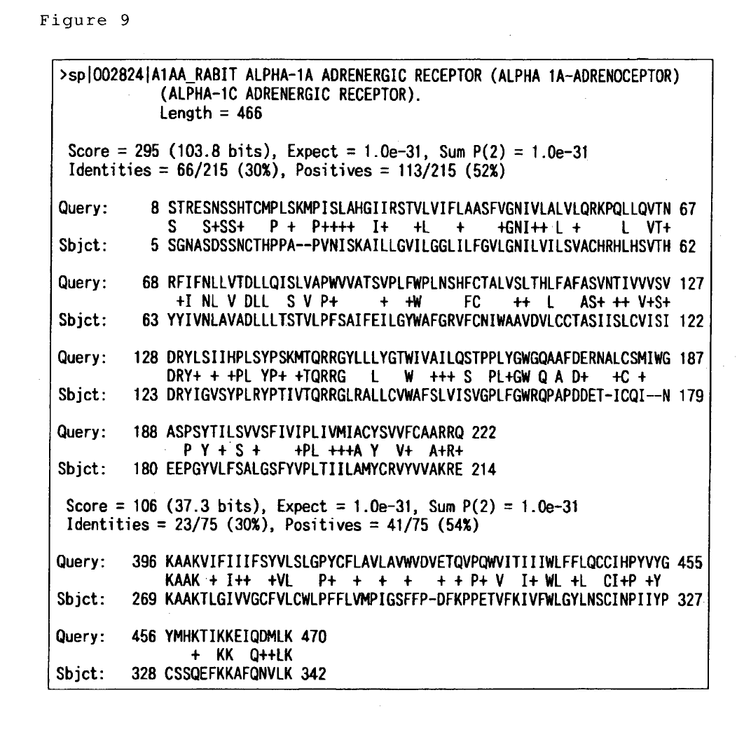

The result of BLAST SEARCH of the amino acid sequence of “GPRv72” against SWISS-PROT is shown in FIG. 9. “GPRv72” was not identical to any of known G protein-coupled receptors, but exhibited the highest homology (30%) to ALPHA-1A ADRENERGIC RECEPTOR (O02824, 466 aa). Thus, “GPRv72” was concluded to be a novel G protein-coupled receptor.

### EXAMPLE 3

### Analysis of Tissue-specific Expression

1. Reagents

1.1. Primers for Quantitative Polymerase Chain Reaction (PCR) and TaqMan Probes:

Sense primers, antisense primers,. and TaqMan probes were designed by using genetic analysis software “Primer Express version 1.0” from PE Biosystems. The ordinary custom-made primers and TaqMan probes were purchased from Amersham Pharmacia Biotech (Tokyo) and PE Biosystems Japan, respectively. The TaqMan probes were connected with a reporter pigment FAM at the 5′ end and with a quencher Tamra at the 3′ end. The nucleotide sequences of primers and TaqMan probes are shown below.

Synthetic DNA for GPRv8

| Synthetic DNA for GPRv8 | | | |
|---|---|---|---|
| PCR primer | G8.957F: | CCAGGAGCGTTTCTATGCCT | (SEQ ID NO: 37) |
| | G8.1082R: | TGTGATCTTTGCTCCCTGCA | (SEQ ID NO: 38) |
| TaqMan Probe | GPRv8.987T: | TCAGAACCTGCCAGCATTGAATAGTGCC | (SEQ ID NO: 39) |

Synthetic DNA for GPRv12

(SEQ ID NO: 40)

| | | |
|---|---|---|
| PCR primer | G12.794F: | ATCTGCTTTGCCCCGTATGT |

(SEQ ID NO: 41)

| | |
|---|---|
| G12.903R: | ACCGCCTTGCTGTAGGTCAG |

(SEQ ID NO: 42)

| | | |
|---|---|---|
| TaqMan Probe | GPRv12.834T: | TCGTGCCCTTCGTCACCGTGAA |

| Synthetic DNA for GPRv16 | | | |
|---|---|---|---|
| PCR primer | G16.1133F: | CCCAGCATCCATACCAGAAAA | (SEQ ID NO: 43) |
| | G16.1254R: | CTGTGTCCCTCTCATGCCAAA | (SEQ ID NO: 44) |
| TaqMan Probe | GPRv16.1193T: | TGAGAAGGCAGAGATTCCCATCCTTCCT | (SEQ ID NO: 45) |

| Synthetic DNA for GPRv21 | | | |
|---|---|---|---|
| PCR primer | G21.989F: | TCGCCATGAGCAACAGCAT | (SEQ ID NO: 46) |
| | G21.1114R: | CACTGGACTTACCGCCATTGT | (SEQ ID NO: 47) |
| TaqMan Probe | GPRv21.1064T: | AGATCATGTTGCTCCACTGGAAGGCTTCT | (SEQ ID NO: 48) |

Synthetic DNA for GPRv40

(SEQ ID NO: 49)

| | | |
|---|---|---|
| PCR primer | G40.16F: | GGATCTCTTTAGCCCCTCAATTC |

(SEQ ID NO: 50)

| | |
|---|---|
| G40.99R: | AAGGTCAGGTTGAGACCCCAG |

(SEQ ID NO: 51)

| | | |
|---|---|---|
| TaqMan Probe | GPRv40.53T: | AACATTTCCGTGCCCATCTTGCTGG |

| Synthetic DNA for GPRv47 | | | |
|---|---|---|---|
| PCR primer | G47.1292F: | GCTGTTGACTTTCGAATCCCA | (SEQ ID NO: 52) |
| | G47.1393R: | ACGGAGGTAGCTGTCTGACATGA | (SEQ ID NO: 53) |
| TaqMan Probe | GPRv47.1336T: | TGAGTTCCTGGAGCAGCAACTCACCA | (SEQ ID NO: 54) |

| Synthetic DNA for GPRv51 | | | |
|---|---|---|---|
| PCR primer | G51.190F: | GGCTTTCGAATGCACAGGAA | (SEQ ID NO: 55) |
| | G51.276R: | GGAAGCCATGCTGAAGAGGA | (SEQ ID NO: 56) |
| TaqMan Probe | GPRv51.214T: | TTCTGCATCTATATCCTCAACCTGGCGG | (SEQ ID NO: 57) |

| Synthetic DNA for GPRv71 | | | |
|---|---|---|---|
| PCR primer | G71.746F: | TGGCCTCTTCACCCTCTGTTT | (SEQ ID NO: 58) |
| | G71.841R: | ATCAAGAGCTGGCAGTCCTGA | (SEQ ID NO: 59) |
| TaqMan Probe | GPRv71.775T: | TCCATATCACTCGCTCCTTCTACCTCACCA | (SEQ ID NO: 60) |

| Synthetic DNA for GPRv72 | | | |
|---|---|---|---|
| PCR primer | G72.101F: | CCAAAATGCCCATCAGCCT | (SEQ ID NO: 61) |
| | G72.190R: | GCACTATGTTGCCGACGAAA | (SEQ ID NO: 62) |
| TaqMan Probe | GPRv72.132T: | CATCCGCTCAACCGTGCTGGTTATCT | (SEQ ID NO: 63) |

1.2. cDNA Derived from Patients

As cDNAs which had been derived from tumor and normal tissues from a single patient, Matched cDNA Pairs from Clontech were used. The tissues are lung, stomach, colon, ovary, prostate, uterus, and kidney.

Some cDNAs derived from following tissues were purchased from BioChain Institute: brain, pancreas, and testis from patients with tumor and normal adults; liver from cirrhosis patients and normal adults; kidney from lupus disease patients; and the hippocampus and frontal lobe from Alzheimer's disease (AD) patients and normal adults.

1.3. Reagents for Quantitative PCR:

TaqMan Universal PCR Master Mix (PE Biosystems) was used in this assay. TaqMan β-actin Control Reagents (PE Biosystems) was used for measuring the internal standard.

2. Quantitative PCR:

1) Dilution of Template cDNA

The cDNAs from BioChain were diluted 50 fold with water, and the cDNAs from Clontech were diluted 5 fold with water, for use.

2) Preparation of Master Mix

A reaction solution with the following composition was prepared.

| | Reaction volume | Preparation volume |
|---|---|---|
| 2x Master Mix | 12.5 μl | 1380 μl |
| Sense primer (50 μM) | 0.5 μl | 55.2 μl |
| Antisense primer (50 μM) | 0.5 μl | 55.2 μl |
| TaqMan Probe (5 μM) | 1 μl | 110.4 μl |
| Template cDNA | 2.5 μl | |
| Purified water | 8 μl | 883.2 μl |
| Total volume | 25 μl | 2484 μl |

3) Preparation of PCR Solution

6 μl template cDNA solution was added to 54 μl Master Mix solution. Then, 25-μl aliquots of the mixture were added in duplicate to the sample wells of a PCR plate to be placed in a device for quantitative PCR. A 25-μl aliquot of the above-mentioned Master Mix was added to each of two wells for non-template control. The standard curve was produced using eight 10-fold serial dilutions of cDNA which had been subcloned into pCEP4 vector, where the dilution started from 100 pg/μl. A 25-μl aliquot of each mixture obtained by combining 54 μl of Master Mix prepared in Section 2) and 6 μl of each standard solution prepared above was added into a standard well. Namely, the largest amount of the plasmid DNA was 250 pg and the smallest was 25 ag (a: atto, 10⁻¹⁸) in the standard wells. After 8-cap strips were placed to the top of the wells, the bubbles were removed by light centrifugation.

4) PCR

The plate was placed in the device for quantitative PCR (GeneAmp 5700 Sequence Detection System: PE Biosystems), and then the reaction was carried out according to the following cycling program.

| | | |
|---|---|---|
| (1) 50° C., 2 minutes: | | 1 cycle |
| (2) 95° C., 10 minutes | | 1 cycle |
| (3) 95° C., 15 seconds | | 50 cycles |
| | {close oversize brace} | |
| 60° C., 1 minutes | | |

5) Quantitative Analysis

The quantification was carried out according to the operation manual of GeneAmp 5700, and the result was outputted.

3. Results and Conclusions:

The GPCR expression profiles obtained with the cDNAs from the organs from normal human and those from patients with disease were represented as ratios relative to the expression level of the actin gene as an internal standard. The experiment was carried out in duplicate, and the average values are shown in Table 1.

TABLE 1

| | relative copy number | | | | | | | | |
|---|---|---|---|---|---|---|---|---|---|
| | GPRv8 | GPRv12 | GPRv16 | GPRv21 | GPRv40 | GPRv47 | GPRv51 | GPRv71 | GPRv72 |
| Brain Normal¹⁾ | 0 | 0 | 1 | 0 | 6 | 9 | 0 | 0 | 0 |
| Tumor¹⁾ | 5 | 2 | 11 | 0 | 23 | 76 | 2 | 5 | 0 |
| Lung Normal | 0 | 0 | 1 | 0 | 11 | 0 | 1 | 1 | 0 |
| Tumor | 1 | 0 | 1 | 0 | 11 | 2 | 1 | 1 | 1 |
| Stomach Normal | 6 | 0 | 0 | 0 | 29 | 0 | 1 | 1 | 0 |
| Tumor | 3 | 0 | 2 | 0 | 1 | 0 | 3 | 0 | 1 |
| Pancreas Normal¹⁾ | 0 | 0 | 0 | 0 | 4 | 0 | 0 | 0 | 0 |
| Tumor¹⁾ | 45 | 2 | 0 | 0 | 23 | 2 | 3 | 4 | 1 |
| Colon Normal | 141 | 0 | 61 | 11 | 119 | 50 | 111 | 44 | 113 |
| Tumor | 2766 | 0 | 0 | 0 | 110 | 21 | 6 | 2 | 0 |
| Ovary Normal | 0 | 0 | 1 | 0 | 2 | 1 | 2 | 1 | 1 |
| Tumor | 0 | 4 | 0 | 0 | 21 | 1 | 3 | 3 | 0 |
| Uterus Normal | 0 | 0 | 3 | 0 | 7 | 0 | 3 | 3 | 1 |
| Tumor | 19 | 0 | 0 | 0 | 9 | 1 | 21 | 8 | 1 |
| Prostate Normal | 0 | 0 | 0 | 0 | 18 | 1 | 3 | 1 | 0 |
| Tumor | 6 | 0 | 0 | 0 | 9 | 0 | 8 | 3 | 0 |
| Testis Normal¹⁾ | 18 | 0 | 10 | 5 | 3 | 22 | 20 | 2 | 1 |
| Tumor¹⁾ | 8 | 3 | 13 | 0 | 21 | 3 | 3 | 2 | 0 |
| Kidney Normal | 9 | 0 | 0 | 0 | 29 | 0 | 27 | 3 | 5 |
| Tumor | 9 | 0 | 0 | 0 | 28 | 10 | 15 | 0 | 0 |
| Lupus¹⁾ | 25 | 0 | 1 | 0 | 1 | 0 | 3 | 1 | 0 |
| Liver Normal¹⁾ | 0 | 0 | 10 | 0 | 27 | 11 | 13 | 5 | 1 |
| Cirrhosis¹⁾ | 1 | 0 | 0 | 0 | 4 | 0 | 2 | 0 | 0 |
| Hippocampus Normal¹⁾ | 6 | 12 | 4 | 0 | 40 | 113 | 2 | 5 | 2 |
| AD¹⁾ | 16 | 1 | 50 | 3 | 111 | 63 | 55 | 12 | 27 |
| Frontal lobe Normal¹⁾ | 3 | 2 | 8 | 0 | 16 | 140 | 3 | 8 | 1 |
| AD¹⁾ | 2 | 1 | 1 | 0 | 9 | 29 | 2 | 2 | 0 |

When a 3-fold or more alternation in the expression level was reproducible, the difference is assessed as being significant. The cDNAs derived from the organs marked with ¹⁾ were purchased from BioChain; and the cDNAs derived from the organs without the mark were purchased from Clontech. The disease-dependent differences in the expression levels of the respective genes are summarized below.

The expression of GPRv8 was undetectable in the normal pancreas and uterus, but GPRv8 was expressed at a moderate level after canceration. GPRv8 was strongly expressed in the colon, and was more strongly expressed in colon cancer.

The expression level of GPRv12 was generally low. The expression was undetectable in the normal ovary and testis, but was found after canceration. The expression level decreased in the hippocampus with Alzheimer's disease.

GPRv16 was expressed in the colon, but was undetectable after canceration. The expression level increased in the brain after canceration. In the liver, the expression was undetectable after cirrhosis. In the brain of Alzheimer's disease patients, the expression level was elevated in the hippocampus.

The expression level of GPRv21 was low, and was undetectable in the colon and testis after canceration.

The expression level of GPRv40 increased in the brain and testis after canceration, and decreased in the liver after cirrhosis.

The expression level of GPRv47 increased in the brain and kidney and decreased in the testis after canceration. The expression was undetectable in the liver after cirrhosis.

GPRv51 was strongly expressed in the colon, but the expression level decreased after canceration. The expression level decreased in the testis after canceration. The expression level also decreased in the liver after cirrhosis as compared to the normal liver. The expression level was low in the brain, but increased in the hippocampus with Alzheimer's disease.

The expression level of GPRv71 decreased in the colon and kidney after canceration, and the expression thereof was undetectable in the liver after cirrhosis. In the patient with Alzheimer's disease, the expression level decreased in the frontal lobe.

GPRv72 was expressed strongly in the colon, but the expression thereof was undetectable after canceration. The expression level was low in the brain, but increased in the hippocampus with Alzheimer's disease.

### EXAMPLE 4

### Analysis of GPRv8 with Bioinformatics

1. Homology Search of GPRv8

The amino acid sequence of GPRv8 was analyzed by searching known sequences (the known sequence databases are produced in EMBL (Release 64, http://www.ebi.ac.uk/), GENBANK (Release 120.0, http://www.ncbi.nlm.nih.gov/), and PIR (Release 66.00, http://www-nbrf.georgetown.edu/pir/)) with an analysis program (BLAST 2.0) (Altschul, Stephen F. et al. (1997) Nucleic Acids Res. 25:3389-3402.). The result showed that GPRv8 had homology to the sequences shown in Table 2. Thus, GPRv8 was revealed to be a novel clone having homology to GPCR. The amino acid sequence of GPRv8 was analyzed by searching known sequences with an analysis program (BLAST 2.0); the result (data with the E-value lower than e-39) is shown in Table 2.

TABLE 2

| Hit | E- | | |
|---|---|---|---|
| (ID) | value | Identities % | Description |
| AE003754 | 2e−68 | 43 | gene: “CG6111”- |
| | | | Drosophila melanogaster |
| AF147743 | 7e−43 | 33 | vasotocin VT1 receptor- |
| | | | Gallus gallus |
| AF184966 | 2e−42 | 33 | arginine vasotocin receptor- |
| | | | Platichthys flesus |
| X93313 | 4e−42 | 36 | mesotocin receptor- |
| | | | giant toad |
| X76321 | 8e−42 | 32 | vasotocin receptor- |
| | | | white sucker |
| X87783 | 4e−41 | 33 | isotocin receptor- |
| | | | white sucker |
| X64878 | 3e−40 | 32 | oxytocin receptor- H. sapiens |
| U82440 | 7e−40 | 32 | oxytocin receptor- |
| | | | Macaca mulatta |

2. Prediction of Transmembrane Domain

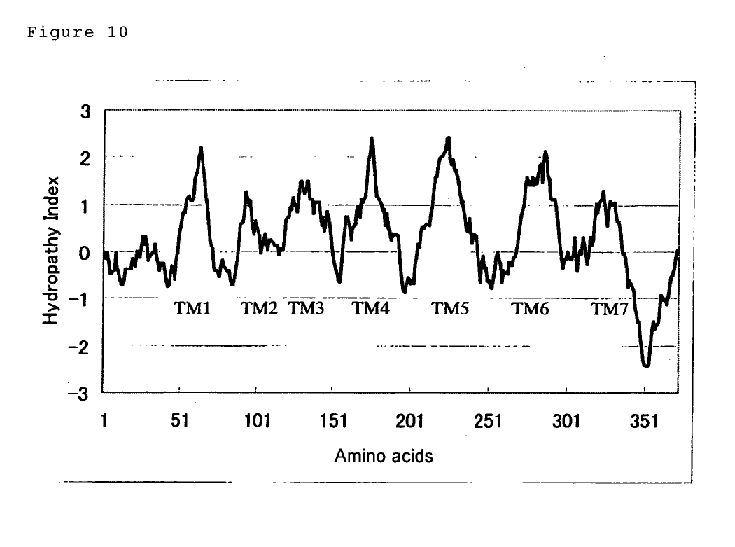

The amino acid sequence of GPRv8 was analyzed according to the method of Kyte-Doolittle (J. Kyte and R. F. Doolittle, (1982), J. Mol. Biol., 157,105-132.), for obtaining a hydropathy plot and used to predict the transmembrane domain. The result showed that GPRv8 had seven transmembrane domains (TM1-TM7) (FIG. 10).

3. HMMPfam Search

Using the amino acid sequence of GPRv8 as the query, PFAM search based on the hidden Markov model (HMMPFAM (Sonnhammer E L, et al., Nucleic Acids Res Jan. 1, 1998; 26 (1):320-322)) was carried out. The search was carried out with the hidden Markov model of HMMER version 2.1 (http://hmmer.wustl.edu/) and the PFAM database of Pfam Version 5.5 (http://www.sanger.ac.uk/Software/Pfam/index.shtml).

The result indicated that GPRv8 comprises tm7_{—}1 (Rhodopsin family). The result of HMMPfam search is shown in Table 3.

TABLE 3

| | | | Q | Q | |
|---|---|---|---|---|---|
| Hit | Score | Expect | from | to | Description |
| 7tm_1 | 164.2 | 5.1e−51 | 66 | 330 | 7 transmembrane |
| | | | | | receptor |
| | | | | | (rhodopsin family) |

Hit: name of the domain deduced by the search.
Score: the higher the value, the higher the reliability.
Expect: as the value approaches 0, the reliability becomes higher.
Q from: the start position of the deduced domain.
Q to: the termination position of the deduced domain.
Description: explanation of the deduced domain.

4. Amino Acid Sequence Alignment

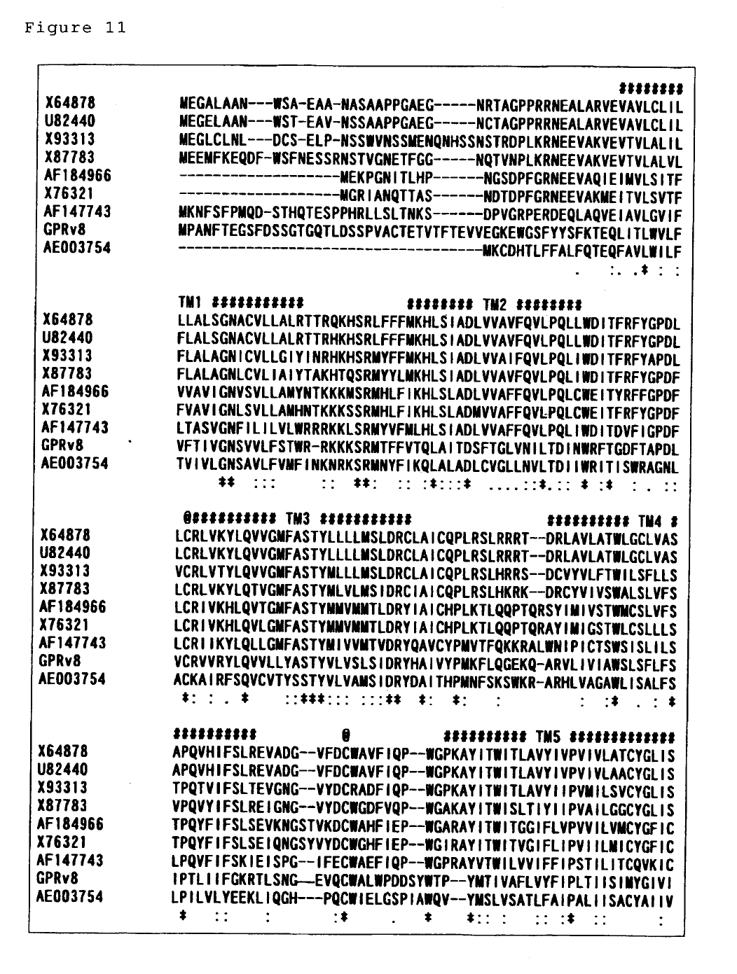

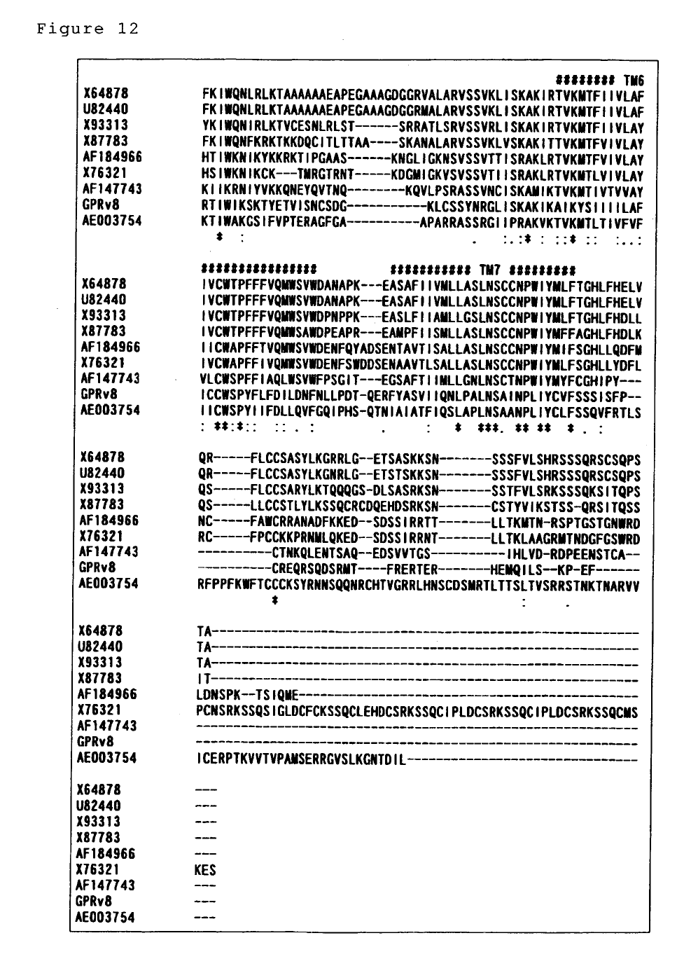

The amino acid sequences of GPRv8 and proteins shown in Table 2 were aligned together by using Clustalw 1.7 (FIGS. 11 and 12). The result showed that GPRv8 comprise seven transmembrane domains (### ###) and Cys (Cys marked with “@”) participating in specific S—S bonding of GPCR.

### EXAMPLE 5

### Analysis of GPRv12 with Bioinformatics

1. Homology Search of GPRv12

The amino acid sequence of GPRv12 was analyzed by searching known sequences (the known sequence databases are produced in EMBL (Release 64, http://www.ebi.ac.uk/), GENBANK (Release 120.0, http://www.ncbi.nlm.nih.gov/), and PIR (Release 66.00, http://www-nbrf.georgetown.edu/pir/)) with an analysis program (BLAST 2.0) (Altschul, Stephen F. et al. (1997) Nucleic Acids Res. 25:3389-3402.). The result showed that GPRv12 had homology to the sequences shown in Table 4. Thus, GPRv12 was revealed to be a novel clone having homology to GPCR. The amino acid sequence of GPRv12 was analyzed by searching known sequences with an analysis program (BLAST 2.0); the result (data with the E-value lower than e-15) is shown in Table 4.

TABLE 4

| Hit | E- | | |
|---|---|---|---|
| (ID) | value | Identities % | Description |
| AF208288 | 8e−88 | 50 | orphan G protein-coupled |
| | | | receptor GPR26- |
| | | | Rattus norvegicus |
| L03202 | 2e−17 | 24 | 5-hydroxytryptamine receptor- |
| | | | rat |
| L41146 | 5e−17 | 23 | 5-HT6 serotonin receptor- |
| | | | Rattus norvegicus |
| S62043 | 2e−16 | 25 | serotonin receptor 6-rat |
| L41147 | 2e−16 | 24 | 5-HT6 serotonin receptor- |
| | | | Homo sapiens |
| AF134158 | 4e−16 | 23 | serotonin 6 receptor- |
| | | | Mus musculus |
| L14856 | 4e−16 | 26 | somatostatin receptor 4- Human |
| Y14627 | 5e−16 | 21 | Dopamine receptor- |
| | | | Cyprinus carpio |
| L07833 | 6e−16 | 26 | somatostatin receptor 4- |
| | | | Homo sapiens |
| AF069547 | 8e−16 | 21 | putative odorant receptor LOR4- |
| | | | Lampetra fluviatilis |

2. Prediction of Transmembrane Domain

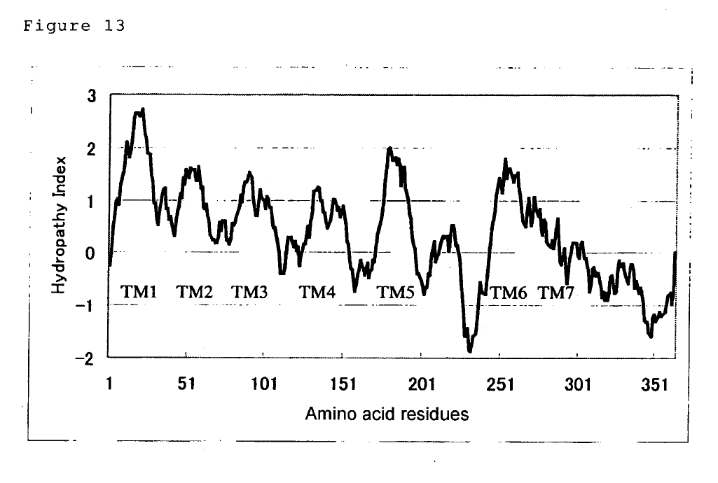

The amino acid sequence of GPRv12 was analyzed according to the method of Kyte-Doolittle (J. Kyte and R. F. Doolittle, (1982), J. Mol. Biol., 157,105-132.), for obtaining a hydropathy plot and used to predict the transmembrane domain. The result showed that GPRv12 had seven transmembrane domains (TM1-TM7) (FIG. 13).

3. HMMPfam Search

Using the amino acid sequence of GPRv12 as the query, PFAM search based on the hidden Markov model (HMMPFAM (Sonnhammer E L, et al., Nucleic Acids Res Jan. 1, 1998; 26 (1) :320-322)) was carried out. The search was carried out with the hidden Markov model of HMMER version 2.1 (http://hmmer.wustl.edu/) and the PFAM database of Pfam Version 5.5 (http://www.sanger.ac.uk/Software/Pfam/index.shtml).

The result indicated that GPRv12 comprises tm7_{—}1 (Rhodopsin family). The result of HMMPfam search is shown in Table 5.

TABLE 5

| Hit | Score | Expect | Q from | Q to | Description |
|---|---|---|---|---|---|
| 7tm_1 | 74.7 | 7.7e−23 | 22 | 294 | 7 transmembrane |
| | | | | | receptor |
| | | | | | (rhodopsin family) |

Hit: name of the domain deduced by the search.
Score: the higher the value, the higher the reliability.
Expect: as the value approaches 0, the reliability becomes higher.
Q from: the start position of the deduced domain.
Q to: the termination position of the deduced domain.
Description: explanation of the deduced domain.

4. Amino Acid Sequence Alignment

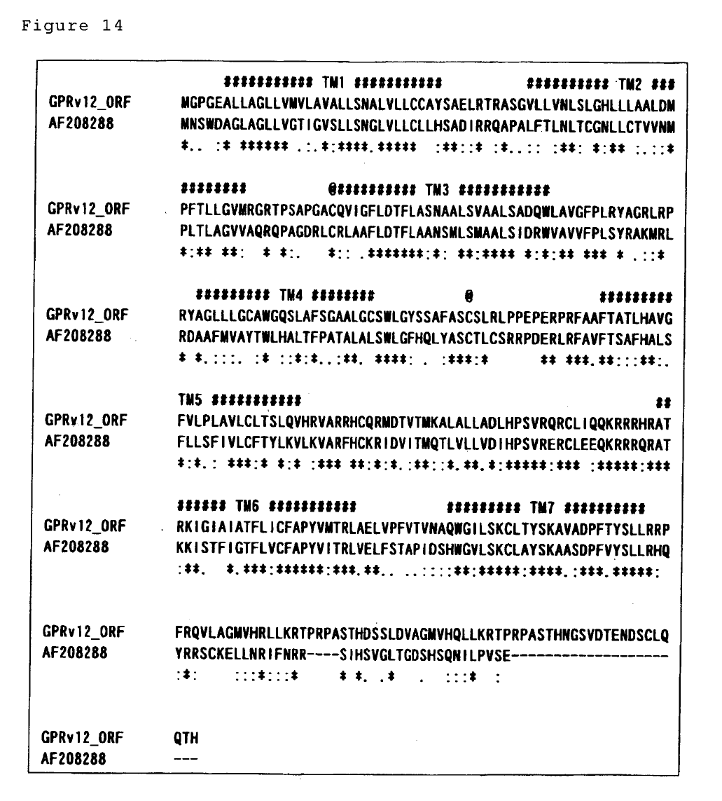

The amino acid sequences of GPRv12 and orphan G protein-coupled receptor GPR26—Rattus norvegicus (AF208288) were aligned together by using Clustalw 1.7 (FIG. 14). The result showed that GPRv12 comprise seven transmembrane domains (### ###) and Cys (Cys marked with “@”) participating in specific S—S bonding of GPCR.

### EXAMPLE 6

### Analysis of GPRv16 with Bioinformatics

1. Homology Search of GPRv16

The amino acid sequence of GPRv16 was analyzed by searching known sequences (the known sequence databases are produced in EMBL (Release 64, http://www.ebi.ac.uk/), GENBANK (Release 120.0, http://www.ncbi.nlm.nih.gov/), and PIR (Release 66.00, http://www-nbrf.georgetown.edu/pir/)) with an analysis program (BLAST 2.0) (Altschul, Stephen F. et al. (1997) Nucleic Acids Res. 25:3389-3402.) The result showed that GPRv16 had homology to the sequences shown in Table 6. Thus, GPRv16 was revealed to be a novel clone having homology to GPCR. The amino acid sequence of GPRv16 was analyzed by searching known sequences with an analysis program (BLAST 2.0); the result (data with the E-value lower than e-18) is shown in Table 6.

TABLE 6

| Hit | E- | | |
|---|---|---|---|
| (ID) | value | Identities % | Description |
| AF042784 | 4e−20 | 23 | GALANIN RECEPTOR TYPE 2- |
| | | | Mus musculus |
| U30290 | 4e−20 | 27 | galanin receptor GALR1- |
| | | | Rattus norvegicus |
| U90657 | 6e−20 | 27 | GALANIN RECEPTOR TYPE 1- |
| | | | mouse |
| AF042782 | 7e−20 | 25 | galanin receptor type 2- |
| | | | Homo sapiens |
| U94322 | 1e−19 | 24 | galanin receptor type 2- |
| | | | Rattus norvegicus |
| AF077375 | 6e−19 | 23 | galanin receptor type 2- |
| | | | Mus musculus |

2. Prediction of Transmembrane Domain

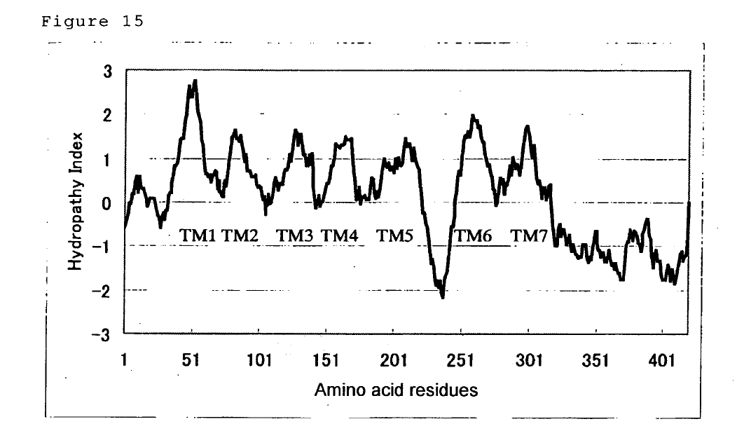

The amino acid sequence of GPRv16 was analyzed according to the method of Kyte-Doolittle (J. Kyte and R. F. Doolittle, (1982), J. Mol. Biol., 157,105-132.), for obtaining a hydropathy plot and used to predict the transmembrane domain. The result showed that GPRv16 had seven transmembrane domains (TM1-TM7) (FIG. 15).

3. HMMPfam Search

Using the amino acid sequence of GPRv16 as the query, PFAM search based on the hidden Markov model (HMMPFAM (Sonnhammer E L, et al., Nucleic Acids Res Jan. 1, 1998; 26 (1):320-322)) was carried out. The search was carried out with the hidden Markov model of HMMER version 2.1 (http://hmmer.wustl.edu/) and the PFAM database of Pfam Version 5.5 (http://www.sanger.ac.uk/Software/Pfam/index.shtml).

The result indicated that GPRv16 comprises tm7_{—}1 (Rhodopsin family). The result of HMMPfam search is shown in Table 7.

TABLE 7

| Hit | Score | Expect | Q from | Q to | Description |
|---|---|---|---|---|---|
| 7tm_1 | 23.8 | 8.3e−7 | 155 | 306 | 7 transmembrane |
| | | | | | receptor |
| | | | | | (rhodopsin family) |
| 7tm_1 | 13.3 | 0.0017 | 53 | 133 | 7 transmembrane |
| | | | | | receptor |
| | | | | | (rhodopsin family) |

Hit: name of the domain deduced by the search.
Score: the higher the value, the higher the reliability.
Expect: as the value approaches 0, the reliability becomes higher.
Q from: the start position of the deduced domain.
Q to: the termination position of the deduced domain.
Description: explanation of the deduced domain.

4. Amino Acid Sequence Alignment

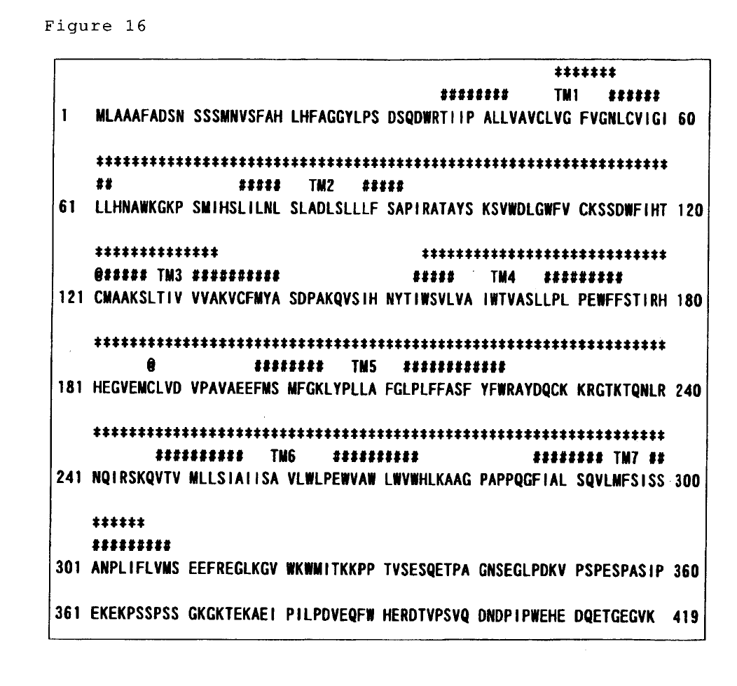

The result of sections 3 and 4 are indicated in FIG. 16. The result showed that GPRv16 comprise Cys (@) participating in specific S—S bonding of GPCR.

### EXAMPLE 7

### Analysis of GPRv21 with Bioinformatics

1. Homology Search of GPRv21

The amino acid sequence of GPRv21 was analyzed by searching known sequences (the known sequence databases are produced in EMBL (Release 64, http://www.ebi.ac.uk/), GENBANK (Release 120.0, http://www.ncbi.nlm.nih.gov/), and, PIR (Release 66.00, http://www-nbrf.georgetown.edu/pir/)) with an analysis program (BLAST 2.0) (Altschul, Stephen F. et al. (1997) Nucleic Acids Res. 25:3389-3402.) The result showed that GPRv21 had homology to the sequences shown in Table 8. Thus, GPRv21 was revealed to be a novel clone having homology to GPCR. The amino acid sequence of GPRv21 was analyzed by searching known sequences with an analysis program (BLAST 2.0); the result (data with the E-value lower than e-35) is shown in Table 8.

TABLE 8

| Hit | E- | | |
|---|---|---|---|
| (ID) | value | Identities % | Description |
| AL121755 | 0.0 | 89 | G-protein coupled receptor- |
| | | | Human |
| AF236082 | 0.0 | 83 | G-protein coupled |
| | | | receptor GPR73- |
| | | | Mus musculus |
| M81490 | 9e−37 | 34 | neuropeptide receptor- |
| | | | D. melanogaster |
| U50144 | 3e−36 | 30 | type 2 neuropeptide Y receptor- |
| | | | Bos taurus |
| U42766 | 6e−36 | 29 | neuropeptide y2 receptor- Human |
| AF037444 | 8e−36 | 28 | cardioexcitatory receptor- |
| | | | Lymnaea stagnalis |
| D86238 | 8e−36 | 28 | neuropeptideY-Y2 receptor- |
| | | | Mus musculus |
| U42389 | 8e−36 | 29 | neuropeptide y/peptide YY |
| | | | receptor type 2- |
| | | | human |
| U76254 | 8e−36 | 29 | neuropeptide Y receptor type 2- |
| | | | Human |

2. Prediction of Transmembrane Domain

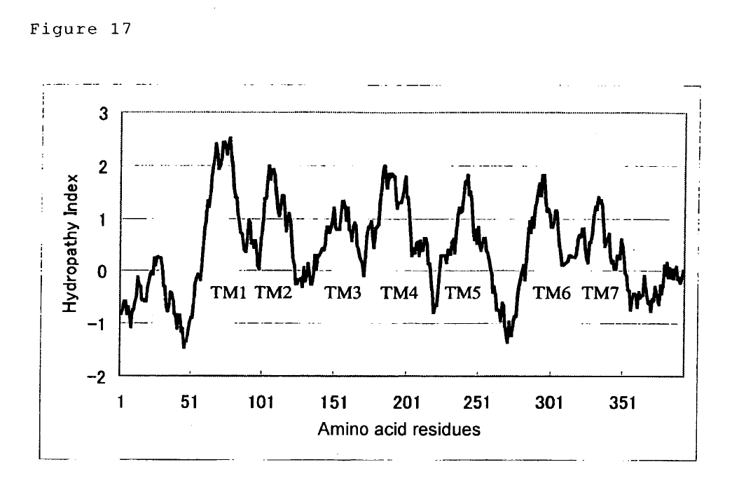

The amino acid sequence of GPRv21 was analyzed according to the method of Kyte-Doolittle (J. Kyte and R. F. Doolittle, (1982), J. Mol. Biol. , 157,105-132.), for obtaining a hydropathy plot and used to predict the transmembrane domain. The result showed that GPRv21 had seven transmembrane domains (TM1-TM7) (FIG. 17).

3. HMMPfam Search

Using the amino acid sequence of GPRv21 as the query, PFAM search based on the hidden Markov model (HMMPFAM (Sonnhammer E L, et al., Nucleic Acids Res Jan. 1, 1998; 26 (1):320-322)) was carried out. The search was carried out with the hidden Markov model of HMMER version 2.1 (http://hmmer.wustl.edu/) and the PFAM database of Pfam Version 5.5 (http://www.sanger.ac.uk/Software/Pfam/index.shtml).

The result indicated that GPRv21 comprises tm7_{—}1 (Rhodopsin family). The result of HMMPfam search is shown in Table 9.

TABLE 9

| Hit | Score | Expect | Q from | Q to | Description |
|---|---|---|---|---|---|
| 7tm_1 | 188.1 | 1.6e−58 | 79 | 338 | 7 transmembrane |
| | | | | | receptor |
| | | | | | (rhodopsin family) |

Hit: name of the domain deduced by the search.
Score: the higher the value, the higher the reliability.
Expect: as the value approaches 0, the reliability becomes higher.
Q from: the start position of the deduced domain.
Q to: the termination position of the deduced domain.
Description: explanation of the deduced domain.

4. Amino Acid Sequence Alignment

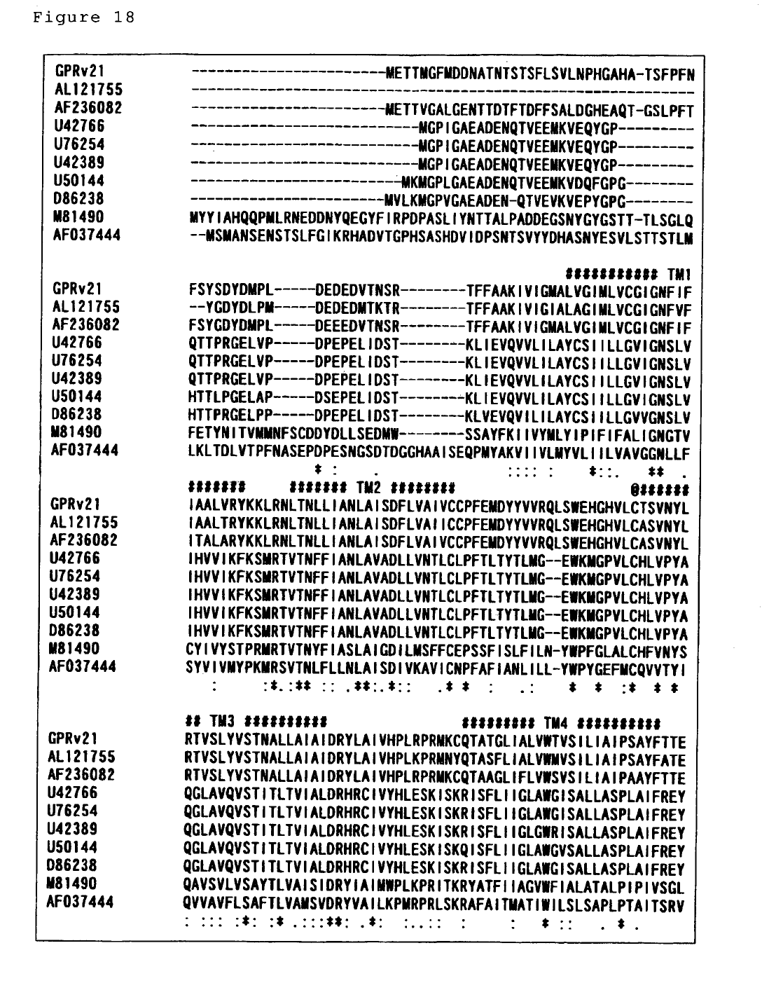

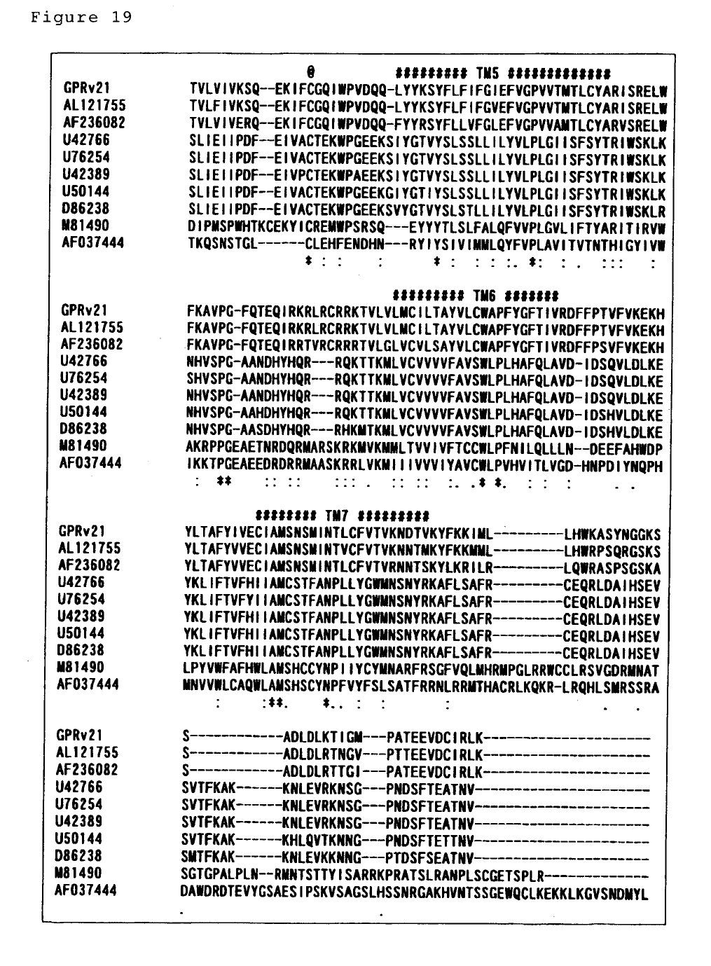

The amino acid sequences of GPRv21 and proteins shown in Table 8 were aligned together by using Clustalw 1.7 (FIGS. 18 and 19). The result showed that GPRv21 comprise seven transmembrane domains (### ###) and Cys (Cys marked with “@”) participating in specific S—S bonding of GPCR.

### EXAMPLE 8

### Analysis of GPRv40 with Bioinformatics

1. Homology Search of GPRv40

The amino acid sequence of GPRv40 was analyzed by searching known sequences (the known sequence databases are produced in EMBL (Release 64, http://www.ebi.ac.uk/), GENBANK (Release 120.0, http://www.ncbi.nlm.nih.gov/), and PIR (Release 66.00, http://www-nbrf.georgetown.edu/pir/)) with an analysis program (BLAST 2.0) (Altschul, Stephen F. et al. (1997) Nucleic Acids Res. 25:3389-3402.). The result showed that GPRv40 had homology to the sequences shown in Table 10. Thus, GPRv40 was revealed to be a novel clone having homology to GPCR. The amino acid sequence of GPRv40 was analyzed by searching known sequences with an analysis program (BLAST 2.0); the result (data with the E-value lower than e-11) is shown in Table 10.

TABLE 10

| Hit | E- | | |
|---|---|---|---|
| (ID) | value | Identities % | Description |
| D86599 | 1e−13 | 23 | oxytocin receptor- Mus sp. |
| U15280 | 4e−13 | 23 | oxytocin receptor- |
| | | | Rattus norvegicus |
| X76321 | 1e−12 | 22 | vasotocin receptor- |
| | | | white sucker |
| X64878 | 2e−12 | 21 | oxytocin receptor- |
| | | | H. sapiens |
| X87783 | 2e−12 | 21 | isotocin receptor- |
| | | | C. commersoni |
| D45400 | 3e−12 | 23 | vasopressin receptor V1b- |
| | | | rat |
| L37112 | 3e−12 | 24 | vasopressin receptor |
| | | | subtype 1b- |
| | | | Homo sapiens |
| U27322 | 6e−12 | 23 | arginine-vasopressin V1b |
| | | | receptor- |
| | | | Rattus norvegicus |
| U82440 | 6e−12 | 21 | oxytocin receptor- |
| | | | Macaca mulatta |

2. Prediction of Transmembrane Domain

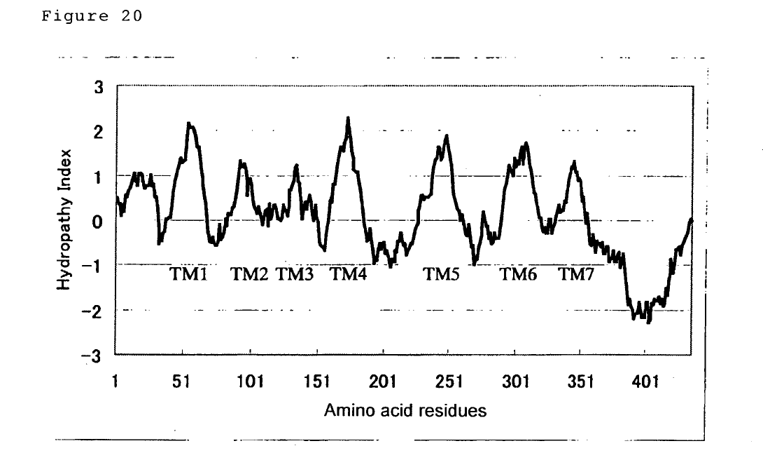

The amino acid sequence of GPRv40 was analyzed according to the method of Kyte-Doolittle (J. Kyte and R. F. Doolittle, (1982), J. Mol. Biol., 157,105-132.), for obtaining a hydropathy plot and used to predict the transmembrane domain. The result showed that GPRv40 had seven transmembrane domains (TM1-TM7) (FIG. 20).

3. HMMPfam Search

Using the amino acid sequence of GPRv40 as the query, PFAM search based on the hidden Markov model (HMMPFAM (Sonnhammer E L, et al. , Nucleic Acids Res Jan. 1, 1998; 26 (1):320-322)) was carried out. The search was carried out with the hidden Markov model of HMMER version 2.1 (http://hmmer.wustl.edu/) and the PFAM database of Pfam Version 5.5 (http://www.sanger.ac.uk/Software/Pfam/index.shtml).

The result indicated that GPRv40 comprises tm7_{—}1 (Rhodopsin family). The result of HMMPfam search is shown in Table 11.

TABLE 11

| | | | Q | Q | |
|---|---|---|---|---|---|
| Hit | Score | Expect | from | to | Description |
| 7tm_1 | 26.5 | 1.1e−07 | 228 | 352 | 7 transmembrane |
| | | | | | receptor |
| | | | | | (rhodopsin family) |
| 7tm_1 | 18.1 | 5e−05 | 59 | 181 | 7 transmembrane |
| | | | | | receptor |
| | | | | | (rhodopsin family) |

Hit: name of the domain deduced by the search.
Score: the higher the value, the higher the reliability.
Expect: as the value approaches 0, the reliability becomes higher.
Q from: the start position of the deduced domain.
Q to: the termination position of the deduced domain.
Description: explanation of the deduced domain.

4. Amino Acid Sequence Alignment

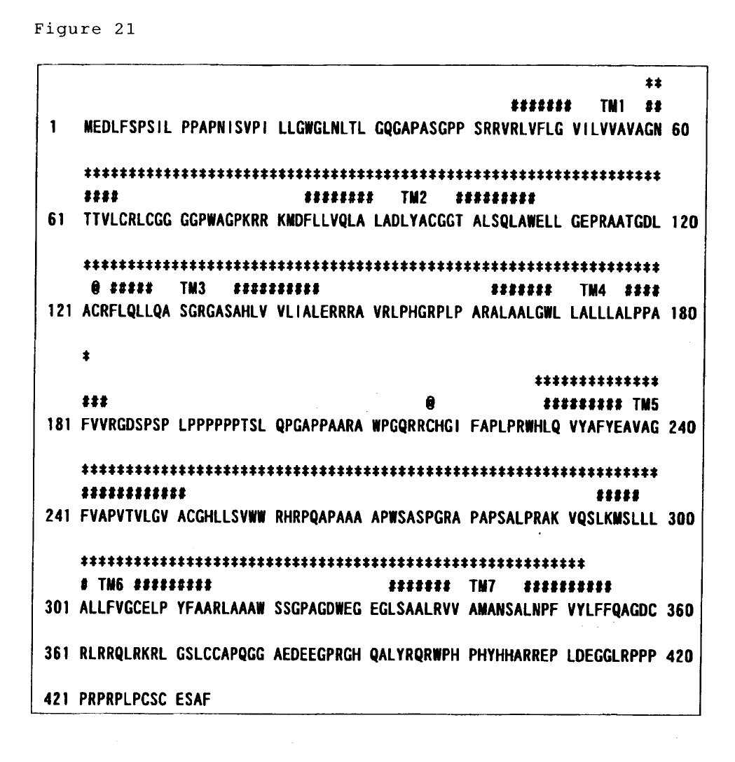

The result of section 3 and 4 are indicated in FIG. 21. The result showed that GPRv40 comprise Cys (@) participating in specific S—S bonding of GPCR.

### EXAMPLE 9

### Analysis of GPRv47 with Bioinformatics

1. Homology Search of GPRv47

The amino acid sequence of GPRv47 was analyzed by searching known sequences (the known sequence databases are produced in EMBL (Release 64, http://www.ebi.ac.uk/), GENBANK (Release 120.0, http://www.ncbi.nlm.nih.gov/), and PIR (Release 66.00, http://www-nbrf.georgetown.edu/pir/)) with an analysis program (BLAST 2.0) (Altschul, Stephen F. et al. (1997) Nucleic Acids Res. 25:3389-3402.). The result showed that GPRv47 had homology to the sequences shown in Table 12. Thus, GPRv47 was revealed to be a novel clone having homology to GPCR. The amino acid sequence of GPRv47 was analyzed by searching known sequences with an analysis program (BLAST 2.0); the result (data with the E-value lower than e-11) is shown in Table 12.

TABLE 12

| Hit | E- | | |
|---|---|---|---|
| (ID) | value | Identities % | Description |
| D43633 | 1e−85 | 41 | G protein-coupled |
| | | | 7-transmembrane receptor- |
| | | | Medaka fish |
| X98133 | 2e−28 | 27 | histamine H2 receptor- |
| | | | H. sapiens |
| M32701 | 3e−28 | 28 | histamine H2 receptor- |
| | | | Canine histamine |
| L41147 | 6e−28 | 31 | 5-HT6 serotonin receptor- |
| | | | Homo sapiens |
| U25440 | 8e−28 | 26 | histamine H2 receptor- |
| | | | Cavia porcellus |
| D49783 | 1e−27 | 28 | histamine H2 receptor- Human |
| U64032 | 2e−27 | 27 | alpha 1d adrenoceptor- |
| | | | Oryctolagus cuniculus |
| S73473 | 3e−27 | 28 | beta 3-adrenergic receptor- |
| | | | rats |
| M74716 | 4e−27 | 28 | beta-adrenergic receptor- Rat |
| S57565 | 6e−27 | 27 | histamine H2-receptor- rats |

2. Prediction of Transmembrane Domain

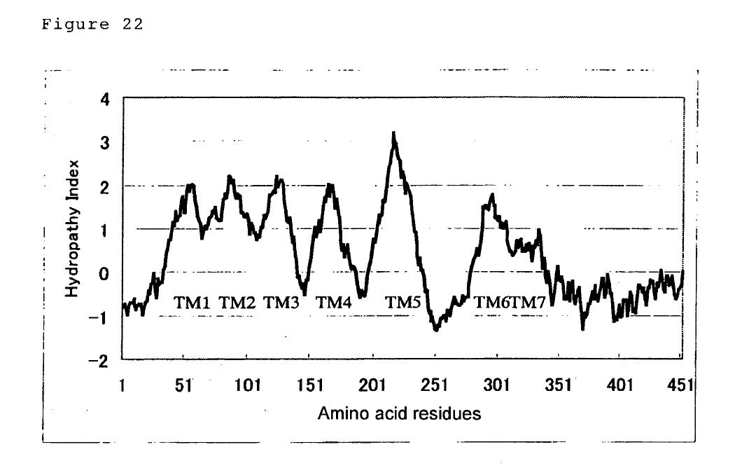

The amino acid sequence of GPRv47 was analyzed according to the method of Kyte-Doolittle (J. Kyte and R. F. Doolittle, (1982), J. Mol. Biol., 157,105-132.), for obtaining a hydropathy plot and used to predict the transmembrane domain. The result showed that GPRv47 had seven transmembrane domains (TM1-TM7) (FIG. 22).

3. HMMPfam Search

Using the amino acid sequence of GPRv47 as the query, PFAM search based on the hidden Markov model (HMMPFAM (Sonnhammer E L, et al. , Nucleic Acids Res Jan. 1, 1998; 26 (1):320-322)) was carried out. The search was carried out with the hidden Markov model of HMMER version 2.1 (http://hmmer.wustl.edu/) and the PFAM database of Pfam Version 5.5 (http://www.sanger.ac.uk/Software/Pfam/index.shtml).

The result indicated that GPRv47 comprises tm7_{—}1 (Rhodopsin family). The result of HMMPfam search is shown in Table 13.

TABLE 13

| Hit | Score | Expect | Q from | Q to | Description |
|---|---|---|---|---|---|
| 7tm_1 | 137.9 | 9.6e−43 | 59 | 341 | 7 transmembrane |
| | | | | | receptor |
| | | | | | (rhodopsin family) |

Hit: name of the domain deduced by the search.
Score: the higher the value, the higher the reliability.
Expect: as the value approaches 0, the reliability becomes higher.
Q from: the start position of the deduced domain.
Q to: the termination position of the deduced domain.
Description: explanation of the deduced domain.

4. Amino Acid Sequence Alignment

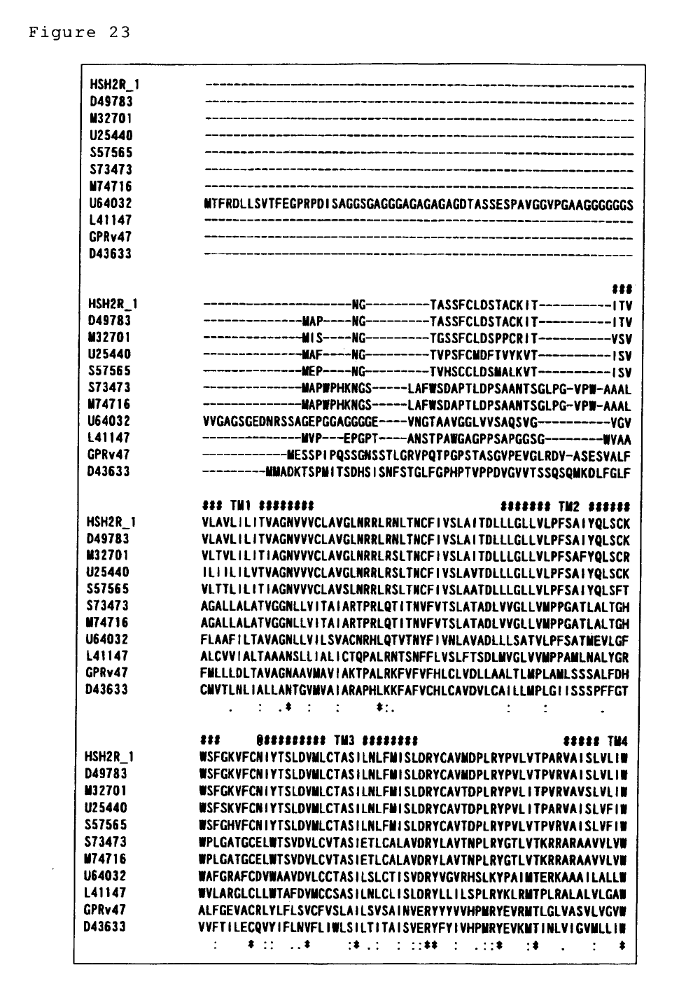

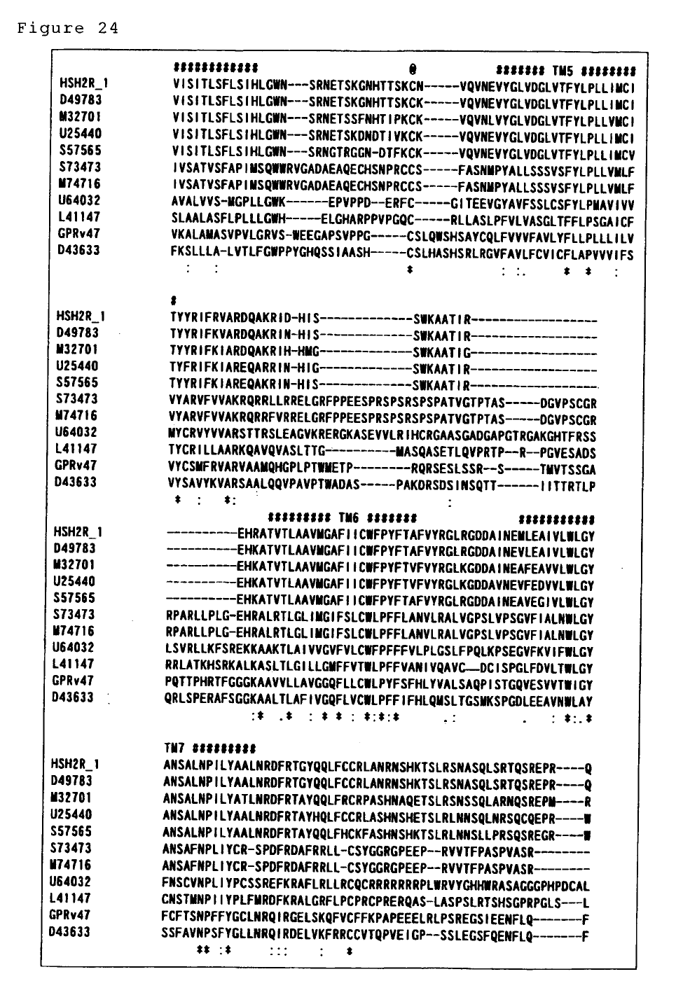

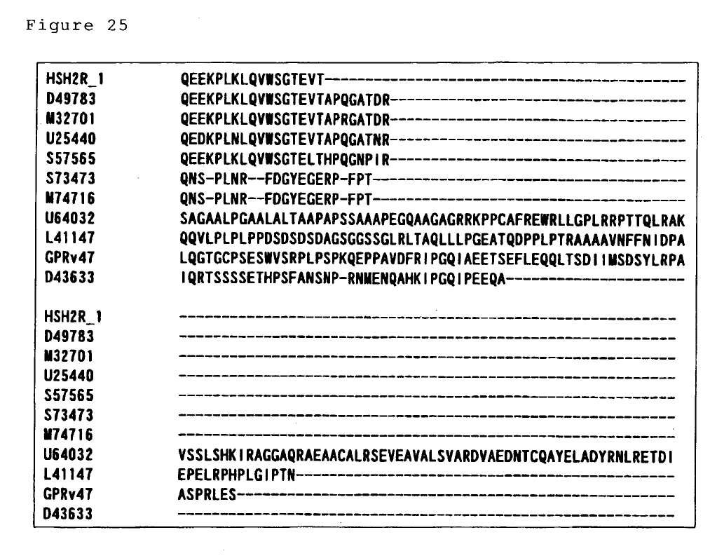

The amino acid sequences of GPRv47 and proteins shown in Table 2 were aligned together by using Clustalw 1.7 (FIGS. 23 to 25). The result showed that GPRv47 comprise seven transmembrane domains (### ###) and Cys (Cys marked with “@”) participating in specific S—S bonding of GPCR.

### EXAMPLE 10

### Analysis of GPRv51 with Bioinformatics

1. Homology Search of GPRv51

The amino acid sequence of GPRv51 was analyzed by searching known sequences (the known sequence databases are produced in EMBL (Release 64, http://www.ebi.ac.uk/), GENBANK (Release 120.0, http://www.ncbi.nlm.nih.gov/), and PIR (Release 66.00, http://www-nbrf.georgetown.edu/pir/)) with an analysis program (BLAST 2.0) (Altschul, Stephen F. et al. (1997) Nucleic Acids Res. 25:3389-3402.). The result showed that GPRv51 had homology to the sequences shown in Table 14. Thus, GPRv51 was revealed to be a novel clone having homology to GPCR. The amino acid sequence of GPRv51 was analyzed by searching known sequences with an analysis program (BLAST 2.0); the result (data with the E-value lower than e-18) is shown in Table 14.

TABLE 14

| Hit | E- | | |
|---|---|---|---|
| (ID) | value | Identities % | Description |
| M35297 | 4e−43 | 36 | G-protein coupled receptor- |
| | | | Rat |
| J03823 | 1e−42 | 34 | Rat mas oncogene, |
| | | | complete cds. |
| M13150 | 3e−40 | 34 | mas proto-oncogene- Human |
| X67735 | 1e−39 | 35 | Mas proto-oncogene- |
| | | | M. musculus mas |
| AL035542 | 1e−35 | 36 | MAS-related Gprotein-coupled |
| | | | receptor MRG- |
| | | | Human |

2. Prediction of Transmembrane Domain

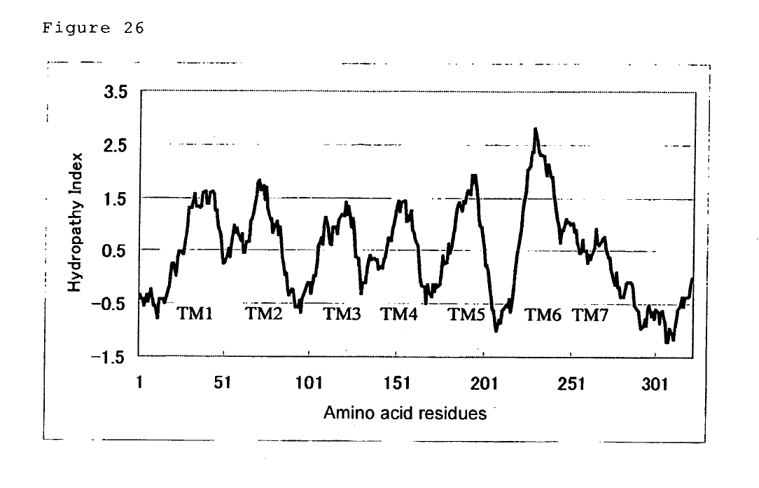

The amino acid sequence of GPRv51 was analyzed according to the method of Kyte-Doolittle (J. Kyte and R. F. Doolittle, (1982), J. Mol. Biol., 157,105-132.), for obtaining a hydropathy plot and used to predict the transmembrane domain. The result showed that GPRv51 had seven transmembrane domains (TM1-TM7) (FIG. 26).

3. HMMPfam Search

Using the amino acid sequence of GPRv51 as the query, PFAM search based on the hidden Markov model (HMMPFAM (Sonnhammer E L, et al., Nucleic Acids Res Jan. 1, 1998; 26 (1):320-322)) was carried out. The search was carried out with the hidden Markov model of HMMER version 2.1 (http://hmmer.wustl.edu/) and the PFAM database of Pfam Version 5.5 (http://www.sanger.ac.uk/Software/Pfam/index.shtml).

The result indicated that GPRv51 comprises tm7_{—}1 (Rhodopsin family). The result of HMMPfam search is shown in Table 15.

TABLE 15

| | | | Q | Q | |
|---|---|---|---|---|---|
| Hit | Score | Expect | from | to | Description |
| 7tm_1 | 32.6 | 1.4e−09 | 44 | 78 | 7 transmembrane |
| | | | | | receptor |
| | | | | | (rhodopsin family) |
| 7tm_1 | 30.1 | 8.7e−09 | 104 | 276 | 7 transmembrane |
| | | | | | receptor |
| | | | | | (rhodopsin family) |

Hit: name of the domain deduced by the search.
Score: the higher the value, the higher the reliability.
Expect: as the value approaches 0, the reliability becomes higher.
Q from: the start position of the deduced domain.
Q to: the termination position of the deduced domain.
Description: explanation of the deduced domain.

4. Amino Acid Sequence Alignment

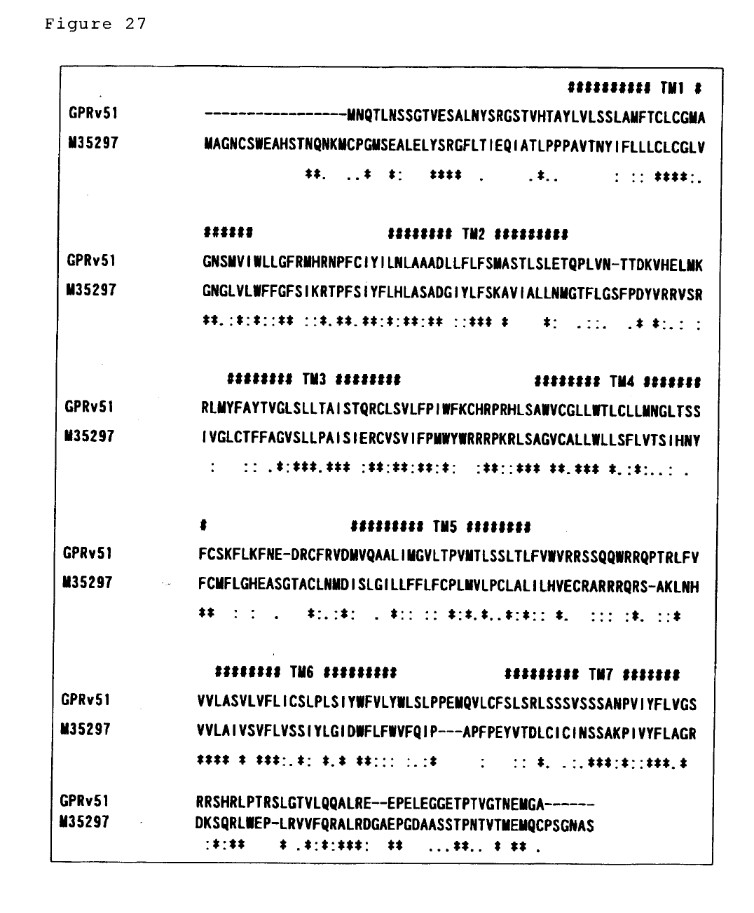

The amino acid sequences of GPRv51 and G-protein coupled receptor—Rat (M35297) were aligned together by using Clustalw 1.7 (FIG. 27). The result showed that GPRv51 comprise seven transmembrane domains (### ###).

### EXAMPLE 11

### Analysis of GPRv71 with Bioinformatics

1. Homology Search of GPRv71

The amino acid sequence of GPRv71 was analyzed by searching known sequences (the known sequence databases are produced in EMBL (Release 64, http://www.ebi.ac.uk/), GENBANK (Release 120.0, http://www.ncbi.nlm.nih.gov/), and PIR (Release 66.00, http://www-nbrf.georgetown.edu/pir/)) with an analysis program (BLAST 2.0) (Altschul, Stephen F. et al. (1997) Nucleic Acids Res. 25:3389-3402.). The result showed that GPRv71 had homology to the sequences shown in Table 16. Thus, GPRv71 was revealed to be a novel clone having homology to GPCR. The amino acid sequence of GPRv71 was analyzed by searching known sequences with an analysis program (BLAST 2.0); the result (data with the E-value lower than e-35) is shown in Table 16.

TABLE 16

| Hit | E- | | |
|---|---|---|---|
| (ID) | value | Identities % | Description |
| AF069555 | 9e−44 | 44 | G protein-coupled |
| | | | receptor p2y3- |
| | | | Meleagris gallopavo |
| X98283 | 9e−44 | 45 | P2Y PURINOCEPTOR 3- |
| | | | G. domesticus |
| AF031897 | 6e−41 | 40 | P2Y nucleotide receptor- |
| | | | Meleagris gallopavo |
| X99953 | 1e−39 | 41 | P2Y PURINOCEPTOR 8- |
| | | | X. laevis |
| D63665 | 2e−37 | 41 | novel G protein-coupled |
| | | | P2 receptor- |
| | | | Rat |
| Y14705 | 1e−36 | 40 | P2Y4 receptor gene- |
| | | | Rattus norvegicus |
| AJ277752 | 2e−36 | 41 | P2Y4 receptor- Mus musculus |

2. Prediction of Transmembrane Domain

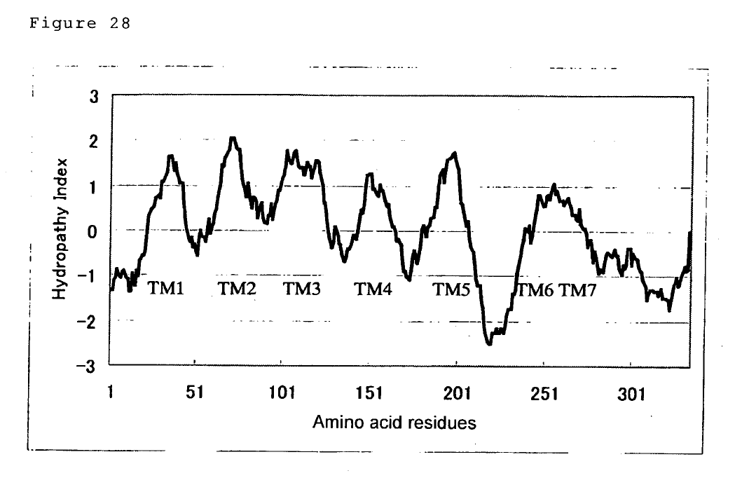

The amino acid sequence of GPRv71 was analyzed according to the method of Kyte-Doolittle (J. Kyte and R. F. Doolittle, (1982), J. Mol. Biol., 157,105-132.), for obtaining a hydropathy plot and used to predict the transmembrane domain. The result showed that GPRv71 had seven transmembrane domains (TM1-TM7) (FIG. 28).

3. HMMPfam Search

Using the amino acid sequence of GPRv71 as the query, PFAM search based on the hidden Markov model (HMMPFAM (Sonnhammer E L, et al., Nucleic Acids Res Jan. 1, 1998; 26 (1):320-322)) was carried out. The search was carried out with the hidden Markov model of HMMER version 2.1 (http://hmmer.wustl.edu/) and the PFAM database of Pfam Version 5.5 (http://www.sanger.ac.uk/Software/Pfam/index.shtml).

The result indicated that GPRv71 comprises tm7_{—}1 (Rhodopsin family). The result of HMMPfam search is shown in Table 17.

TABLE 17

| | | | Q | Q | |
|---|---|---|---|---|---|
| Hit | Score | Expect | from | to | Description |
| 7tm_1 | 90.6 | 7.6e−28 | 40 | 161 | 7 transmembrane |
| | | | | | receptor |
| | | | | | (rhodopsin family) |

Hit: name of the domain deduced by the search.
Score: the higher the value, the higher the reliability.
Expect: as the value approaches 0, the reliability becomes higher.
Q from: the start position of the deduced domain.
Q to: the termination position of the deduced domain.
Description: explanation of the deduced domain.

4. Amino Acid Sequence Alignment

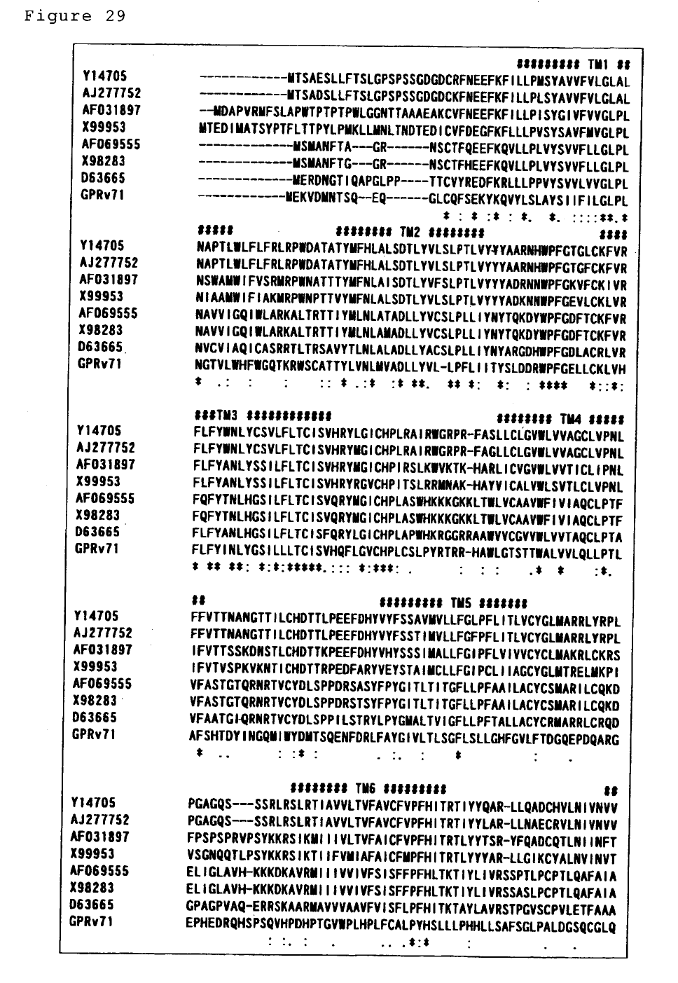

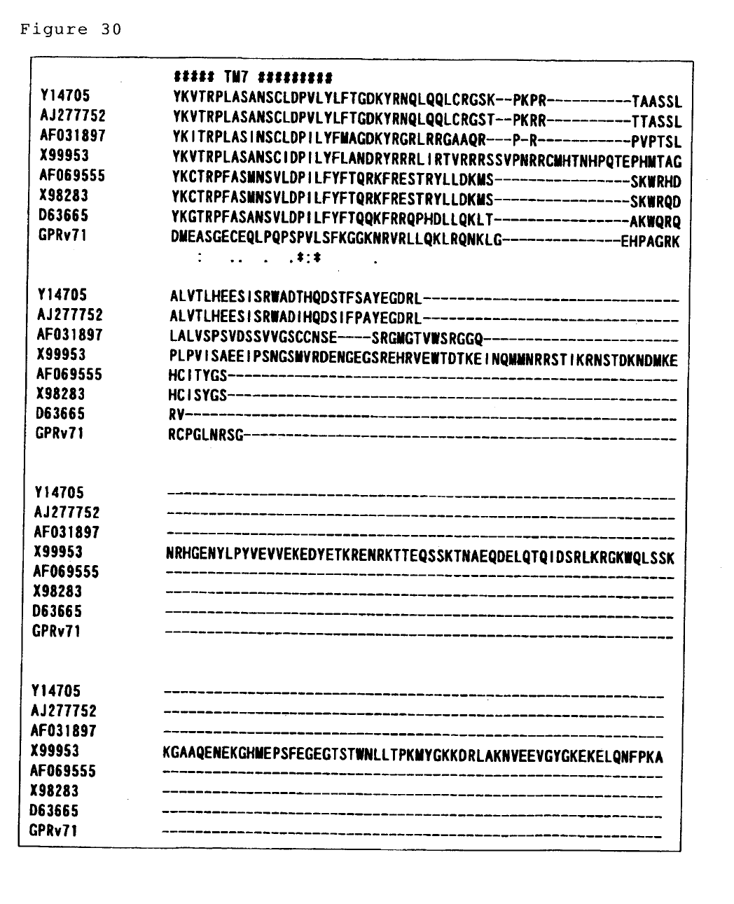

The amino acid sequences of GPRv71 and proteins shown in Table 2 were aligned together by using Clustalw 1.7 (FIGS. 29 and 30). The result showed that GPRv71 comprise seven transmembrane domains (### ###).

### EXAMPLE 12

### Analysis of GPRv72 with Bioinformatics

1. Homology Search of GPRv72

The amino acid sequence of GPRv72 was analyzed by searching known sequences (the known sequence databases are produced in EMBL (Release 64, http://www.ebi.ac.uk/), GENBANK (Release 120.0, http://www.ncbi.nlm.nih.gov/), and PIR (Release 66.00, http://www-nbrf.georgetown.edu/pir/)) with an analysis program (BLAST 2.0) (Altschul, Stephen F. et al. (1997) Nucleic Acids Res. 25:3389-3402.). The result showed that GPRv72 had homology to the sequences shown in Table 18. Thus, GPRv72 was revealed to be a novel clone having homology to GPCR. The amino acid sequence of GPRv72 was analyzed by searching known sequences with an analysis program (BLAST 2.0); the result (data with the E-value lower than e-24) is shown in Table 18.

TABLE 18

| Hit | E- | | |
|---|---|---|---|
| (ID) | value | Identities % | Description |
| AF091890 | 4e−29 | 32 | G-protein coupled |
| | | | receptor RE2- |
| | | | Homo sapiens |
| U81982 | 3e−25 | 30 | alpha 1a-adrenoceptor- |
| | | | Oryctolagus cuniculus |
| S71323 | 6e−25 | 32 | alpha-1A adrenergic receptor- |
| | | | Japanese medaka |
| D63859 | 6e−25 | 32 | alpha1A-adrenoceptor- |
| | | | Oryzias latipes |
| U07126 | 8e−25 | 29 | alpha1c adrenergic receptor- |
| | | | Rattus norvegicus |
| U03866 | 8e−25 | 30 | adrenergic alpha-1c |
| | | | receptor protein- |
| | | | Human |
| AF013261 | 8e−25 | 30 | alpha 1A adrenergic receptor |
| | | | isoform 4- |
| | | | Homo sapiens |
| L31774 | 8e−25 | 30 | alpha-1C-adrenergic receptor- |
| | | | Human |
| D32202 | 8e−25 | 30 | alpha 1C adrenergic |
| | | | receptor isoform 2- |
| | | | Human |
| D32201 | 8e−25 | 30 | alpha 1C adrenergic |
| | | | receptor isoform 3- |
| | | | Human |
| D25235 | 8e−25 | 30 | alpha1C adrenergic receptor |

2. Prediction of Transmembrane Domain

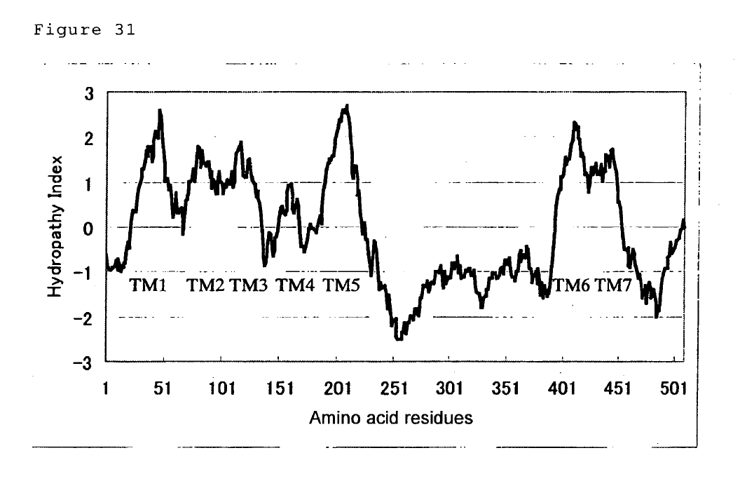

The amino acid sequence of GPRv72 was analyzed according to the method of Kyte-Doolittle (J. Kyte and R. F. Doolittle, (1982), J. Mol. Biol., 157,105-132.), for obtaining a hydropathy plot and used to predict the transmembrane domain. The result showed that GPRv72 had seven transmembrane domains (TM1-TM7) (FIG. 31).

3. HMMPfam Search

Using the amino acid sequence of GPRv72 as the query, PFAM search based on the hidden Markov model (HMMPFAM (Sonnhammer E L, et al., Nucleic Acids Res Jan. 1, 1998; 26 (1):320-322)) was carried out. The search was carried out with the hidden Markov model of HMMER version 2.1 (http://hmmer.wustl.edu/) and the PFAM database of Pfam Version 5.5 (http://www.sanger.ac.uk/Software/Pfam/index.shtml).

The result indicated that GPRv72 comprises tm7_{—}1 (Rhodopsin family). The result of HMMPfam search is shown in Table 19.

TABLE 19

| Hit | Score | Expect | Q from | Q to | Description |
|---|---|---|---|---|---|
| 7tm_1 | 196.1 | 4.7e−61 | 48 | 454 | 7 transmembrane |
| | | | | | receptor |
| | | | | | (rhodopsin family) |

Hit: name of the domain deduced by the search.
Score: the higher the value, the higher the reliability.
Expect: as the value approaches 0, the reliability becomes higher.
Q from: the start position of the deduced domain.
Q to: the termination position of the deduced domain.
Description: explanation of the deduced domain.

4. Amino Acid Sequence Alignment

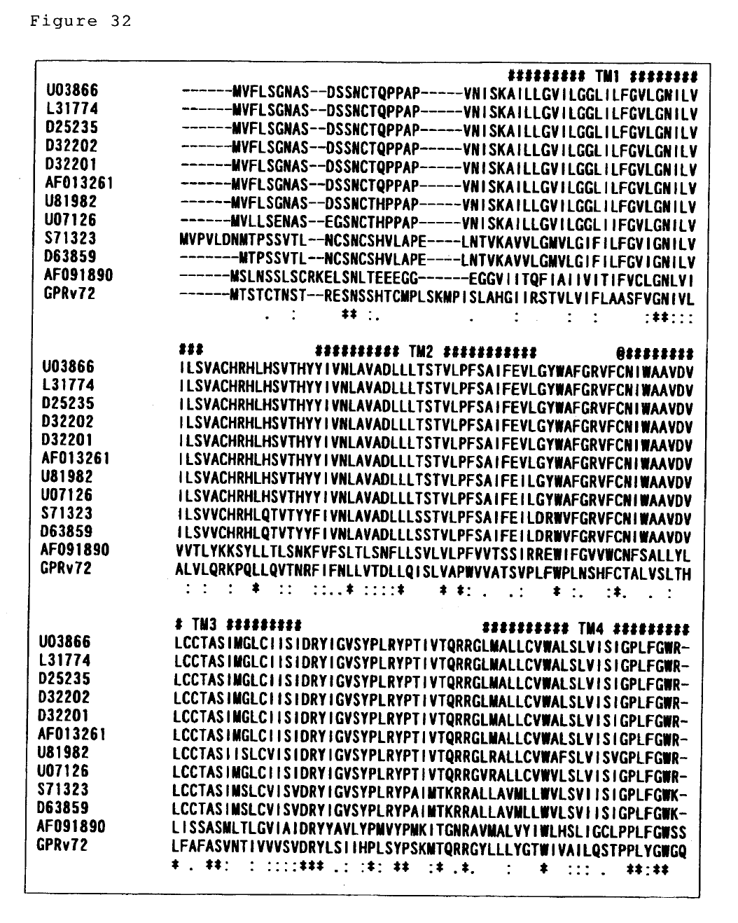

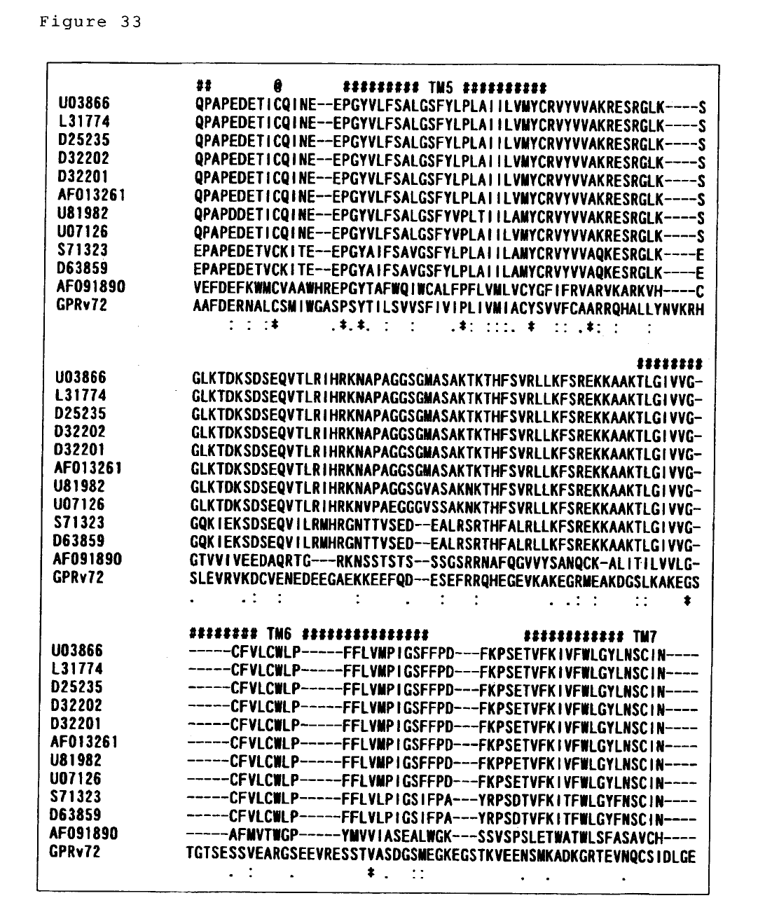

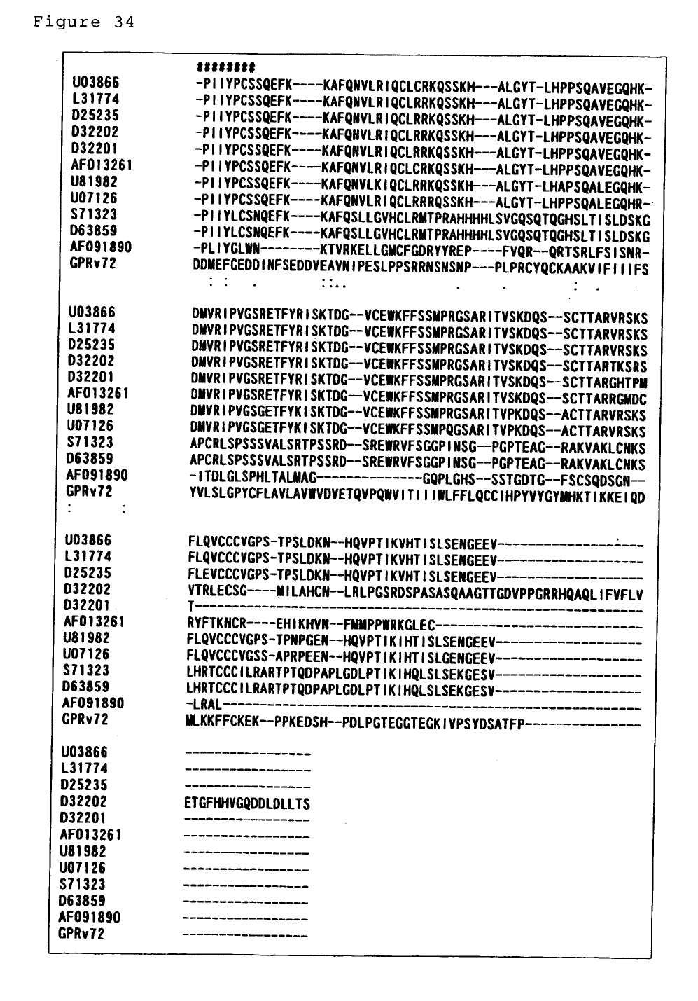

The amino acid sequences of GPRv72 and proteins shown in Table 18 were aligned together by using Clustalw 1.7 (FIGS. 32 to 34). The result showed that GPRv72 comprise seven transmembrane domains (### ###) and Cys (Cys marked with “@”) participating in specific S—S bonding of GPCR.

Industrial Applicability

The present invention provided novel G protein-coupled receptors (GPRv8, GPRv12, GPRv16, GPRv21, GPRv40, GPRv47, GPRv51, GPRv71, and GPRv72), the genes encoding the proteins, vectors containing the genes, host cells containing the vectors, and a method for producing the proteins. Further, the present invention provided a screening method for compounds modifying the activities of the proteins. The proteins and genes of the present invention, and compounds modifying the activity of the proteins, are expected to be used for the development of new preventives and therapeutics for the diseases, with which the G protein-coupled receptors of the present invention are associated.

1. A DNA that encodes a guanosine triphosphate-binding protein-coupled receptor, wherein said DNA is selected from the group consisting of the following (a) to (d):
(a) a DNA encoding a protein comprising the amino acid sequence of any one of SEQ ID NOs: 1 to 4 and 17 to 21;
(b) a DNA comprising a coding region of the nucleotide sequence of any one of SEQ ID NOs: 5 to 8 and 22 to 26;
(c) a DNA encoding a protein comprising the amino acid sequence of any one of SEQ ID NOs: 1 to 4 and 17 to 21 in which one or more amino acids are substituted, deleted, added, and/or inserted; and
(d) a DNA hybridizing under stringent conditions to the DNA comprising the nucleotide sequence of any one of SEQ ID NOs: 5 to 8 and 22 to 26.

2. A DNA encoding a partial peptide of a protein comprising the amino acid sequence of any one of SEQ ID NOs: 1 to 4 and 17 to 21:

3. A vector comprising the DNA of any one of claims 1 and 2.

4. A transformant carrying the DNA of any one of claims 1 and 2 or the vector of claim 3.

5. A protein or a peptide encoded by the DNA of any one of claims 1 and 2.

6. A method for producing the protein or the peptide of claim 5, said method comprising the steps of culturing the transformant of claim 4 and recovering an expressed protein or peptide from the transformant or culture supernatant thereof.

7. A method of screening for ligands that bind to the protein of claim 5, said method comprising the steps of:
(a) contacting a test sample with the protein or the peptide of claim 5; and
(b) selecting compounds that binds to said protein or said peptide.

8. A method of screening for compounds that have activity of inhibiting the binding between the protein of claim 5 and a ligand thereof, said method comprising the steps of:
(a) contacting the protein of claim 5 or a partial peptide thereof with the ligand in the presence of a test sample and detecting a binding activity of said protein or said partial peptide with said ligand; and
(b) selecting compounds that reduces the binding activity detected in step (a) as compared with a binding activity detected in the absence of the test sample;

9. A method of screening for compounds that inhibit or enhance activity of the protein of claim 5, said method comprising the steps of:
(a) contacting a ligand of said protein with cells expressing said protein in the presence of a test sample;
(b) detecting an alteration in the cells that results from binding of said ligand to said protein; and
(c) selecting compounds that suppress or enhance the alteration detected in step (b) as compared with an alteration detected in the cells in the absence of the test sample.

10. The method of claims 8 or 9, wherein the alteration in cells is a change in cAMP concentration or calcium concentration.

11. An antibody binding to the protein of claim 5.

12. A compound isolated by the method of any one of claim 7 to 10.

13. A pharmaceutical composition comprising the compound of claim 12 as an active ingredient.

14. The pharmaceutical composition of claim 13, wherein said pharmaceutical composition is formulated for the treatment of a disease selected from the group consisting of cancer, cirrhosis, and Alzheimer's disease.

15. A polynucleotide comprising at least 15 nucleotides, wherein said polynucleotide is complementary to the DNA comprising the nucleotide sequence of any one of SEQ ID NOs: 5 to 8 and 22 to 26 or a complementary strand thereof.

16. A method for diagnosing a disease selected from the group consisting of cancer, cirrhosis, and Alzheimer's disease, said method comprising the steps of detecting expression of the DNA of claim 1 in tissues related to the disease derived from a subject, or mutation in the DNA of claim 1 in the subject.

17. An agent for diagnosing a disease selected from the group consisting of cancer, cirrhosis, and Alzheimer's disease, said agent comprising the antibody of claim 11 or the nucleotide of claim 15.


SEQUENCE LISTING (63 sequences)

SEQ ID NO: 1 - protein, 371 aa, Homo sapiens
MPANFTEGSFDSSGTGQTLDSSPVACTETVTFTEVVEGKEWGSFYYSFKTEQLITLWVLFVFTIVGNSVVLFSTWRRKKKSRMTFFVTQLAITDSFTGLVNILTDINWRFTGDFTAPDLVCRVVRYLQVVLLYASTYVLVSLSIDRYHAIVYPMKFLQGEKQARVLIVIAWSLSFLFSIPTLIIFGKRTLSNGEVQCWALWPDDSYWTPYMTIVAFLVYFIPLTIISIMYGIVIRTIWIKSKTYETVISNCSDGKLCSSYNRGLISKAKIKAIKYSIIIILAFICCWSPYFLFDILDNFNLLPDTQERFYASVIIQNLPALNSAINPLIYCVFSSSISFPCREQRSQDSRMTFRERTERHEMQILSKPEFI

SEQ ID NO: 2 - protein, 363 aa, Homo sapiens
MGPGEALLAGLLVMVLAVALLSNALVLLCCAYSAELRTRASGVLLVNLSLGHLLLAALDMPFTLLGVMRGRTPSAPGACQVIGFLDTFLASNAALSVAALSADQWLAVGFPLRYAGRLRPRYAGLLLGCAWGQSLAFSGAALGCSWLGYSSAFASCSLRLPPEPERPRFAAFTATLHAVGFVLPLAVLCLTSLQVHRVARRHCQRMDTVTMKALALLADLHPSVRQRCLIQQKRRRHRATRKIGIAIATFLICFAPYVMTRLAELVPFVTVNAQWGILSKCLTYSKAVADPFTYSLLRRPFRQVLAGMVHRLLKRTPRPASTHDSSLDVAGMVHQLLKRTPRPASTHNGSVDTENDSCLQQTH

SEQ ID NO: 3 - protein, 419 aa, Homo sapiens
MLAAAFADSNSSSMNVSFAHLHFAGGYLPSDSQDWRTIIPALLVAVCLVGFVGNLCVIGILLHNAWKGKPSMIHSLILNLSLADLSLLLFSAPIRATAYSKSVWDLGWFVCKSSDWFIHTCMAAKSLTIVVVAKVCFMYASDPAKQVSIHNYTIWSVLVAIWTVASLLPLPEWFFSTIRHHEGVEMCLVDVPAVAEEFMSMFGKLYPLLAFGLPLFFASFYFWRAYDQCKKRGTKTQNLRNQIRSKQVTVMLLSIAIISAVLWLPEWVAWLWVWHLKAAGPAPPQGFIALSQVLMFSISSANPLIFLVMSEEFREGLKGVWKWMITKKPPTVSESQETPAGNSEGLPDKVPSPESPASIPEKEKPSSPSSGKGKTEKAEIPILPDVEQFWHERDTVPSVQDNDPIPWEHEDQETGEGVK

SEQ ID NO: 4 - protein, 393 aa, Homo sapiens
METTMGFMDDNATNTSTSFLSVLNPHGAHATSFPFNFSYSDYDMPLDEDEDVTNSRTFFAAKIVIGMALVGIMLVCGIGNFIFIAALVRYKKLRNLTNLLIANLAISDFLVAIVCCPFEMDYYVVRQLSWEHGHVLCTSVNYLRTVSLYVSTNALLAIAIDRYLAIVHPLRPRMKCQTATGLIALVWTVSILIAIPSAYFTTETVLVIVKSQEKIFCGQIWPVDQQLYYKSYFLFIFGIEFVGPVVTMTLCYARISRELWFKAVPGFQTEQIRKRLRCRRKTVLVLMCILTAYVLCWAPFYGFTIVRDFFPTVFVKEKHYLTAFYIVECIAMSNSMINTLCFVTVKNDTVKYFKKIMLLHWKASYNGGKSSADLDLKTIGMPATEEVDCIRLK

SEQ ID NO: 5 - DNA, 1116 bp, Homo sapiens
atgccagccaacttcacagagggcagcttcgattccagtgggaccgggcagacgctggattcttccccagtggcttgcactgaaacagtgacttttactgaagtggtggaaggaaaggaatggggttccttctactactcctttaagactgagcaattgataactctgtgggtcctctttgtttttaccattgttggaaactccgttgtgcttttttccacatggaggagaaagaagaagtcaagaatgaccttctttgtgactcagctggccatcacagattctttcacaggactggtcaacatcttgacagatattaattggcgattcactggagacttcacggcacctgacctggtttgccgagtggtccgctatttgcaggttgtgctgctctacgcctctacctacgtcctggtgtccctcagcatagacagataccatgccatcgtctaccccatgaagttccttcaaggagaaaagcaagccagggtcctcattgtgatcgcctggagcctgtcttttctgttctccattcccaccctgatcatatttgggaagaggacactgtccaacggtgaagtgcagtgctgggccctgtggcctgacgactcctactggaccccatacatgaccatcgtggccttcctggtgtacttcatccctctgacaatcatcagcatcatgtatggcattgtgatccgaactatttggattaaaagcaaaacctacgaaacagtgatttccaactgctcagatgggaaactgtgcagcagctataaccgaggactcatctcaaaggcaaaaatcaaggctatcaagtatagcatcatcatcattcttgccttcatctgctgttggagtccatacttcctgtttgacattttggacaatttcaacctccttccagacacccaggagcgtttctatgcctctgtgatcattcagaacctgccagcattgaatagtgccatcaaccccctcatctactgtgtcttcagcagctccatctctttcccctgcagggagcaaagatcacaggattccagaatgacgttccgggagagaactgagaggcatgagatgcagattctgtccaagccagaattcatctag

SEQ ID NO: 6 - DNA, 1092 bp, Homo sapiens
atgggccccggcgaggcgctgctggcgggtctcctggtgatggtactggccgtggcgctgctatccaacgcactggtgctgctttgttgcgcctacagcgctgagctccgcactcgagcctcaggcgtcctcctggtgaatctgtctctgggccacctgctgctggcggcgctggacatgcccttcacgctgctcggtgtgatgcgcgggcggacaccgtcggcgcccggcgcatgccaagtcattggcttcctggacaccttcctggcgtccaacgcggcgctgagcgtggcggcgctgagcgcagaccagtggctggcagtgggcttcccactgcgctacgccggacgcctgcgaccgcgctatgccggcctgctgctgggctgtgcctggggacagtcgctggccttctcaggcgctgcacttggctgctcgtggcttggctacagcagcgccttcgcgtcctgttcgctgcgcctgccgcccgagcctgagcgtccgcgcttcgcagccttcaccgccacgctccatgccgtgggcttcgtgctgccgctggcggtgctctgcctcacctcgctccaggtgcaccgggtggcacgcagacactgccagcgcatggacaccgtcaccatgaaggcgctcgcgctgctcgccgacctgcaccccagtgtgcggcagcgctgcctcatccagcagaagcggcgccgccaccgcgccaccaggaagattggcattgctattgcgaccttcctcatctgctttgccccgtatgtcatgaccaggctggcggagctcgtgcccttcgtcaccgtgaacgcccagtggggcatcctcagcaagtgcctgacctacagcaaggcggtggccgacccgttcacgtactctctgctccgccggccgttccgccaagtcctggccggcatggtgcaccggctgctgaagagaaccccgcgcccagcatccacccatgacagctctctggatgtggccggcatggtgcaccagctgctgaagagaaccccgcgcccagcgtccacccacaacggctctgtggacacagagaatgattcctgcctgcagcagacacactga

SEQ ID NO: 7 - DNA, 1260 bp, Homo sapiens
atgctggcagctgcctttgcagactctaactccagcagcatgaatgtgtcctttgctcacctccactttgccggagggtacctgccctctgattcccaggactggagaaccatcatcccggctctcttggtggctgtctgcctggtgggcttcgtgggaaacctgtgtgtgattggcatcctccttcacaatgcttggaaaggaaagccatccatgatccactccctgattctgaatctcagcctggctgatctctccctcctgctgttttctgcacctatccgagctacggcgtactccaaaagtgtttgggatctaggctggtttgtctgcaagtcctctgactggtttatccacacatgcatggcagccaagagcctgacaatcgttgtggtggccaaagtatgcttcatgtatgcaagtgacccagccaagcaagtgagtatccacaactacaccatctggtcagtgctggtggccatctggactgtggctagcctgttacccctgccggaatggttctttagcaccatcaggcatcatgaaggtgtggaaatgtgcctcgtggatgtaccagctgtggctgaagagtttatgtcgatgtttggtaagctctacccactcctggcatttggccttccattattttttgccagcttttatttctggagagcttatgaccaatgtaaaaaacgaggaactaagactcaaaatcttagaaaccagatacgctcaaagcaagtcacagtgatgctgctgagcattgccatcatctctgctgtcttgtggctccccgaatgggtagcttggctgtgggtatggcatctgaaggctgcaggcccggccccaccacaaggtttcatagccctgtctcaagtcttgatgttttccatctcttcagcaaatcctctcatttttcttgtgatgtcggaagagttcagggaaggcttgaaaggtgtatggaaatggatgataaccaaaaaacctccaactgtctcagagtctcaggaaacaccagctggcaactcagagggtcttcctgacaaggttccatctccagaatccccagcatccataccagaaaaagagaaacccagctctccctcctctggcaaagggaaaactgagaaggcagagattcccatccttcctgacgtagagcagttttggcatgagagggacacagtcccttctgtacaggacaatgaccctatcccctgggaacatgaagatcaagagacaggggaaggtgttaaatag

SEQ ID NO: 8 - DNA, 1182 bp, Homo sapiens
atggagaccaccatggggttcatggatgacaatgccaccaacacttccaccagcttcctttctgtgctcaaccctcatggagcccatgccacttccttcccattcaacttcagctacagcgactatgatatgcctttggatgaagatgaggatgtgaccaattccaggacgttctttgctgccaagattgtcattgggatggccctggtgggcatcatgctggtctgcggcattggaaacttcatctttatcgctgccctggtccgctacaagaaactgcgcaacctcaccaacctgctcatcgccaacctggccatctctgacttcctggtggccattgtctgctgcccctttgagatggactactatgtggtgcgccagctctcctgggagcacggccacgtcctgtgcacctctgtcaactacctgcgcactgtctctctctatgtctccaccaatgccctgctggccatcgccattgacaggtatctggctattgtccatccgctgagaccacggatgaagtgccaaacagccactggcctgattgccttggtgtggacggtgtccatcctgatcgccatcccttccgcctacttcaccaccgagacggtcctcgtcattgtcaagagccaggaaaagatcttctgcggccagatctggcctgtggaccagcagctctactacaagtcctacttcctctttatctttggcatagaattcgtgggccccgtggtcaccatgaccctgtgctatgccaggatctcccgggagctctggttcaaggcggtccctggattccagacagagcagatccgcaagaggctgcgctgccgcaggaagacggtcctggtgctcatgtgcatcctcaccgcctacgtgctatgctgggcgcccttctacggcttcaccatcgtgcgcgacttcttccccaccgtgtttgtgaaggagaagcactacctcactgccttctacatcgtcgagtgcatcgccatgagcaacagcatgatcaacactctgtgcttcgtgaccgtcaagaacgacaccgtcaagtacttcaaaaagatcatgttgctccactggaaggcttcttacaatggcggtaagtccagtgcagacctggacctcaagacaattgggatgcctgccaccgaagaggtggactgcatcagactaaaataa

SEQ ID NO: 9 - DNA, 28 bp, Artificial Sequence
  Description of Artificial Sequencean artificially synthesized primer sequence
atgccagccaacttcacagagggcagct

SEQ ID NO: 10 - DNA, 28 bp, Artificial Sequence
  Description of Artificial Sequencean artificially synthesized primer sequence
ctagatgaattctggcttggacagaatc

SEQ ID NO: 11 - DNA, 28 bp, Artificial Sequence
  Description of Artificial Sequencean artificially synthesized primer sequence
atgggccccggcgaggcgctgctggcgg

SEQ ID NO: 12 - DNA, 28 bp, Artificial Sequence
  Description of Artificial Sequencean artificially synthesized primer sequence
tcagtgtgtctgctgcaggcaggaatca

SEQ ID NO: 13 - DNA, 30 bp, Artificial Sequence
  Description of Artificial Sequencean artificially synthesized primer sequence
atgctggcagctgcctttgcagactctaac

SEQ ID NO: 14 - DNA, 30 bp, Artificial Sequence
  Description of Artificial Sequencean artificially synthesized primer sequence
ctatttaacaccttcccctgtctcttgatc

SEQ ID NO: 15 - DNA, 28 bp, Artificial Sequence
  Description of Artificial Sequencean artificially synthesized primer sequence
atggagaccaccatggggttcatggatg

SEQ ID NO: 16 - DNA, 30 bp, Artificial Sequence
  Description of Artificial Sequencean artificially synthesized primer sequence
ttattttagtctgatgcagtccacctcttc

SEQ ID NO: 17 - protein, 434 aa, Homo sapiens [sequence omitted]

SEQ ID NO: 18 - protein, 451 aa, Homo sapiens
MESSPIPQSSGNSSTLGRVPQTPGPSTASGVPEVGLRDVASESVALFFMLLLDLTAVAGNAAVMAVIAKTPALRKFVFVFHLCLVDLLAALTLMPLAMLSSSALFDHALFGEVACRLYLFLSVCFVSLAILSVSAINVERYYYVVHPMRYEVRMTLGLVASVLVGVWVKALAMASVPVLGRVSWEEGAPSVPPGCSLQWSHSAYCQLFVVVFAVLYFLLPLLLILVVYCSMFRVARVAAMQHGPLPTWMETPRQRSESLSSRSTMVTSSGAPQTTPHRTFGGGKAAVVLLAVGGQFLLCWLPYFSFHLYVALSAQPISTGQVESVVTWIGYFCFTSNPFFYGCLNRQIRGELSKQFVCFFKPAPEEELRLPSREGSIEENFLQFLQGTGCPSESWVSRPLPSPKQEPPAVDFRIPGQIAEETSEFLEQQLTSDIIMSDSYLRPAASPRLES

SEQ ID NO: 19 - protein, 321 aa, Homo sapiens
MNQTLNSSGTVESALNYSRGSTVHTAYLVLSSLAMFTCLCGMAGNSMVIWLLGFRMHRNPFCIYILNLAAADLLFLFSMASTLSLETQPLVNTTDKVHELMKRLMYFAYTVGLSLLTAISTQRCLSVLFPIWFKCHRPRHLSAWVCGLLWTLCLLMNGLTSSFCSKFLKFNEDRCFRVDMVQAALIMGVLTPVMTLSSLTLFVWVRRSSQQWRRQPTRLFVVVLASVLVFLICSLPLSIYWFVLYWLSLPPEMQVLCFSLSRLSSSVSSSANPVIYFLVGSRRSHRLPTRSLGTVLQQALREEPELEGGETPTVGTNEMGA

SEQ ID NO: 20 - protein, 333 aa, Homo sapiens
MEKVDMNTSQEQGLCQFSEKYKQVYLSLAYSIIFILGLPLNGTVLWHFWGQTKRWSCATTYLVNLMVADLLYVLLPFLIITYSLDDRWPFGELLCKLVHFLFYINLYGSILLLTCISVHQFLGVCHPLCSLPYRTRRHAWLGTSTTWALVVLQLLPTLAFSHTDYINGQMIWYDMTSQENFDRLFAYGIVLTLSGFLSLLGHFGVLFTDGQEPDQARGEPHEDRQHSPSQVHPDHPTGVWPLHPLFCALPYHSLLLPHHLLSAFSGLPALDGSQCGLQDMEASGECEQLPQPSPVLSFKGGKNRVRLLQKLRQNKLGEHPAGRKRCPGLNRSG

SEQ ID NO: 21 - protein, 508 aa, Homo sapiens
MTSTCTNSTRESNSSHTCMPLSKMPISLAHGIIRSTVLVIFLAASFVGNIVLALVLQRKPQLLQVTNRFIFNLLVTDLLQISLVAPWVVATSVPLFWPLNSHFCTALVSLTHLFAFASVNTIVVVSVDRYLSIIHPLSYPSKMTQRRGYLLLYGTWIVAILQSTPPLYGWGQAAFDERNALCSMIWGASPSYTILSVVSFIVIPLIVMIACYSVVFCAARRQHALLYNVKRHSLEVRVKDCVENEDEEGAEKKEEFQDESEFRRQHEGEVKAKEGRMEAKDGSLKAKEGSTGTSESSVEARGSEEVRESSTVASDGSMEGKEGSTKVEENSMKADKGRTEVNQCSIDLGEDDMEFGEDDINFSEDDVEAVNIPESLPPSRRNSNSNPPLPRCYQCKAAKVIFIIIFSYVLSLGPYCFLAVLAVWVDVETQVPQWVITIIIWLFFLQCCIHPYVYGYMHKTIKKEIQDMLKKFFCKEKPPKEDSHPDLPGTEGGTEGKIVPSYDSATFP

SEQ ID NO: 22 - DNA, 1305 bp, Homo sapiens
atggaggatctctttagcccctcaattctgccgccggcgcccaacatttccgtgcccatcttgctgggctggggtctcaacctgaccttggggcaaggagcccctgcctctgggccgcccagccgccgcgtccgcctggtgttcctgggggtcatcctggtggtggcggtggcaggcaacaccacagtgctgtgccgcctgtgcggcggcggcgggccctgggcgggccccaagcgtcgcaagatggacttcctgctggtgcagctggccctggcggacctgtacgcgtgcgggggcacggcgctgtcacagctggcctgggaactgctgggcgagccccgcgcggccacgggggacctggcgtgccgcttcctgcagctgctgcaggcatccgggcggggcgcctcggcccacctcgtggtgctcatcgccctcgagcgccggcgcgcggtgcgtcttccgcacggccggccgctgcccgcgcgtgccctcgccgccctgggctggctgctggcactgctgctggcgctgcccccggccttcgtggtgcgcggggactccccctcgccgctgccgccgccgccgccgccaacgtccctgcagccaggcgcgcccccggccgcccgcgcctggccggggcagcgtcgctgccacgggatcttcgcgcccctgccgcgctggcacctgcaggtctacgcgttctacgaggccgtcgcgggcttcgtcgcgcctgttacggtcctgggcgtcgcttgcggccacctactctccgtctggtggcggcaccggccgcaggcccccgcggctgcagcgccctggtcggcgagcccaggtcgagcccctgcgcccagcgcgctgccccgcgccaaggtgcagagcctgaagatgagcctgctgctggcgctgctgttcgtgggctgcgagctgccctactttgccgcccggctggcggccgcgtggtcgtccgggcccgcgggagactgggagggagagggcctgtcggcggcgctgcgcgtggtggcgatggccaacagcgctctcaatcccttcgtctacctcttcttccaggcgggcgactgccggctccggcgacagctgcggaagcggctgggctctctgtgctgcgcgccgcagggaggcgcggaggacgaggaggggccccggggccaccaggcgctctaccgccaacgctggccccaccctcattatcaccatgctcggcgggaaccgctggacgagggcggcttgcgcccaccccctccgcgccccagacccctgccttgctcctgcgaaagtgccttctag

SEQ ID NO: 23 - DNA, 1356 bp, Homo sapiens
atggagtcctcacccatcccccagtcatcagggaactcttccactttggggagggtccctcaaaccccaggtccctctactgccagtggggtcccggaggtggggctacgggatgttgcttcggaatctgtggccctcttcttcatgctcctgctggacttgactgctgtggctggcaatgccgctgtgatggccgtgatcgccaagacgcctgccctccgaaaatttgtcttcgtcttccacctctgcctggtggacctgctggctgccctgaccctcatgcccctggccatgctctccagctctgccctctttgaccacgccctctttggggaggtggcctgccgcctctacttgtttctgagcgtgtgctttgtcagcctggccatcctctcggtgtcagccatcaatgtggagcgctactattacgtagtccaccccatgcgctacgaggtgcgcatgacgctggggctggtggcctctgtgctggtgggtgtgtgggtgaaggccttggccatggcttctgtgccagtgttgggaagggtctcctgggaggaaggagctcccagtgtccccccaggctgttcactccagtggagccacagtgcctactgccagctttttgtggtggtctttgctgtcctttactttctgttgcccctgctcctcatacttgtggtctactgcagcatgttccgagtggcccgcgtggctgccatgcagcacgggccgctgcccacgtggatggagacaccccggcaacgctccgaatctctcagcagccgctccacgatggtcaccagctcgggggccccccagaccaccccacaccggacgtttgggggagggaaagcagcagtggttctcctggctgtggggggacagttcctgctctgttggttgccctacttctctttccacctctatgttgccctgagtgctcagcccatttcaactgggcaggtggagagtgtggtcacctggattggctacttttgcttcacttccaaccctttcttctatggatgtctcaaccggcagatccggggggagctcagcaagcagtttgtctgcttcttcaagccagctccagaggaggagctgaggctgcctagccgggagggctccattgaggagaacttcctgcagttccttcaggggactggctgtccttctgagtcctgggtttcccgacccctacccagccccaagcaggagccacctgctgttgactttcgaatcccaggccagatagctgaggagacctctgagttcctggagcagcaactcaccagcgacatcatcatgtcagacagctacctccgtcctgccgcctcaccccggctggagtcatga

SEQ ID NO: 24 - DNA, 966 bp, Homo sapiens
atgaaccagactttgaatagcagtgggaccgtggagtcagccctaaactattccagagggagcacagtgcacacggcctacctggtgctgagctccctggccatgttcacctgcctgtgcgggatggcaggcaacagcatggtgatctggctgctgggctttcgaatgcacaggaaccccttctgcatctatatcctcaacctggcggcagccgacctcctcttcctcttcagcatggcttccacgctcagcctggaaacccagcccctggtcaataccactgacaaggtccacgagctgatgaagagactgatgtactttgcctacacagtgggcctgagcctgctgacggccatcagcacccagcgctgtctctctgtcctcttccctatctggttcaagtgtcaccggcccaggcacctgtcagcctgggtgtgtggcctgctgtggacactctgtctcctgatgaacgggttgacctcttccttctgcagcaagttcttgaaattcaatgaagatcggtgcttcagggtggacatggtccaggccgccctcatcatgggggtcttaaccccagtgatgactctgtccagcctgaccctctttgtctgggtgcggaggagctcccagcagtggcggcggcagcccacacggctgttcgtggtggtcctggcctctgtcctggtgttcctcatctgttccctgcctctgagcatctactggtttgtgctctactggttgagcctgccgcccgagatgcaggtcctgtgcttcagcttgtcacgcctctcctcgtccgtaagcagcagcgccaaccccgtcatctacttcctggtgggcagccggaggagccacaggctgcccaccaggtccctggggactgtgctccaacaggcgcttcgcgaggagcccgagctggaaggtggggagacgcccaccgtgggcaccaatgagatgggggcttga

SEQ ID NO: 25 - DNA, 1002 bp, Homo sapiens
atggagaaggtggacatgaatacatcacaggaacaaggtctctgccagttctcagagaagtacaagcaagtctacctctccctggcctacagtatcatctttatcctagggctgccactaaatggcactgtcttgtggcacttctggggccaaaccaagcgctggagctgtgccaccacctatctggtgaacctgatggtggccgacctgctttatgtgctattgcccttcctcatcatcacctactcactagatgacaggtggcccttcggggagctgctctgcaagctggtgcacttcctgttctatatcaacctttacggcagcatcctgctgctgacctgcatctctgtgcaccagttcctaggtgtgtgccacccactgtgttcgctgccctaccggacccgcaggcatgcctggctgggcaccagcaccacctgggccctggtggtcctccagctgctgcccacactggccttctcccacacggactacatcaatggccagatgatctggtatgacatgaccagccaagagaattttgatcggctttttgcctacggcatagttctgacattgtctggctttctttccctccttggtcattttggtgtgctattcactgatggtcaggagcctgatcaagccagaggagaacctcatgaggacaggcaacacagcccgagccaggtccatccggaccatcctactggtgtgtggcctcttcaccctctgttttgtgcccttccatatcactcgctccttctacctcaccatctgctttctgctttctcaggactgccagctcttgatggcagccagtgtggcctacaagatatggaggcctctggtgagtgtgagcagctgcctcaacccagtcctgtactttctttcaaggggggcaaaaatagagtcaggctcctccagaaactgaggcagaacaagttgggtgagcatccagctgggaggaagagatgcccagggttgaacagatctgggtaa

SEQ ID NO: 26 - DNA, 1527 bp, Homo sapiens
atgacgtccacctgcaccaacagcacgcgcgagagtaacagcagccacacgtgcatgcccctctccaaaatgcccatcagcctggcccacggcatcatccgctcaaccgtgctggttatcttcctcgccgcctctttcgtcggcaacatagtgctggcgctagtgttgcagcgcaagccgcagctgctgcaggtgaccaaccgttttatctttaacctcctcgtcaccgacctgctgcagatttcgctcgtggccccctgggtggtggccacctctgtgcctctcttctggcccctcaacagccacttctgcacggccctggttagcctcacccacctgttcgccttcgccagcgtcaacaccattgtcttggtgtcagtggatcgctacttgtccatcatccaccctctctcctacccgtccaagatgacccagcgccgcggttacctgctcctctatggcacctggattgtggccatcctgcagagcactcctccactctacggctggggccaggctgcctttgatgagcgcaatgctctctgctccatgatctggggggccagccccagctacactattctcagcgtggtgtccttcatcgtcattccactgattgtcatgattgcctgctactccgtggtgttctgtgcagcccggaggcagcatgctctgctgtacaatgtcaagagacacagcttggaagtgcgagtcaaggactgtgtggagaatgaggatgaagagggagcagagaagaaggaggagttccaggatgagagtgagtttcgccgccagcatgaaggtgaggtcaaggccaaggagggcagaatggaagccaaggacggcagcctgaaggccaaggaaggaagcacggggaccagtgagagtagtgtagaggccaggggcagcgaggaggtcagagagagcagcacggtggccagcgacggcagcatggagggtaaggaaggcagcaccaaagttgaggagaacagcatgaaggcagacaagggtcgcacagaggtcaaccagtgcagcattgacttgggtgaagatgacatggagtttggtgaagacgacatcaatttcagtgaggatgacgtcgaggcagtgaacatcccggagagcctcccacccagtcgtcgtaacagcaacagcaaccctcctctgcccaggtgctaccagtgcaaagctgctaaagtgatcttcatcatcattttctcctatgtgctatccctggggccctactgctttttagcagtcctggccgtgtgggtggatgtcgaaacccaggtaccccagtgggtgatcaccataatcatctggcttttcttcctgcagtgctgcatccacccctatgtctatggctacatgcacaagaccattaagaaggaaatccaggacatgctgaagaagttcttctgcaaggaaaagcccccgaaagaagatagccacccagacctgcccggaacagagggtgggactgaaggcaagattgtcccttcctacgattctgctacttttccttga

SEQ ID NO: 27 - DNA, 28 bp, Artificial Sequence
  Description of Artificial Sequencean artificially synthesized primer sequence
atggaggatctctttagcccctcaattc

SEQ ID NO: 28 - DNA, 28 bp, Artificial Sequence
  Description of Artificial Sequencean artificially synthesized primer sequence
ctagaaggcactttcgcaggagcaaggc

SEQ ID NO: 29 - DNA, 29 bp, Artificial Sequence
  Description of Artificial Sequencean artificially synthesized primer sequence
atggagtcctcacccatcccccagtcatc

SEQ ID NO: 30 - DNA, 29 bp, Artificial Sequence
  Description of Artificial Sequencean artificially synthesized primer sequence
tcatgactccagccggggtgaggcggcag

SEQ ID NO: 31 - DNA, 26 bp, Artificial Sequence
  Description of Artificial Sequencean artificially synthesized primer sequence
atgaaccagactttgaatagcagtgg

SEQ ID NO: 32 - DNA, 28 bp, Artificial Sequence
  Description of Artificial Sequencean artificially synthesized primer sequence
tcaagcccccatctcattggtgcccacg

SEQ ID NO: 33 - DNA, 28 bp, Artificial Sequence
  Description of Artificial Sequencean artificially synthesized primer sequence
atggagaaggtggacatgaatacatcac

SEQ ID NO: 34 - DNA, 29 bp, Artificial Sequence
  Description of Artificial Sequencean artificially synthesized primer sequence
ttacccagatctgttcaaccctgggcatc

SEQ ID NO: 35 - DNA, 28 bp, Artificial Sequence
  Description of Artificial Sequencean artificially synthesized primer sequence
atgacgtccacctgcaccaacagcacgc

SEQ ID NO: 36 - DNA, 29 bp, Artificial Sequence
  Description of Artificial Sequencean artificially synthesized primer sequence
tcaaggaaaagtagcagaatcgtaggaag

SEQ ID NO: 37 - DNA, 20 bp, Artificial Sequence
  Description of Artificial Sequencean artificially synthesized primer sequence
ccaggagcgtttctatgcct

SEQ ID NO: 38 - DNA, 20 bp, Artificial Sequence
  Description of Artificial Sequencean artificially synthesized primer sequence
tgtgatctttgctccctgca

SEQ ID NO: 39 - DNA, 28 bp, Artificial Sequence
  Description of Artificial Sequencean artificially synthesized TaqMan probe sequence
tcagaacctgccagcattgaatagtgcc

SEQ ID NO: 40 - DNA, 20 bp, Artificial Sequence
  Description of Artificial Sequencean artificially synthesized primer sequence
atctgctttgccccgtatgt

SEQ ID NO: 41 - DNA, 20 bp, Artificial Sequence
  Description of Artificial Sequencean artificially synthesized primer sequence
accgccttgctgtaggtcag

SEQ ID NO: 42 - DNA, 22 bp, Artificial Sequence
  Description of Artificial Sequencean artificially synthesized TaqMan probe sequence
tcgtgcccttcgtcaccgtgaa

SEQ ID NO: 43 - DNA, 21 bp, Artificial Sequence
  Description of Artificial Sequencean artificially synthesized primer sequence
cccagcatccataccagaaaa

SEQ ID NO: 44 - DNA, 21 bp, Artificial Sequence
  Description of Artificial Sequencean artificially synthesized primer sequence
ctgtgtccctctcatgccaaa

SEQ ID NO: 45 - DNA, 28 bp, Artificial Sequence
  Description of Artificial Sequencean artificially synthesized TaqMan probe sequence
tgagaaggcagagattcccatccttcct

SEQ ID NO: 46 - DNA, 19 bp, Artificial Sequence
  Description of Artificial Sequencean artificially synthesized primer sequence
tcgccatgagcaacagcat

SEQ ID NO: 47 - DNA, 21 bp, Artificial Sequence
  Description of Artificial Sequencean artificially synthesized primer sequence
cactggacttaccgccattgt

SEQ ID NO: 48 - DNA, 29 bp, Artificial Sequence
  Description of Artificial Sequencean artificially synthesized TaqMan probe sequence
agatcatgttgctccactggaaggcttct

SEQ ID NO: 49 - DNA, 23 bp, Artificial Sequence
  Description of Artificial Sequencean artificially synthesized primer sequence
ggatctctttagcccctcaattc

SEQ ID NO: 50 - DNA, 21 bp, Artificial Sequence
  Description of Artificial Sequencean artificially synthesized primer sequence
aaggtcaggttgagaccccag

SEQ ID NO: 51 - DNA, 25 bp, Artificial Sequence
  Description of Artificial Sequencean artificially synthesized TaqMan probe sequence
aacatttccgtgcccatcttgctgg

SEQ ID NO: 52 - DNA, 21 bp, Artificial Sequence
  Description of Artificial Sequencean artificially synthesized primer sequence
gctgttgactttcgaatccca

SEQ ID NO: 53 - DNA, 23 bp, Artificial Sequence
  Description of Artificial Sequencean artificially synthesized primer sequence
acggaggtagctgtctgacatga

SEQ ID NO: 54 - DNA, 26 bp, Artificial Sequence
  Description of Artificial Sequencean artificially synthesized TaqMan probe sequence
tgagttcctggagcagcaactcacca

SEQ ID NO: 55 - DNA, 20 bp, Artificial Sequence
  Description of Artificial Sequencean artificially synthesized primer sequence
ggctttcgaatgcacaggaa

SEQ ID NO: 56 - DNA, 20 bp, Artificial Sequence
  Description of Artificial Sequencean artificially synthesized primer sequence
ggaagccatgctgaagagga

SEQ ID NO: 57 - DNA, 28 bp, Artificial Sequence
  Description of Artificial Sequencean artificially synthesized TaqMan probe sequence
ttctgcatctatatcctcaacctggcgg

SEQ ID NO: 58 - DNA, 21 bp, Artificial Sequence
  Description of Artificial Sequencean artificially synthesized primer sequence
tggcctcttcaccctctgttt

SEQ ID NO: 59 - DNA, 21 bp, Artificial Sequence
  Description of Artificial Sequencean artificially synthesized primer sequence
atcaagagctggcagtcctga

SEQ ID NO: 60 - DNA, 30 bp, Artificial Sequence
  Description of Artificial Sequencean artificially synthesized TaqMan probe sequence
tccatatcactcgctccttctacctcacca

SEQ ID NO: 61 - DNA, 19 bp, Artificial Sequence
  Description of Artificial Sequencean artificially synthesized primer sequence
ccaaaatgcccatcagcct

SEQ ID NO: 62 - DNA, 20 bp, Artificial Sequence
  Description of Artificial Sequencean artificially synthesized primer sequence
gcactatgttgccgacgaaa

SEQ ID NO: 63 - DNA, 26 bp, Artificial Sequence
  Description of Artificial Sequencean artificially synthesized TaqMan probe sequence
catccgctcaaccgtgctggttatct